(19) United States
(12) Patent Application Publication
(10) Pub. No.: US 2007/0226839 A1
(43) Pub. Date: Sep. 27, 2007

(54) Biotic and abiotic stress tolerance in plants

(75) Inventors: Neal I. Gutterson, Oakland, CA (US); Oliver Ratcliffe, Oakland, CA (US); Emily L. Queen, San Bruno, CA (US); T. Lynne Reuber, San Mateo, CA (US); Karen S. Century, Albany, CA (US); Roger Canales, San Francisco, CA (US)

Correspondence Address: MENDEL 2 C/O MOFO SF, 425 MARKET STREET, SAN FRANCISCO, CA, 94066, US

(73) Assignee: Mendel Biotechnology, Hayward, CA (US)

(21) Appl. No.: 11/699,973

(22) Filed: Jan. 29, 2007

Related U.S. Application Data
(63) Continuation-in-part of application No. PCT/US05/27151, filed on Jul. 29, 2005.
Application No. PCT/US05/27151 is a continuation-in-part of application No. 10/903,236, filed on Jul. 30, 2004.
(63) Continuation-in-part of application No. PCT/US05/46492, filed on Dec. 20, 2005.
(63) Continuation-in-part of application No. 10/546,266, filed on Aug. 19, 2005.
Application No. 10/546,266 is a continuation-in-part of application No. 10/374,780, filed on Feb. 25, 2003.
(63) Continuation-in-part of application No. 10/559,441, filed on Dec. 2, 2005.
Application No. 10/559,441 is a continuation-in-part of application No. 10/456,882, filed on Jun. 6, 2003, now abandoned.
(63) Continuation-in-part of application No. 11/435,388, filed on May 15, 2006.
Application No. 11/435,388 is a continuation-in-part of application No. PCT/US04/37584, filed on Nov. 12, 2004.
Application No. PCT/US04/37584 is a continuation-in-part of application No. 10/714,887, filed on Nov. 13, 2003.
(63) Continuation-in-part of application No. 11/479,226, filed on Jun. 30, 2006.
Application No. 11/479,226 is a continuation-in-part of application No. 09/713,994, filed on Nov. 16, 2000, now abandoned.
(63) Continuation-in-part of application No. 10/374,780, filed on Feb. 25, 2003.
Application No. 10/374,780 is a continuation-in-part of application No. 09/713,994, filed on Nov. 16, 2000, now abandoned.
Application No. 10/374,780 is a continuation-in-part of application No. 09/934,455, filed on Aug. 22, 2001, now abandoned.
Application No. 09/934,455 is a continuation-in-part of application No. 09/713,994, filed on Nov. 16, 2000, now abandoned.
Application No. 09/934,455 is a continuation-in-part of application No. 09/837,944, filed on Apr. 18, 2001, now abandoned.
(63) Continuation-in-part of application No. 10/225,068, filed on Aug. 9, 2002, now Pat. No. 7,193,129.
Application No. 10/225,068 is a continuation-in-part of application No. 09/837,944, filed on Apr. 18, 2001, now abandoned.
Application No. 10/225,068 is a continuation-in-part of application No. 10/171,468, filed on Jun. 14, 2002, now abandoned.
(63) Continuation-in-part of application No. 10/225,066, filed on Aug. 9, 2002, now Pat. No. 7,238,860.
Application No. 10/225,066 is a continuation-in-part of application No. 09/837,944, filed on Apr. 18, 2001, now abandoned.
Application No. 10/225,066 is a continuation-in-part of application No. 10/171,468, filed on Jun. 14, 2002, now abandoned.
(63) Continuation-in-part of application No. 11/642,814, filed on Dec. 20, 2006.
Application No. 11/642,814 is a division of application No. 10/666,642, filed on Sep. 18, 2003, now Pat. No. 7,196,245.
(63) Continuation-in-part of application No. 10/714,887, filed on Nov. 13, 2003.
(63) Continuation-in-part of application No. 10/903,236, filed on Jul. 30, 2004.
(60) Provisional application No. 60/638,353, filed on Dec. 20, 2004.
(60) Provisional application No. 60/166,228, filed on Nov. 17, 1999.
(60) Provisional application No. 60/166,228, filed on Nov. 17, 1999.
(60) Provisional application No. 60/310,847, filed on Aug. 9, 2001.
(60) Provisional application No. 60/336,049, filed on Nov. 19, 2001.
(60) Provisional application No. 60/338,692, filed on Dec. 11, 2001.
(60) Provisional application No. 60/310,847, filed on Aug. 9, 2001.
(60) Provisional application No. 60/336,049, filed on Nov. 19, 2001.
(60) Provisional application No. 60/338,692, filed on Dec. 11, 2001.
(60) Provisional application No. 60/411,837, filed on Sep. 18, 2002.
(60) Provisional application No. 60/465,809, filed on Apr. 24, 2003.

(51) Int. Cl. A01H 1/00; C12N 15/82

(52) U.S. Cl. 800/279; 800/289

(57) ABSTRACT

The invention relates to plant transcription factor polypeptides, polynucleotides that encode them, homologs from a variety of plant species, and methods of using the polynucleotides and polypeptides to produce transgenic plants having advantageous properties, including resistance to disease and tolerance to low nitrogen, drought, and other abiotic stresses, as compared to wild-type or control plants.

### CROSS-REFERENCE TO RELATED APPLICATIONS

This application is a Continuation-In-Part of International Application No. PCT/US2005/027151, filed Jul. 29, 2005, which is a Continuation-In-Part of U.S. application Ser. No. 10/903,236, filed Jul. 30, 2004. This application is also a Continuation-In-Part of International Application Number PCT/US2005/046492, filed on Dec. 20, 2005, which claims the benefit of U.S. Provisional Application Ser. No. 60/638,353, filed Dec. 20, 2004. This application is also a Continuation-In-Part of U.S. application Ser. No. 10/546,266, filed Aug. 19, 2005, which is a US National Stage Filing of International Application Number PCT/US2004/005654, filed Feb. 25, 2004, which is a Continuation-In-Part of U.S. application Ser. No. 10/374,780, filed Feb. 25, 2003. This application is also a Continuation-In-Part of U.S. application Ser. No. 10/559,441, filed Dec. 2, 2005, which is a US National Stage Filing of International Application No. PCT/US2004/17768, filed Jun. 4, 2004, which is a Continuation-In-Part of U.S. application Ser. No. 10/456,882, filed Jun. 6, 2003 (abandoned). This application is also a Continuation-In-Part of U.S. application Ser. No. 11/435,388, filed May 15, 2006, which is a Continuation-In-Part of International Application No. PCT/US2004/37584, filed Nov. 12, 2004, which is a Continuation-In-Part of U.S. application Ser. No. 10/714,887, filed Nov. 13, 2003. This application is also a Continuation-In-Part of U.S. application Ser. No. 11/479,226, filed Jun. 30, 2006, which is a Continuation-In-Part of U.S. application Ser. No. 09/713,994, filed Nov. 16, 2000 (abandoned), which claims the benefit of U.S. Provisional Application Ser. No. 60/166,228, filed Nov. 17, 1999. This application is also a Continuation-In-Part of U.S. application Ser. No. 10/374,780, filed Feb. 25, 2003, which is a Continuation-In-Part of U.S. application Ser. No. 09/713,994, filed Nov. 16, 2000 (abandoned), which claims the benefit of U.S. Provisional Application Ser. No. 60/166,228, filed Nov. 17, 1999; U.S. application Ser. No. 10/374,780 is also a Continuation-In-Part of U.S. application Ser. No. 09/934,455, filed Aug. 22, 2001 (abandoned), which is a Continuation-In-Part of U.S. application Ser. No. 09/713,994, filed Nov. 16, 2000 (abandoned), which is a Continuation-In-Part of U.S. application Ser. No. 09/837,944, filed Apr. 18, 2001 (abandoned). This application is also a Continuation-In-Part of U.S. application Ser. No. 10/255,068, filed Aug. 9, 2002, which is a Continuation-In-Part of U.S. application Ser. No. 09/837,944, filed Apr. 18, 2001 (abandoned); application Ser. No. 10/255,068 is also a Continuation-In-Part of U.S. application Ser. No. 10/171,468, filed Jun. 14, 2002 (abandoned); application Ser. No. 10/255,068 also claims the benefit of U.S. Provisional Application Ser. No. 60/310,847, filed Aug. 9, 2001; application Ser. No. 10/255,068 also claims the benefit of U.S. Provisional Application Ser. No. 60/336,049, filed Nov. 19, 2001; and application Ser. No. 10/255,068 also claims the benefit of U.S. Provisional Application Ser. No. 60/338,692, filed Dec. 11, 2001. This application is also a Continuation-In-Part of U.S. application Ser. No. 10/225,066, filed Aug. 9, 2002, which is a Continuation-In-Part of U.S. application Ser. No. 09/837,944, filed Apr. 18, 2001 (abandoned); application Ser. No. 10/225,066 is also a Continuation-In-Part of U.S. application Ser. No. 10/171,468, filed Jun. 14, 2002 (abandoned); application Ser. No. 10/225,066 also claims the benefit of U.S. Provisional Application Ser. No. 60/310,847, filed Aug. 9, 2001; application Ser. No. 10/225,066 also claims the benefit of U.S. Provisional Application Ser. No. 60/336,049, filed Nov. 19, 2001; and application Ser. No. 10/225,066 also claims the benefit of U.S. Provisional Application Ser. No. 60/338,692, filed Dec. 11, 2001. This application is also a Continuation-In-Part of U.S. application Ser. No. 11/642,814, filed Dec. 20, 2006, which is a Divisional of U.S. application Ser. No. 10/666,642, filed Sep. 18, 2003, which claims the benefit of U.S. Provisional Application Ser. No. 60/411,837, filed Sep. 18, 2002; and application Ser. No. 10/666,642 also claims the benefit of U.S. Provisional Application Ser. No. 60/465,809, filed Apr. 24, 2003. This application is also a Continuation-In-Part of U.S. application Ser. No. 10/714,887, filed Nov. 13, 2003. This application is also a Continuation-In-Part of U.S. application Ser. No. 10/903,236, filed Jul. 30, 2004. All of the preceding applications are hereby incorporated by reference in their entirety

### FIELD OF THE INVENTION

The present invention relates to increasing a plant's resistance to disease and tolerance to abiotic stress, and the yield that may be obtained from a plant.

### BACKGROUND OF THE INVENTION

Studies from a diversity of prokaryotic and eukaryotic organisms suggest a gradual evolution of biochemical and physiological mechanisms and metabolic pathways. Despite different evolutionary pressures, proteins that regulate the cell cycle in yeast, plant, nematode, fly, rat, and man have common chemical or structural features and modulate the same general cellular activity. A comparison of gene sequences with known structure and/or function from one plant species, for example, Arabidopsis thaliana, with those from other plants, allows researchers to develop models for manipulating a plant's traits and developing varieties with valuable properties.

One important way to control cellular processes is through transcription factors—proteins that influence the expression of a particular gene or sets of genes. Because transcription factors are key controlling elements of biological pathways, altering the expression levels of one or more transcription factors can change entire biological pathways in an organism. Manipulating a plant's biochemical, developmental, or phenotypic characteristics by altering a transcription factor expression can result in plants and crops with new and/or improved commercially valuable properties, including improved survival and yield during periods of abiotic stress, including hyperosmotic stresses such as drought, high salt, other abiotic stresses such as cold or heat, or when the plants contend with low nitrogen conditions.

We have identified polynucleotides encoding transcription factors, including Arabidopsis sequences G1792, G1791, G1795, G30, soy sequences G3518, G3519 and G3520, rice sequences G3380, G3381, G3383, G3515, and G3737, corn sequences G3516, G3517 and G3739, and equivalogs listed in the Sequence Listing from a variety of other species, developed transgenic plants using some of these polynucleotides from diverse species, and analyzed the plants for their resistance to disease and their tolerance to abiotic stress and low nitrogen conditions. In so doing, we have identified important polynucleotide and polypeptide sequences for producing commercially valuable plants and crops as well as the methods for making them and using them. Other aspects and embodiments of the invention are described below and can be derived from the teachings of this disclosure as a whole.

### SUMMARY OF THE INVENTION

The present invention describes polynucleotides that may be introduced into plants. The polynucleotides encode transcription factor polypeptides that have the useful properties of increasing increased abiotic or biotic stress tolerance, increased tolerance to low nitrogen, and/or altered sensing of carbon-nitrogen (C/N) balance. The present invention thus may be used to increase a plant's tolerance to resistance to biotic stress, or tolerance to abiotic stress, including multiple abiotic stresses, which may further include hyperosmotic stresses such as high salt or drought. This method is accomplished by first providing an expression vector and then introducing the expression vector into a plant to produce a transformed plant. The expression vector contains both a regulatory element and a polynucleotide sequence. The regulatory element controls the expression of the polynucleotide sequence. The polynucleotide encodes a member of the G1792 clade of transcription factor polypeptides, which are shown in the present invention to comprise two distinct conserved domains: an AP2 domain and an EDLL domain, in order from N-terminal to C-terminal. The EDLL domain is characterized by, in order from N-terminal to C-terminal, a glutamic acid residue, an aspartic acid residue, and two leucine residues. The consensus sequence for the EDLL domain is represented by SEQ ID NO: 63. After a target plant is transformed with the expression vector, which confers increased disease resistance or abiotic stress tolerance by virtue of the overexpression of the G1792 clade member, the transformed plant is grown.

The invention also pertains to a method for producing a plant with greater disease resistance or abiotic stress tolerance than a control plant. This method is performed by providing the expression vector just described. After transforming a target plant with this expression vector, a transformed plant with greater disease resistance or abiotic stress tolerance than a control plant is the result. Disease pathogens may include fungal pathogens. Abiotic stresses to which the plant may be more tolerant include low nitrogen conditions, hyperosmotic stresses such as high salt and drought, and other abiotic stresses such as heat and cold.

The invention also encompasses transgenic plants that have greater tolerance to multiple abiotic stress tolerances than a control plant, wherein the transgenic plants are produced by the above methods.

The invention is further directed to seed produced from any of the transformed plants produced by the methods disclosed or claimed herein.

The methods encompassed by the invention may be extended to propagation techniques used to generate plants. For example, a target plant that has been transformed with a polynucleotide encoding a G1792 polypeptide clade member and that has greater abiotic stress tolerance than a wild-type or non-transformed control may be “selfed” (i.e., self-pollinated) or crossed with another plant to produce seed. Progeny plants may be grown from this seed, thus generating transformed progeny plants with increased resistance to disease or tolerance to abiotic stress, as compared to wild-type, control or non-transformed plants of the same species.

### BRIEF DESCRIPTION OF THE SEQUENCE LISTING AND DRAWINGS

The Sequence Listing provides exemplary polynucleotide and polypeptide sequences of the invention. The traits associated with the use of the sequences are included in the Examples.

FIG. 1 shows a conservative estimate of phylogenetic relationships among the orders of flowering plants (modified from Angiosperm Phylogeny Group (1998) Ann. Missouri Bot Gard. 84: 1-49). Those plants with a single cotyledon (monocots) are a monophyletic clade nested within at least two major lineages of dicots; the eudicots are further divided into rosids and asterids. Arabidopsis is a rosid eudicot classified within the order Brassicales; rice is a member of the monocot order Poales. FIG. 1 was adapted from Daly et al. (2001). Plant Physiol. 127: 1328-1333.

FIG. 2 shows a phylogenic dendogram depicting phylogenetic relationships of higher plant taxa, including clades containing tomato and Arabidopsis; adapted from Ku et al. (2000) Proc. Natl. Acad. Sci. USA 97: 9121-9126; and Chase et al. (1993) Ann. Missouri Bot. Gard. 80: 528-580.

FIGS. 3A-3L represent a multiple amino acid sequence alignment of G1792 orthologs and paralogs. Clade orthologs and paralogs are indicated by the black bar on the left side of the figure. Conserved regions of identity are boxed and appear in boldface, while conserved sequences of similarity are boxed and appear as plain text. The AP2 conserved domains span alignment coordinates 196-254. The S conserved domain spans alignment coordinates of 301-304. The EDLL conserved domain (SEQ ID NO: 63) spans the alignment coordinates of 391-406 (FIGS. 3J-3K; see also FIG. 4). Abbreviations in this figure include: At Arabidopsis thaliana; Os Oryza sativa; Zm Zea mays; Ta Triticum aestivum; Gm Glycine max; Mt Medicago truncatula.

FIG. 4 shows a novel conserved domain for the G1792 clade, herein referred to as the “EDLL domain” (SEQ ID NO: 63). All clade members contain a glutamic acid residue at position 3, an aspartic acid residue at position 8, and leucine residues at positions 12 and 16 of the domain.

FIG. 5 illustrates the relationship of G1792 and related sequences in this phylogenetic tree of the G1792 clade. The tree building method used was “Neighbor Joining” with “Systematic Tie-Breaking” and Bootstrapping with 1000 replicates. The AP2 domains (as listed in Table 1) were used to build the phylogeny. The members of the G1792 clade are shown within the large box.

### DETAILED DESCRIPTION

The present invention relates to polynucleotides and polypeptides for modifying phenotypes of plants, particularly those associated with increased tolerance to low nitrogen and abiotic stress. Throughout this disclosure, various information sources are referred to and/or are specifically incorporated. The information sources include scientific journal articles, patent documents, textbooks, and World Wide Web browser-inactive page addresses, for example. While the reference to these information sources clearly indicates that they can be used by one of skill in the art, each and every one of the information sources cited herein are specifically incorporated in their entirety, whether or not a specific mention of “incorporation by reference” is noted. The contents and teachings of each and every one of the information sources can be relied on and used to make and use embodiments of the invention.

As used herein and in the appended claims, the singular forms “a”, “an”, and “the” include the plural reference unless the context clearly dictates otherwise. Thus, for example, a reference to “a host cell” includes a plurality of such host cells, and a reference to “a stress” is a reference to one or more stresses and equivalents thereof known to those skilled in the art, and so forth.

Definitions

“Nucleic acid molecule” refers to an oligonucleotide, polynucleotide or any fragment thereof. It may be DNA or RNA of genomic or synthetic origin, double-stranded or single-stranded, and combined with carbohydrate, lipids, protein, or other materials to perform a particular activity such as transformation or form a useful composition such as a peptide nucleic acid (PNA).

“Polynucleotide” is a nucleic acid molecule comprising a plurality of polymerized nucleotides, e.g., at least about 15 consecutive polymerized nucleotides, optionally at least about 30 consecutive nucleotides, at least about 50 consecutive nucleotides. A polynucleotide may be a nucleic acid, oligonucleotide, nucleotide, or any fragment thereof. In many instances, a polynucleotide comprises a nucleotide sequence encoding a polypeptide (or protein) or a domain or fragment thereof. Additionally, the polynucleotide may comprise a promoter, an intron, an enhancer region, a polyadenylation site, a translation initiation site, 5′ or 3′ untranslated regions, a reporter gene, a selectable marker, or the like. The polynucleotide can be single stranded or double stranded DNA or RNA. The polynucleotide optionally comprises modified bases or a modified backbone. The polynucleotide can be, e.g., genomic DNA or RNA, a transcript (such as an mRNA), a cDNA, a PCR product, a cloned DNA, a synthetic DNA or RNA, or the like. The polynucleotide can be combined with carbohydrate, lipids, protein, or other materials to perform a particular activity such as transformation or form a useful composition, such as a peptide nucleic acid (PNA). The polynucleotide can comprise a sequence in either sense or antisense orientations. “Oligonucleotide” is substantially equivalent to the terms amplimer, primer, oligomer, element, target, and probe and is preferably single stranded.

“Gene” or “gene sequence” refers to the partial or complete coding sequence of a gene, its complement, and its 5′ or 3′ untranslated regions. A gene is also a functional unit of inheritance, and in physical terms is a particular segment or sequence of nucleotides along a molecule of DNA (or RNA, in the case of RNA viruses) involved in producing a polypeptide chain. The latter may be subjected to subsequent processing such as splicing and folding to obtain a functional protein or polypeptide. A gene may be isolated, partially isolated, or be found with an organism's genome. By way of example, a transcription factor gene encodes a transcription factor polypeptide, which may be functional or require processing to function as an initiator of transcription.

Operationally, genes may be defined by the cis-trans test, a genetic test that determines whether two mutations occur in the same gene and which may be used to determine the limits of the genetically active unit (Rieger et al. (1976) Glossary of Genetics and Cytogenetics: Classical and Molecular, 4th ed., Springer Verlag, Berlin). A gene generally includes regions preceding (“leaders”; upstream) and following (“trailers”; downstream) the coding region. A gene may also include intervening, non-coding sequences, referred to as “introns”, located between individual coding segments, referred to as “exons”. Most genes have an associated promoter region, a regulatory sequence 5′ of the transcription initiation codon (there are some genes that do not have an identifiable promoter). The function of a gene may also be regulated by enhancers, operators, and other regulatory elements.

A “recombinant polynucleotide” is a polynucleotide that is not in its native state, e.g., the polynucleotide comprises a nucleotide sequence not found in nature, or the polynucleotide is in a context other than that in which it is naturally found, e.g., separated from nucleotide sequences with which it typically is in proximity in nature, or adjacent (or contiguous with) nucleotide sequences with which it typically is not in proximity. For example, the sequence at issue can be cloned into a vector, or otherwise recombined with one or more additional nucleic acid.

An “isolated polynucleotide” is a polynucleotide whether naturally occurring or recombinant, that is present outside the cell in which it is typically found in nature, whether purified or not. Optionally, an isolated polynucleotide is subject to one or more enrichment or purification procedures, e.g., cell lysis, extraction, centrifugation, precipitation, or the like.

A “polypeptide” is an amino acid sequence comprising a plurality of consecutive polymerized amino acid residues e.g., at least about 15 consecutive polymerized amino acid residues. In many instances, a polypeptide comprises a polymerized amino acid residue sequence that is a transcription factor or a domain or portion or fragment thereof. Additionally, the polypeptide may comprise 1) a localization domain, 2) an activation domain, 3) a repression domain, 4) an oligomerization domain, or 5) a DNA-binding domain, or the like. The polypeptide optionally comprises modified amino acid residues, naturally occurring amino acid residues not encoded by a codon, non-naturally occurring amino acid residues.

“Protein” refers to an amino acid sequence, oligopeptide, peptide, polypeptide or portions thereof whether naturally occurring or synthetic.

“Portion”, as used herein, refers to any part of a protein used for any purpose, but especially for the screening of a library of molecules which specifically bind to that portion or for the production of antibodies.

A “recombinant polypeptide” is a polypeptide produced by translation of a recombinant polynucleotide. A “synthetic polypeptide” is a polypeptide created by consecutive polymerization of isolated amino acid residues using methods well known in the art. An “isolated polypeptide,” whether a naturally occurring or a recombinant polypeptide, is more enriched in (or out of) a cell than the polypeptide in its natural state in a wild-type cell, e.g., more than about 5% enriched, more than about 10% enriched, or more than about 20%, or more than about 50%, or more, enriched, i.e., alternatively denoted: 105%, 110%, 120%, 150% or more, enriched relative to wild type standardized at 100%. Such an enrichment is not the result of a natural response of a wild-type plant. Alternatively, or additionally, the isolated polypeptide is separated from other cellular components with which it is typically associated, e.g., by any of the various protein purification methods herein.

“Homology” refers to sequence similarity between a reference sequence and at least a fragment of a newly sequenced clone insert or its encoded amino acid sequence.

“Hybridization complex” refers to a complex between two nucleic acid molecules by virtue of the formation of hydrogen bonds between purines and pyrimidines.

“Identity” or “similarity” refers to sequence similarity between two or more polynucleotide sequences, or two or more polypeptide sequences, with identity being a more strict comparison. The phrases “percent identity” and “% identity” refer to the percentage of identical bases or residues at corresponding positions found in a comparison of two or more sequences (when a position in the compared sequence is occupied by the same nucleotide base or amino acid, then the molecules are identical at that position). “Sequence similarity” refers to the percentage of bases that are similar in the corresponding positions of two or more polynucleotide sequences. A degree of homology or similarity of polypeptide sequences is a function of the number of similar amino acid residues at positions shared by the polypeptide sequences. Two or more sequences can be anywhere from 0-1.00% similar, or any integer value therebetween. Identity or similarity can be determined by comparing a position in each sequence that may be aligned for purposes of comparison.

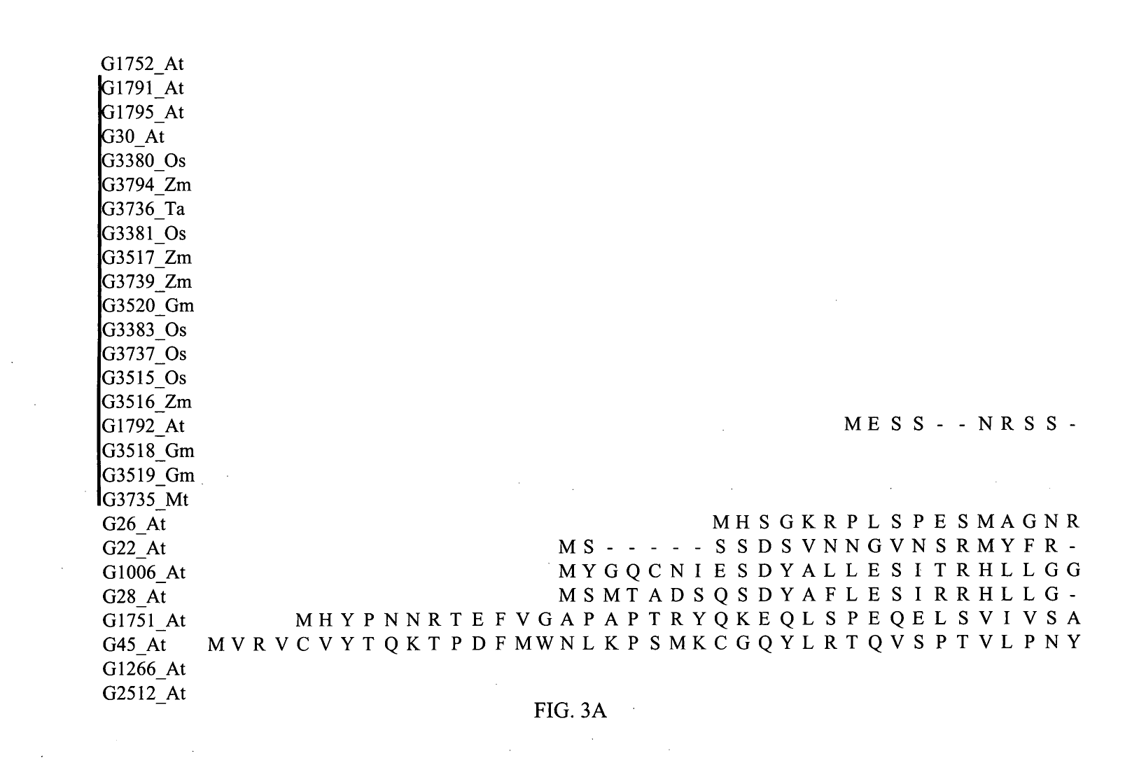

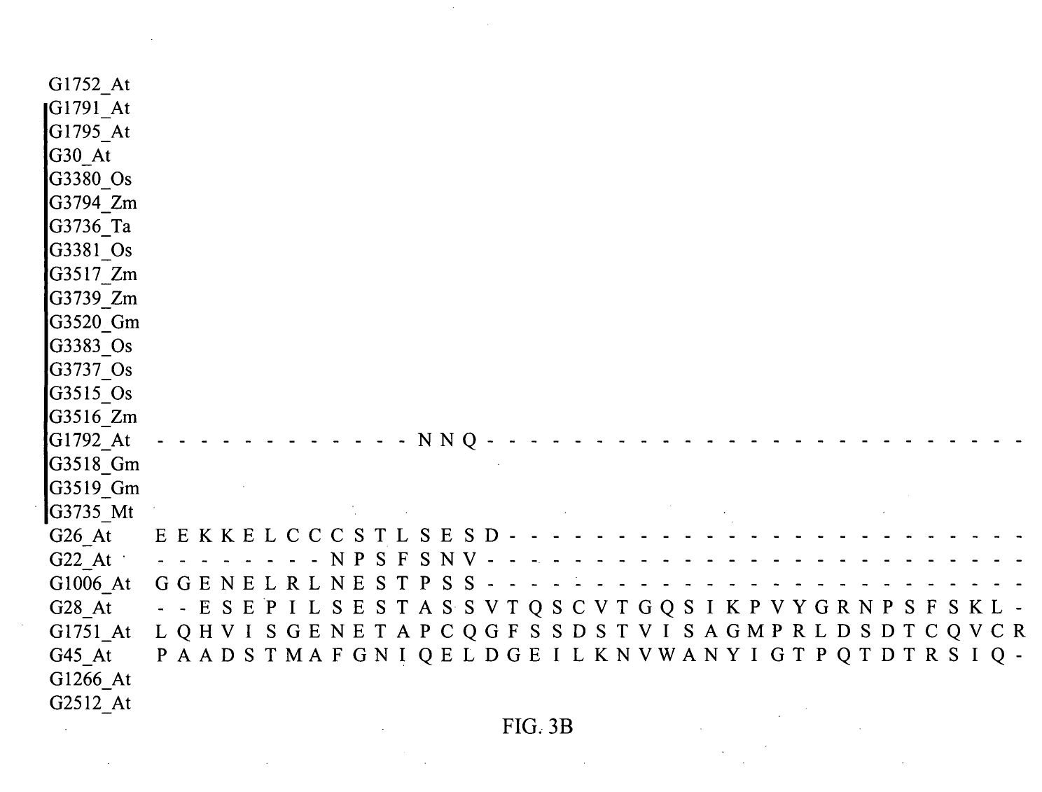

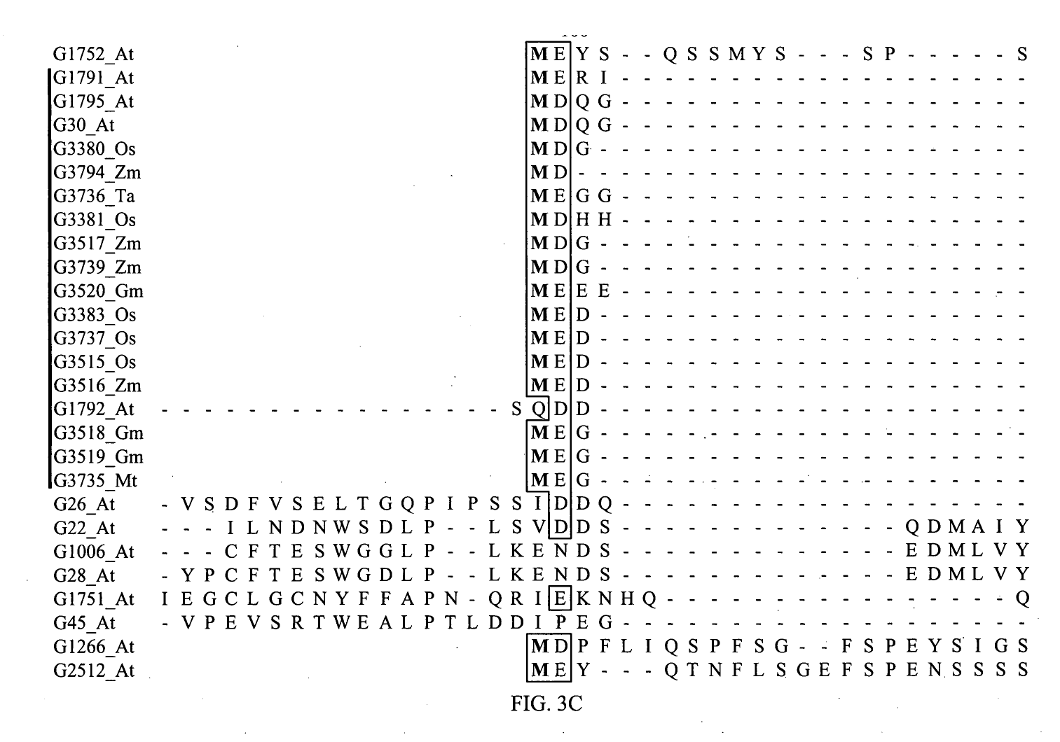

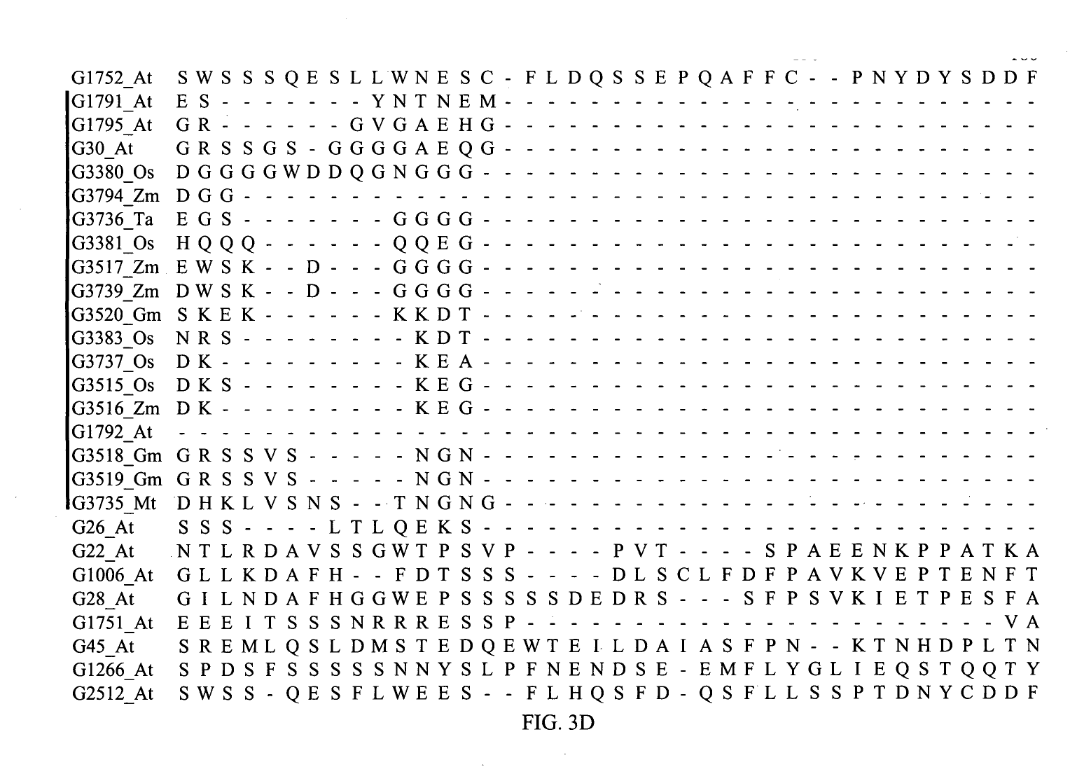

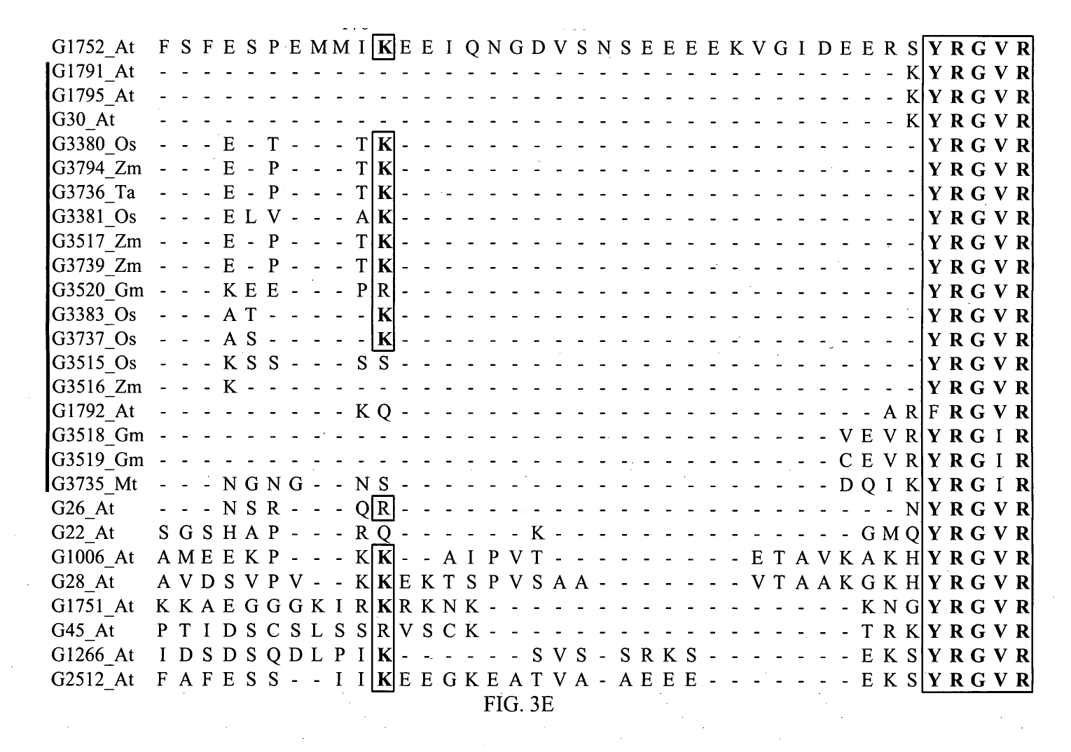

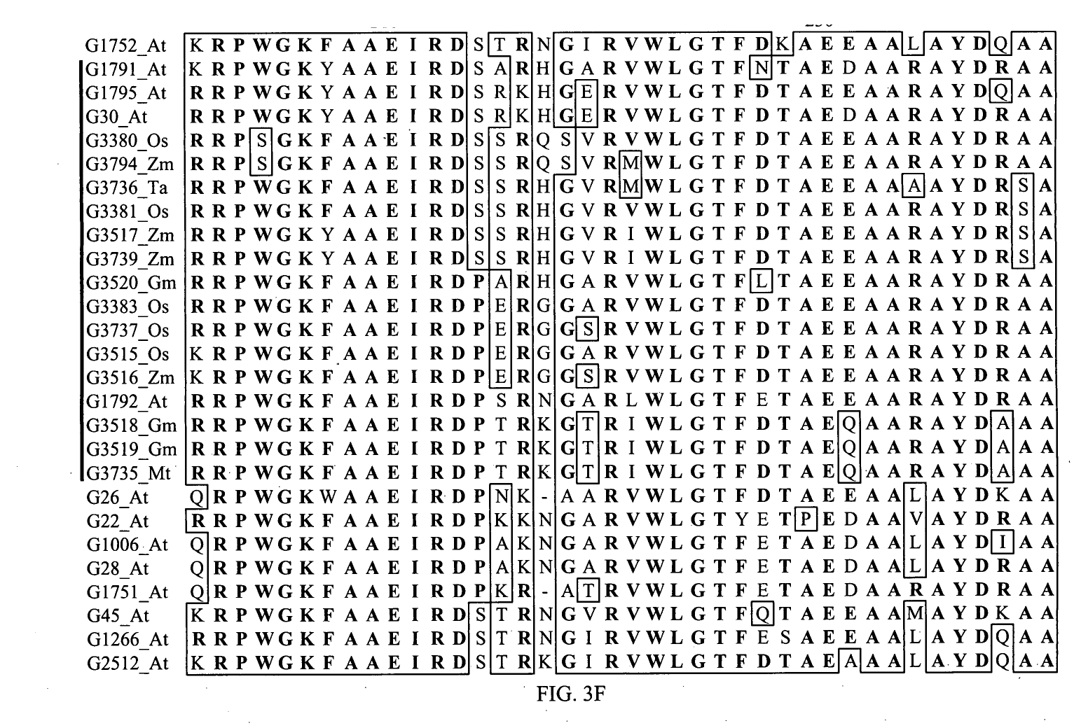

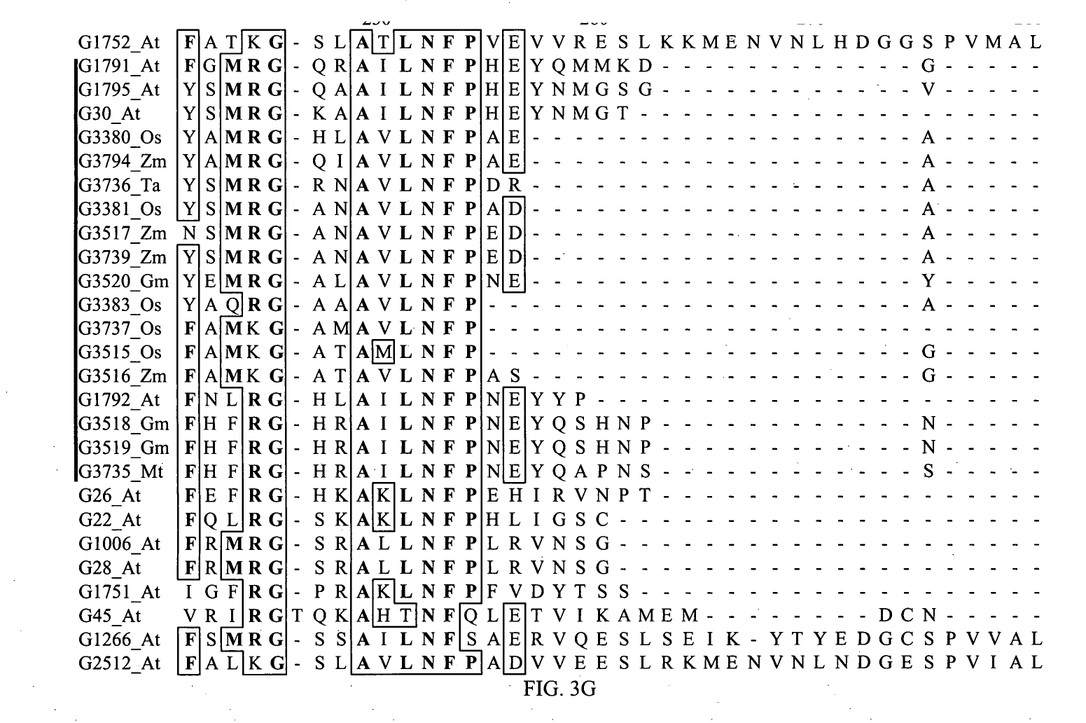

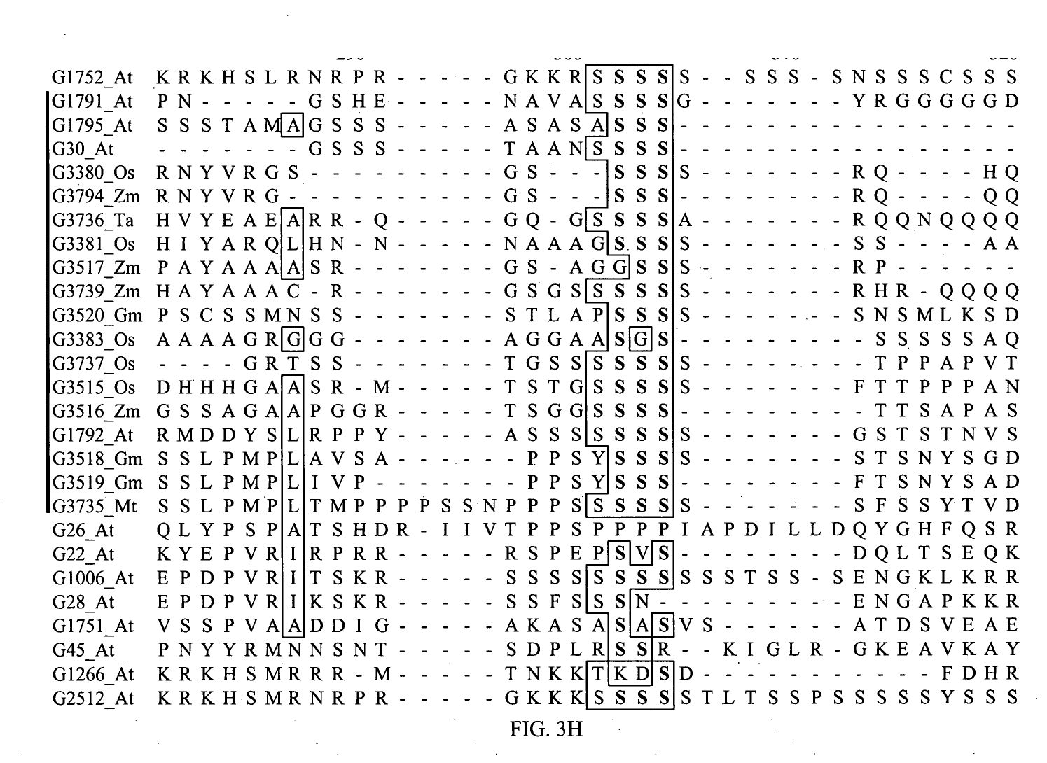

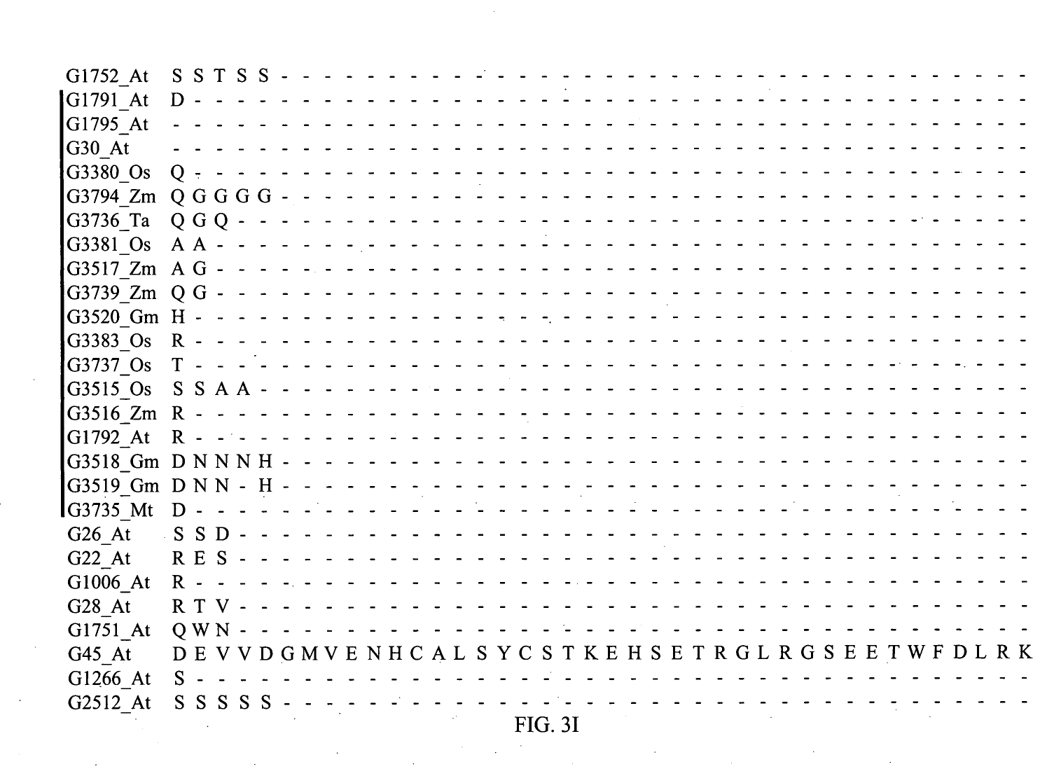

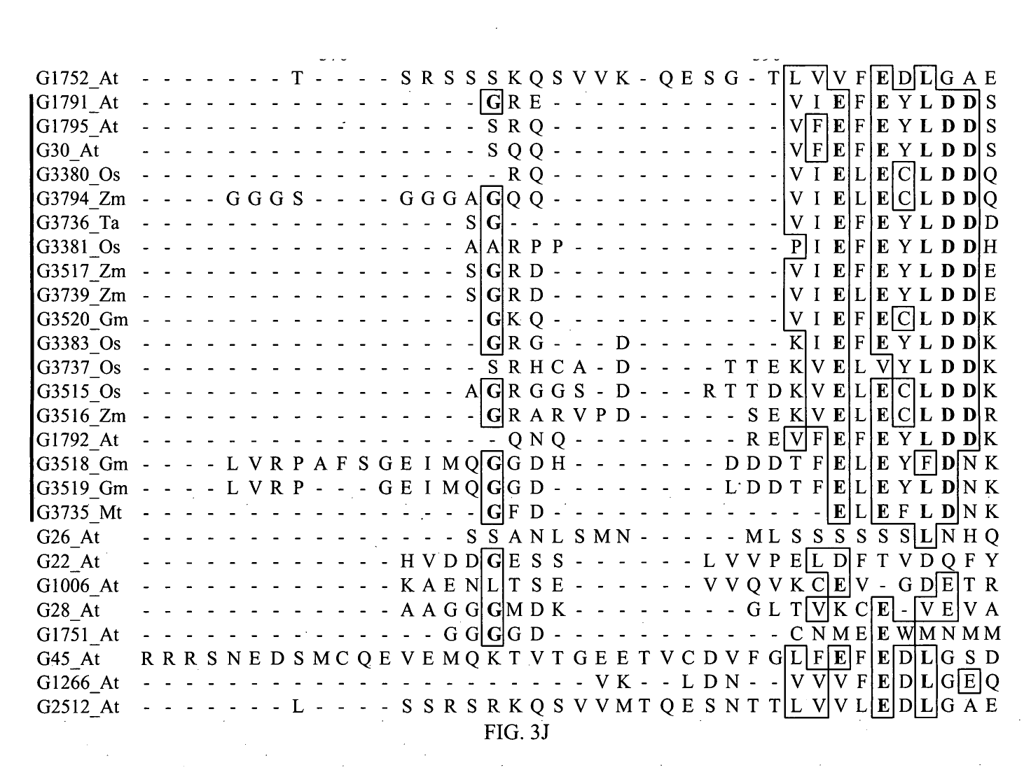

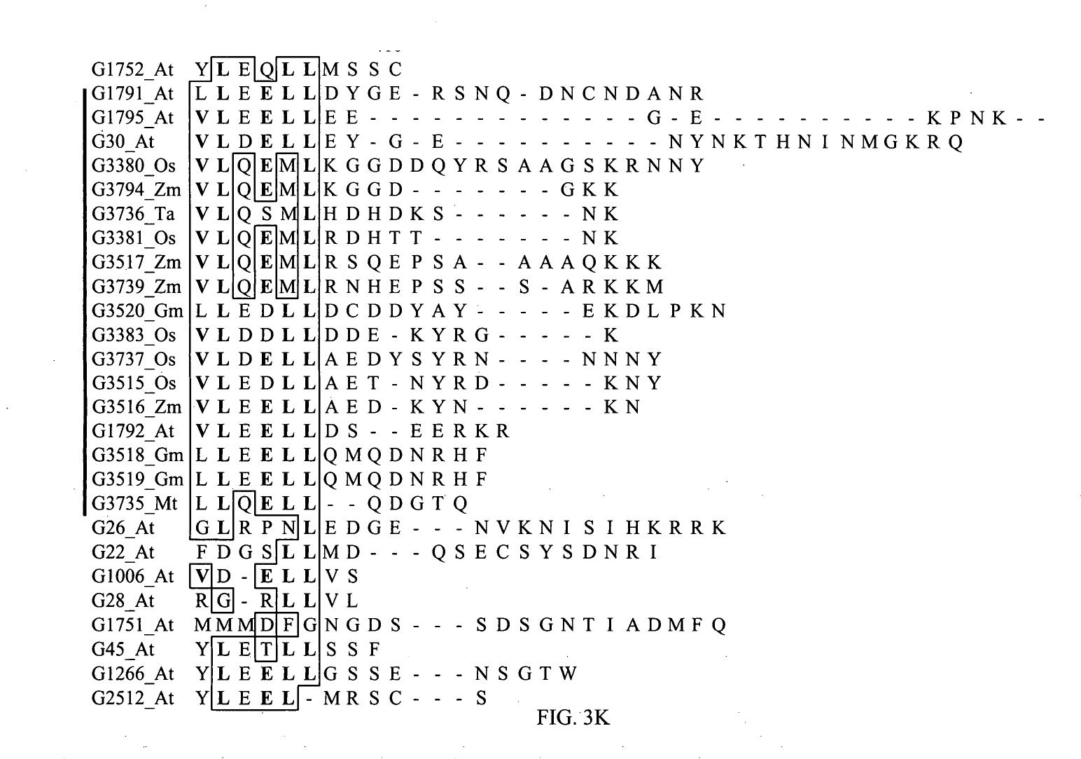

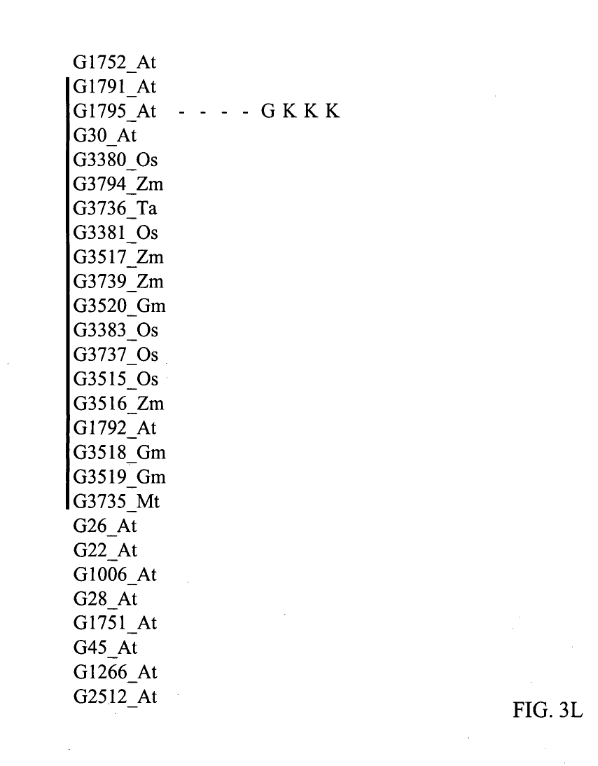

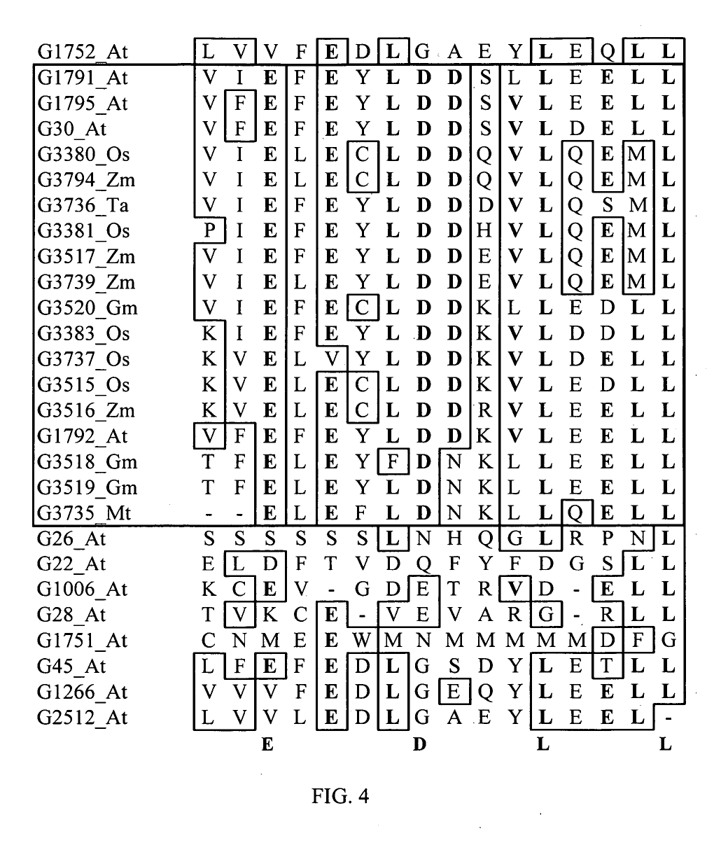

“Alignment” refers to a number of nucleotide bases or amino acid residue sequences aligned by lengthwise comparison so that components in common (i.e., nucleotide bases or amino acid residues) may be visually and readily identified. The fraction or percentage of components in common is related to the homology or identity between the sequences. Alignments such as those of FIGS. 3A-L or FIG. 4 may be used to identify conserved domains and relatedness within these domains. An alignment may suitably be determined by means of computer programs known in the art, such as MACVECTOR software (1999) (Accelrys, Inc., San Diego, Calif.).

A “conserved domain” or “conserved region” as used herein refers to a region in heterologous polynucleotide or polypeptide sequences where there is a relatively high degree of sequence identity between the distinct sequences. An “AP2 domain”, such as is found in a member of AP2 transcription factor family, is an example of a conserved domain. With respect to polynucleotides encoding presently disclosed transcription factors, a conserved domain is preferably at least 10 base pairs (bp) in length. A “conserved domain”, with respect to presently disclosed AP2 polypeptides refers to a domain within a transcription factor family that exhibits a higher degree of sequence homology, such as at least 62% sequence identity including conservative substitutions, and more preferably at least 65% sequence identity, and even more preferably at least 69%, or at least about 70%, or at least about 72%, or at least about 73%, or at least about 74%, or at least about 74%, or at least about 75%, or at least about 76%, or at least about 77%, or at least about 78%, or at least about 79%, or at least about 80%, or at least about 82%, or at least about 83%, or at least about 85%, or at least about 87%, or at least about 90%, or at least about 95%, or at least about 98% amino acid residue sequence identity to the conserved domain. A fragment or domain can be referred to as outside a conserved domain, outside a consensus sequence, or outside a consensus DNA-binding site that is known to exist or that exists for a particular transcription factor class, family, or sub-family. In this case, the fragment or domain will not include the exact amino acids of a consensus sequence or consensus DNA-binding site of a transcription factor class, family or sub-family, or the exact amino acids of a particular transcription factor consensus sequence or consensus DNA-binding site. Furthermore, a particular fragment, region, or domain of a polypeptide, or a polynucleotide encoding a polypeptide, can be “outside a conserved domain” if all the amino acids of the fragment, region, or domain fall outside of a defined conserved domain(s) for a polypeptide or protein. Sequences having lesser degrees of identity but comparable biological activity are considered to be equivalents.

As one of ordinary skill in the art recognizes, conserved domains may be identified as regions or domains of identity to a specific consensus sequence (for example, Riechmann et al. (2000) Science 290: 2105-2110). Thus, by using alignment methods well known in the art, the conserved domains of the plant transcription factors for the AP2 proteins may be determined.

Conserved domains for members of the G1792 clade of transcription factor polypeptides (or simply the “G1792 clade”), including SEQ ID NO: 2, 4, 6, 8, 10, 12, 14, 16, 18, 20, 22, 24, 26, 28, 30, 32, 34 and 36, are listed in Table 1. A comparison of these conserved domains with other sequences would allow one of skill in the art to identify AP2 or EDLL domains in the polypeptides listed or referred to in this disclosure, as well as other polypeptides not presented in this disclosure, but which comprise these domains.

“Complementary” refers to the natural hydrogen bonding by base pairing between purines and pyrimidines. For example, the sequence A-C-G-T (5′->3′) forms hydrogen bonds with its complements A-C-G-T (5′->3′) or A-C-G-U (5′->3′). Two single-stranded molecules may be considered partially complementary, if only some of the nucleotides bond, or “completely complementary” if all of the nucleotides bond. The degree of complementarity between nucleic acid strands affects the efficiency and strength of the hybridization and amplification reactions. “Fully complementary” refers to the case where bonding occurs between every base pair and its complement in a pair of sequences, and the two sequences have the same number of nucleotides.

The terms “highly stringent” or “highly stringent condition” refer to conditions that permit hybridization of DNA strands whose sequences are highly complementary, wherein these same conditions exclude hybridization of significantly mismatched DNAs. Polynucleotide sequences capable of hybridizing under stringent conditions with the polynucleotides of the present invention may be, for example, variants of the disclosed polynucleotide sequences, including allelic or splice variants, or sequences that encode orthologs or paralogs of presently disclosed polypeptides. Nucleic acid hybridization methods are disclosed in detail by Kashima et al. (1985) Nature 313:402-404, and Sambrook et al. (1989) Molecular Cloning: A Laboratory Manual, 2nd Ed., Cold Spring Harbor Laboratory, Cold Spring Harbor, N.Y.; and by Haymes et al. “Nucleic Acid Hybridization: A Practical Approach”, IRL Press, Washington, D.C. (1985), which references are incorporated herein by reference.

In general, stringency is determined by the temperature, ionic strength, and concentration of denaturing agents (e.g., formamide) used in a hybridization and washing procedure (a more detailed description of establishing and determining stringency is disclosed below). The degree to which two nucleic acids hybridize under various conditions of stringency is correlated with the extent of their similarity. Thus, similar nucleic acid sequences from a variety of sources, such as within a plant's genome (as in the case of paralogs) or from another plant (as in the case of orthologs) that may perform similar functions can be isolated on the basis of their ability to hybridize with known transcription factor sequences. Numerous variations are possible in the conditions and means by which nucleic acid hybridization can be performed to isolate transcription factor sequences having similarity to transcription factor sequences known in the art and are not limited to those explicitly disclosed herein. Such an approach may be used to isolate polynucleotide sequences having various degrees of similarity with disclosed transcription factor sequences, such as, for example, encoded transcription factors having 62% or greater identity with the AP2 domain of disclosed transcription factors.

Regarding the terms “paralog” and “ortholog”, homologous polynucleotide sequences and homologous polypeptide sequences may be paralogs or orthologs of the claimed polynucleotide or polypeptide sequence. Orthologs and paralogs are evolutionarily related genes that have similar sequence and similar functions. Orthologs are structurally related genes in different species that are derived by a speciation event. Paralogs are structurally related genes within a single species that are derived by a duplication event. Sequences that are sufficiently similar to one another will be appreciated by those of skill in the art and may be based upon percentage identity of the complete sequences, percentage identity of a conserved domain or sequence within the complete sequence, percentage similarity to the complete sequence, percentage similarity to a conserved domain or sequence within the complete sequence, and/or an arrangement of contiguous nucleotides or peptides particular to a conserved domain or complete sequence. Sequences that are sufficiently similar to one another will also bind in a similar manner to the same DNA binding sites of transcriptional regulatory elements using methods well known to those of skill in the art.

The term “equivalog” describes members of a set of homologous proteins that are conserved with respect to function since their last common ancestor. Related proteins are grouped into equivalog families, and otherwise into protein families with other hierarchically defined homology types. This definition is provided at the Institute for Genomic Research (TIGR) World Wide Web (www) website, “tigr.org” under the heading “Terms associated with TIGRFAMs”.

The term “variant”, as used herein, may refer to polynucleotides or polypeptides that differ from the presently disclosed polynucleotides or polypeptides, respectively, in sequence from each other, and as set forth below.

With regard to polynucleotide variants, differences between presently disclosed polynucleotides and polynucleotide variants are limited so that the nucleotide sequences of the former and the latter are closely similar overall and, in many regions, identical. Due to the degeneracy of the genetic code, differences between the former and latter nucleotide sequences may be silent (i.e., the amino acids encoded by the polynucleotide are the same, and the variant polynucleotide sequence encodes the same amino acid sequence as the presently disclosed polynucleotide. Variant nucleotide sequences may encode different amino acid sequences, in which case such nucleotide differences will result in amino acid substitutions, additions, deletions, insertions, truncations or fusions with respect to the similar disclosed polynucleotide sequences. These variations result in polynucleotide variants encoding polypeptides that share at least one functional characteristic. The degeneracy of the genetic code also dictates that many different variant polynucleotides can encode identical and/or substantially similar polypeptides in addition to those sequences illustrated in the Sequence Listing.

Also within the scope of the invention is a variant of a transcription factor nucleic acid listed in the Sequence Listing, that is, one having a sequence that differs from the one of the polynucleotide sequences in the Sequence Listing, or a complementary sequence, that encodes a functionally equivalent polypeptide (i.e., a polypeptide having some degree of equivalent or similar biological activity) but differs in sequence from the sequence in the Sequence Listing, due to degeneracy in the genetic code. Included within this definition are polymorphisms that may or may not be readily detectable using a particular oligonucleotide probe of the polynucleotide encoding polypeptide, and improper or unexpected hybridization to allelic variants, with a locus other than the normal chromosomal locus for the polynucleotide sequence encoding polypeptide.

“Allelic variant” or “polynucleotide allelic variant” refers to any of two or more alternative forms of a gene occupying the same chromosomal locus. Allelic variation arises naturally through mutation, and may result in phenotypic polymorphism within populations. Gene mutations may be “silent” or may encode polypeptides having altered amino acid sequence. “Allelic variant” and “polypeptide allelic variant” may also be used with respect to polypeptides, and in this case the term refer to a polypeptide encoded by an allelic variant of a gene.

“Splice variant” or “polynucleotide splice variant” as used herein refers to alternative forms of RNA transcribed from a gene. Splice variation naturally occurs as a result of alternative sites being spliced within a single transcribed RNA molecule or between separately transcribed RNA molecules, and may result in several different forms of mRNA transcribed from the same gene. Thus, splice variants may encode polypeptides having different amino acid sequences, which may or may not have similar functions in the organism. “Splice variant” or “polypeptide splice variant” may also refer to a polypeptide encoded by a splice variant of a transcribed mRNA.

As used herein, “polynucleotide variants” may also refer to polynucleotide sequences that encode paralogs and orthologs of the presently disclosed polypeptide sequences. “Polypeptide variants” may refer to polypeptide sequences that are paralogs and orthologs of the presently disclosed polypeptide sequences.

Differences between presently disclosed polypeptides and polypeptide variants are limited so that the sequences of the former and the latter are closely similar overall and, in many regions, identical. Presently disclosed polypeptide sequences and similar polypeptide variants may differ in amino acid sequence by one or more substitutions, additions, deletions, fusions and truncations, which may be present in any combination. These differences may produce silent changes and result in a functionally equivalent transcription factor. Thus, it will be readily appreciated by those of skill in the art, that any of a variety of polynucleotide sequences is capable of encoding the transcription factors and transcription factor homolog polypeptides of the invention. A polypeptide sequence variant may have “conservative” changes, wherein a substituted amino acid has similar structural or chemical properties. Deliberate amino acid substitutions may thus be made on the basis of similarity in polarity, charge, solubility, hydrophobicity, hydrophilicity, and/or the amphipathic nature of the residues, as long as the functional or biological activity of the transcription factor is retained. For example, negatively charged amino acids may include aspartic acid and glutamic acid, positively charged amino acids may include lysine and arginine, and amino acids with uncharged polar head groups having similar hydrophilicity values may include leucine, isoleucine, and valine; glycine and alanine; asparagine and glutamine; serine and threonine; and phenylalanine and tyrosine. More rarely, a variant may have “non-conservative” changes, e.g., replacement of a glycine with a tryptophan. Similar minor variations may also include amino acid deletions or insertions, or both. Related polypeptides may comprise, for example, additions and/or deletions of one or more N-linked or O-linked glycosylation sites, or an addition and/or a deletion of one or more cysteine residues. Guidance in determining which and how many amino acid residues may be substituted, inserted or deleted without abolishing functional or biological activity may be found using computer programs well known in the art, for example, DNASTAR software (U.S. Pat. No. 5,840,544).

“Fragment”, with respect to a polynucleotide, refers to a clone or any part of a polynucleotide molecule that retains a usable, functional characteristic. Useful fragments include oligonucleotides and polynucleotides that may be used in hybridization or amplification technologies or in the regulation of replication, transcription or translation. A “polynucleotide fragment” refers to any subsequence of a polynucleotide, typically, of at least about nine consecutive nucleotides, preferably at least about 30 nucleotides, more preferably at least about 50 nucleotides, of any of the sequences provided herein. Exemplary polynucleotide fragments are the first sixty consecutive nucleotides of the transcription factor polynucleotides listed in the Sequence Listing. Exemplary fragments also include fragments that comprise a region that encodes an AP2 domain of a transcription factor. Exemplary fragments also include fragments that comprise a conserved domain of a transcription factor. Exemplary fragments include fragments that comprise an AP2 conserved domain, for example, amino acid residues 16-80 of G1792 (SEQ ID NO: 2), or an EDLL domain (SEQ ID NO: 63), amino acid residues 117-132, as noted in Table 1.

Fragments may also include subsequences of polypeptides and protein molecules, or a subsequence of the polypeptide. Fragments may have uses in that they may have antigenic potential. In some cases, the fragment or domain is a subsequence of the polypeptide which performs at least one biological function of the intact polypeptide in substantially the same manner, or to a similar extent, as does the intact polypeptide. For example, a polypeptide fragment can comprise a recognizable structural motif or functional domain such as a DNA-binding site or domain that binds to a DNA promoter region, an activation domain, or a domain for protein-protein interactions, and may initiate transcription. Fragments can vary in size from as few as 3 amino acid residues to the full length of the intact polypeptide, but are preferably at least about 30 amino acid residues in length and more preferably at least about 60 amino acid residues in length.

The invention also encompasses production of DNA sequences that encode transcription factors and transcription factor derivatives, or fragments thereof, entirely by synthetic chemistry. After production, the synthetic sequence may be inserted into any of the many available expression vectors and cell systems using reagents well known in the art. Moreover, synthetic chemistry may be used to introduce mutations into a sequence encoding transcription factors or any fragment thereof.

“Derivative” refers to the chemical modification of a nucleic acid molecule or amino acid sequence. Chemical modifications can include replacement of hydrogen by an alkyl, acyl, or amino group or glycosylation, pegylation, or any similar process that retains or enhances biological activity or lifespan of the molecule or sequence.

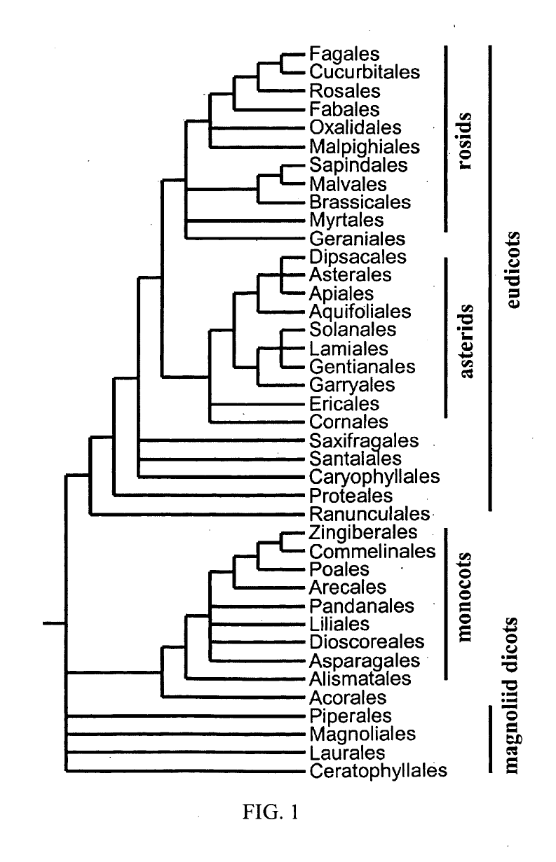

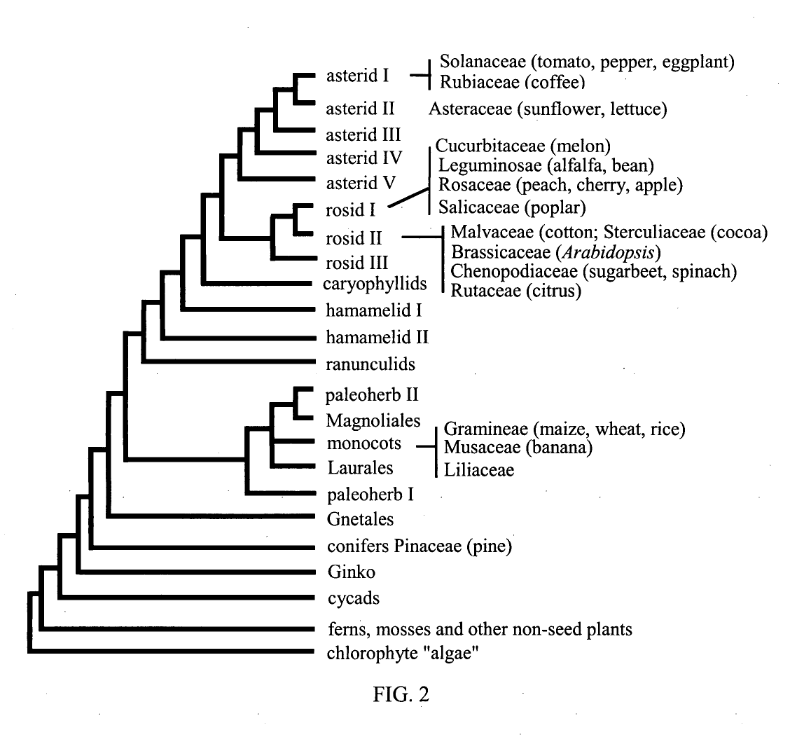

The term “plant” includes whole plants, shoot vegetative organs/structures (for example, leaves, stems and tubers), roots, flowers and floral organs/structures (for example, bracts, sepals, petals, stamens, carpels, anthers and ovules), seed (including embryo, endosperm, and seed coat), fruit (the mature ovary), plant tissue (for example, vascular tissue or ground tissue), cells (for example, guard cells, egg cells, and the like), and progeny of plants. The class of plants that can be used in the method of the invention is generally as broad as the class of higher and lower plants amenable to transformation techniques, including angiosperms (monocotyledonous and dicotyledonous plants), gymnosperms, ferns, horsetails, psilophytes, lycophytes, bryophytes, and multicellular algae (as shown in FIG. 1, adapted from Daly et al. (2001) Plant Physiol. 127: 1328-1333; FIG. 2, adapted from Ku et al. (2000) Proc. Natl. Acad. Sci. USA 97: 9121-9126; and also Tudge in The Variety of Life, Oxford University Press, New York, N.Y. (2000) pp. 547-606).

A “transgenic plant” refers to a plant that contains genetic material not found in a wild-type plant of the same species, variety or cultivar. The genetic material may include a transgene, an insertional mutagenesis event (such as by transposon or T-DNA insertional mutagenesis), an activation tagging sequence, a mutated sequence, a homologous recombination event or a sequence modified by chimeraplasty. Typically, the foreign genetic material has been introduced into the plant by human manipulation, but any method can be used as one of skill in the art recognizes.

A transgenic plant may contain an expression vector or cassette. The expression cassette typically comprises a polypeptide-encoding sequence operably linked (i.e., under regulatory control of) to appropriate inducible or constitutive regulatory sequences that allow for the expression of polypeptide. The expression cassette can be introduced into a plant by transformation or by breeding after transformation of a parent plant. A plant refers to a whole plant as well as to a plant part, such as seed, fruit, leaf, or root, plant tissue, plant cells or any other plant material, e.g., a plant explant, as well as to progeny thereof, and to in vitro systems that mimic biochemical or cellular components or processes in a cell.

“Wild type” or “wild-type”, as used herein, refers to a plant cell, seed, plant component, plant tissue, plant organ or whole plant that has not been genetically modified or treated in an experimental sense. Wild-type cells, seed, components, tissue, organs or whole plants may be used as controls to compare levels of expression and the extent and nature of trait modification with cells, tissue or plants of the same species in which a transcription factor expression is altered, e.g., in that it has been knocked out, overexpressed, or ectopically expressed.

A “control plant” as used in the present invention refers to a plant cell, seed, plant component, plant tissue, plant organ or whole plant used to compare against transgenic or genetically modified plant for the purpose of identifying an enhanced phenotype in the transgenic or genetically modified plant. A control plant may in some cases be a transgenic plant line that comprises an empty vector or marker gene, but does not contain the recombinant polynucleotide of the present invention that is expressed in the transgenic or genetically modified plant being evaluated. In general, a control plant is a plant of the same line or variety as the transgenic or genetically modified plant being tested. A suitable control plant would include a genetically unaltered or non-transgenic plant of the parental line used to generate a transgenic plant herein.

A “trait” refers to a physiological, morphological, biochemical, or physical characteristic of a plant or particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring tolerance to water deprivation or particular salt or sugar concentrations, or by the observation of the expression level of a gene or genes, e.g., by employing Northern analysis, RT-PCR, microarray gene expression assays, or reporter gene expression systems, or by agricultural observations such as osmotic stress tolerance or yield. Any technique can be used to measure the amount of, comparative level of, or difference in any selected chemical compound or macromolecule in the transgenic plants, however.

“Trait modification” refers to a detectable difference in a characteristic in a plant ectopically expressing a polynucleotide or polypeptide of the present invention relative to a plant not doing so, such as a wild-type plant. In some cases, the trait modification can be evaluated quantitatively. For example, the trait modification can entail at least about a 2% or greater increase or decrease in an observed trait compared with a wild-type or control plant. It is known that there can be a natural variation in the modified trait. Therefore, the trait modification observed entails a change of the normal distribution of the trait in the plants compared with the distribution observed in wild-type plants.

When two or more plants are “morphologically similar” they have comparable forms or appearances, including analogous features such as dimension, height, width, mass, root mass, shape, glossiness, color, stem diameter, leaf size, leaf dimension, leaf density, internode distance, branching, root branching, number and form of inflorescences, and other macroscopic characteristics. “Developmentally similar” plants generally progress through their life cycles at approximately the same rates. Plant characteristics falling with the natural range of variations observed in a given environment may be considered similar.

“Modulates” refers to a change in activity (biological, chemical, or immunological) or lifespan resulting from specific binding between a molecule and either a nucleic acid molecule or a protein.

The term “transcript profile” refers to the expression levels of a set of genes in a cell in a particular state, particularly by comparison with the expression levels of that same set of genes in a cell of the same type in a reference state. For example, the transcript profile of a particular transcription factor in a suspension cell is the expression levels of a set of genes in a cell knocking out or overexpressing that transcription factor compared with the expression levels of that same set of genes in a suspension cell that has normal levels of that transcription factor. The transcript profile can be presented as a list of those genes whose expression level is significantly different between the two treatments, and the difference ratios. Differences and similarities between expression levels may also be evaluated and calculated using statistical and clustering methods.

“Ectopic expression or altered expression” in reference to a polynucleotide indicates that the pattern of expression in, e.g., a transgenic plant or plant tissue, is different from the expression pattern in a wild-type plant or a reference plant of the same species. The pattern of expression may also be compared with a reference expression pattern in a wild-type plant of the same species. For example, the polynucleotide or polypeptide is expressed in a cell or tissue type other than a cell or tissue type in which the sequence is expressed in the wild-type plant, or by expression at a time other than at the time the sequence is expressed in the wild-type plant, or by a response to different inducible agents, such as hormones or environmental signals, or at different expression levels (either higher or lower) compared with those found in a wild-type plant. The term also refers to altered expression patterns that are produced by lowering the levels of expression to below the detection level or completely abolishing expression. The resulting expression pattern can be transient or stable, constitutive or inducible. In reference to a polypeptide, the term “ectopic expression or altered expression” further may relate to altered activity levels resulting from the interactions of the polypeptides with exogenous or endogenous modulators or from interactions with factors or as a result of the chemical modification of the polypeptides.

The term “overexpression” as used herein refers to a greater expression level of a gene in a plant, plant cell or plant tissue, compared to expression in a wild-type plant, cell or tissue, at any developmental or temporal stage for the gene. Overexpression can occur when, for example, the genes encoding one or more transcription factors are under the control of a strong expression signal, such as one of the promoters described herein (e.g., the cauliflower mosaic virus 35S transcription initiation region). Overexpression may occur throughout a plant or in specific tissues of the plant, depending on the promoter used, as described below.

Overexpression may take place in plant cells normally lacking expression of polypeptides functionally equivalent or identical to the present transcription factors. Overexpression may also occur in plant cells where endogenous expression of the present transcription factors or functionally equivalent molecules normally occurs, but such normal expression is at a lower level. Overexpression thus results in a greater than normal production, or “overproduction” of the transcription factor in the plant, cell or tissue.

The term “transcription regulating region” refers to a DNA regulatory sequence that regulates expression of one or more genes in a plant when a transcription factor having one or more specific binding domains binds to the DNA regulatory sequence. Transcription factors of the present invention possess an AP2 domain. Examples of AP2 or EDLL conserved domains of the sequences of the invention may be found in Table 1. The transcription factors of the invention also comprise an amino acid subsequence that forms a transcription activation domain that regulates expression of one or more abiotic stress or low nitrogen tolerance genes in a plant when the transcription factor binds to the regulating region.

“Substantially purified” refers to nucleic acid molecules or proteins that are removed from their natural environment and are isolated or separated, and are at least about 60% free, preferably about 75% free, and most preferably about 90% free, from other components with which they are naturally associated.

### DESCRIPTION OF THE SPECIFIC EMBODIMENTS

Transcription Factors Modify Expression of Endogenous Genes

A transcription factor may include, but is not limited to, any polypeptide that can activate or repress transcription of a single gene or a number of genes. As one of ordinary skill in the art recognizes, transcription factors can be identified by the presence of a region or domain of structural similarity or identity to a specific consensus sequence or the presence of a specific consensus DNA-binding site or DNA-binding site motif (for example, Riechmann et al. (2000) supra). The plant transcription factors of the present invention belong to the AP2 transcription factor family (Riechmann and Meyerowitz (1998) Biol. Chem. 379: 633-646).

Generally, the transcription factors encoded by the present sequences are involved in cell differentiation and proliferation and the regulation of growth. Accordingly, one skilled in the art would recognize that by expressing the present sequences in a plant, one may change the expression of autologous genes or induce the expression of introduced genes. By affecting the expression of similar autologous sequences in a plant that have the biological activity of the present sequences, or by introducing the present sequences into a plant, one may alter a plant's phenotype to one with improved traits related to osmotic stresses. The sequences of the invention may also be used to transform a plant and introduce desirable traits not found in the wild-type cultivar or strain. Plants may then be selected for those that produce the most desirable degree of over- or under-expression of target genes of interest and coincident trait improvement.

The sequences of the present invention may be from any species, particularly plant species, in a naturally occurring form or from natural, synthetic, semi-synthetic or recombinant source. Sequences of the invention may also include fragments of present amino acid sequences. Where “amino acid sequence” is recited to refer to an amino acid sequence of a naturally occurring protein molecule, “amino acid sequence” and like terms are not meant to limit the amino acid sequence to the complete native sequence associated with the recited protein molecule.

In addition to methods for modifying a plant phenotype by employing one or more polynucleotides and polypeptides of the invention described herein, the polynucleotides and polypeptides of the invention have a variety of additional uses. These uses include their use in the recombinant production (i.e., expression) of proteins; as regulators of plant gene expression, as diagnostic probes for the presence of complementary or partially complementary nucleic acids (including for detection of natural coding nucleic acids); as substrates for further reactions, e.g., mutation reactions, PCR reactions, or the like; as substrates for cloning e.g., including digestion or ligation reactions; and for identifying exogenous or endogenous modulators of the transcription factors. In many instances, a polynucleotide comprises a nucleotide sequence encoding a polypeptide (or protein) or a domain or fragment thereof. Additionally, the polynucleotide may comprise a promoter, an intron, an enhancer region, a polyadenylation site, a translation initiation site, 5′ or 3′ untranslated regions, a reporter gene, a selectable marker, or the like. The polynucleotide can be single stranded or double stranded DNA or RNA. The polynucleotide optionally comprises modified bases or a modified backbone. The polynucleotide can be, e.g., genomic DNA or RNA, a transcript (such as an mRNA), a cDNA, a PCR product, a cloned DNA, a synthetic DNA or RNA, or the like. The polynucleotide can comprise a sequence in either sense or antisense orientations.

Expression of genes that encode transcription factors that modify expression of endogenous genes, polynucleotides, and proteins are well known in the art. In addition, transgenic plants comprising isolated polynucleotides encoding transcription factors may also modify expression of endogenous genes, polynucleotides, and proteins. Examples include Peng et al. (1997) Genes Development 11: 3194-3205, and Peng et al. (1999) Nature, 400: 256-261. In addition, many others have demonstrated that an Arabidopsis transcription factor expressed in an exogenous plant species elicits the same or very similar phenotypic response (for example, Fu et al. (2001) Plant Cell 13: 1791-1802; Nandi et al. (2000) Curr. Biol. 10: 215-218; Coupland (1995) Nature 377: 482-483; and Weigel and Nilsson (1995) Nature 377: 482-500).

In another example, a transcription factor expressed in another plant species elicits the same or very similar phenotypic response of the endogenous sequence, as often predicted in earlier studies of Arabidopsis transcription factors in Arabidopsis (Mandel et al. (1992) Cell 71-133-143) and Suzuki et al. (2001) Plant J. 28: 409-418). Other examples include Müller et al. (2001) Plant J. 28: 169-179; Kim et al. (2001) Plant J. 25: 247-259; Kyozuka and Shimamoto (2002) Plant Cell Physiol. 43: 130-135; Boss and Thomas (2002) Nature, 416: 847-850; He et al. (2000) Transgenic Res. 9: 223-227; and Robson et al. (2001) Plant J. 28: 619-631.

In yet another example, Gilmour et al. ((1998) Plant J. 16: 433-442) teach an Arabidopsis AP2 transcription factor, CBF1, that increases plant freezing tolerance when overexpressed in transgenic plants. Jaglo et al. ((2001) Plant Physiol. 127: 910-917) further identified sequences in Brassica napus which encode CBF-like genes and that transcripts for these genes accumulated rapidly in response to low temperature. Transcripts encoding CBF-like proteins were also found to accumulate rapidly in response to low temperature in wheat, as well as in tomato. An alignment of the CBF proteins from Arabidopsis, B. napus, wheat, rye, and tomato revealed the presence of conserved consecutive amino acid residues, PKK/RPAGRxKFxETRHP and DSAWR, which bracket the AP2/EREBP DNA binding domains of the proteins and distinguish them from other members of the AP2/EREBP protein family (Jaglo et al. (2001) supra).

Transcription factors mediate cellular responses and control traits through altered expression of genes containing cis-acting nucleotide sequences that are targets of the introduced transcription factor. It is well appreciated in the Art that the effect of a transcription factor on cellular responses or a cellular trait is determined by the particular genes whose expression is either directly or indirectly (e.g., by a cascade of transcription factor binding events and transcriptional changes) altered by transcription factor binding. In a global analysis of transcription comparing a standard condition with one in which a transcription factor is overexpressed, the resulting transcript profile associated with transcription factor overexpression is related to the trait or cellular process controlled by that transcription factor. For example, the PAP2 gene and other genes in the MYB family have been shown to control anthocyanin biosynthesis through regulation of the expression of genes known to be involved in the anthocyanin biosynthetic pathway (Bruce et al. (2000) Plant Cell 12: 65-79; and Borevitz et al. (2000) Plant Cell 12: 2383-2393). Further, global transcript profiles have been used successfully as diagnostic tools for specific cellular states (e.g., cancerous vs. non-cancerous; Bhattacharjee et al. (2001) Proc. Natl. Acad. Sci. USA 98: 13790-13795; and Xu et al. (2001) Proc. Natl. Acad. Sci. USA 98: 15089-15094). Consequently, it is evident to one skilled in the art that similarity of transcript profile upon overexpression of different transcription factors would indicate similarity of transcription factor function.

Polypeptides and Polynucleotides of the Invention. The present invention provides, among other things, transcription factors (TFs), and transcription factor homolog polypeptides, and isolated or recombinant polynucleotides encoding the polypeptides, or novel sequence variant polypeptides or polynucleotides encoding novel variants of transcription factors derived from the specific sequences provided in the Sequence Listing. Also provided are methods for increasing a plant's tolerance to one or conditions of abiotic stress, including low nitrogen, cold, heat, or hyperosmotic stress such as high salt or drought. These methods are based on the ability to alter the expression of critical regulatory molecules that may be conserved between diverse plant species. Related conserved regulatory molecules may be originally discovered in a model system such as Arabidopsis and homologous, functional molecules then discovered in other plant species. The latter may then be used to confer tolerance to one or more abiotic stresses, including low nitrogen, high salt, drought, heat and/or cold, in diverse plant species.

Exemplary polynucleotides encoding polypeptides of the invention were identified in the Arabidopsis thaliana GenBank database using publicly available sequence analysis programs and parameters. Sequences initially identified were characterized to identify sequences comprising specified sequence strings corresponding to motifs present in families of known transcription factors. In addition, further exemplary polynucleotides encoding the polypeptides of the invention were identified in the plant GenBank database using publicly available sequence analysis programs and parameters. Sequences initially identified were then further characterized to identify sequences comprising specified sequence strings corresponding to sequence motifs present in families of known transcription factors. Polynucleotide sequences meeting such criteria were confirmed as transcription factors.

Additional polynucleotides of the invention were identified by screening Arabidopsis thaliana and/or other plant cDNA libraries with probes corresponding to known transcription factors under low stringency hybridization conditions. Additional sequences, including full length coding sequences were subsequently recovered by the rapid amplification of cDNA ends (RACE) procedure, using a commercially available kit according to the manufacturer's instructions. Where necessary, multiple rounds of RACE are performed to isolate 5′ and 3′ ends. The full-length cDNA was then recovered by a routine end-to-end polymerase chain reaction (PCR) using primers specific to the isolated 5′ and 3′ ends. Exemplary sequences are provided in the Sequence Listing.

These sequences and others derived from diverse species and found in the sequence listing have been ectopically expressed in overexpressor plants. The changes in the characteristic(s) or trait(s) of the plants were then observed and found to confer increased abiotic stress or low nitrogen tolerance. Therefore, the polynucleotides and polypeptides can be used to improve desirable characteristics of plants.

The polynucleotides of the invention were also ectopically expressed in overexpressor plant cells and the changes in the expression levels of a number of genes, polynucleotides, and/or proteins of the plant cells observed. Therefore, the polynucleotides and polypeptides can be used to change expression levels of a genes, polynucleotides, and/or proteins of plants.

The AP2 family, including the G1792 clade. AP2 (APETALA2) and EREBPs (Ethylene-Responsive Element Binding Proteins) are the prototypic members of a family of transcription factors unique to plants, whose distinguishing characteristic is that they contain AP2 DNA-binding domain (a review appears in Riechmann and Meyerowitz (1998) Biol. Chem. 379: 633-646). The AP2 domain was first recognized as a repeated motif within the Arabidopsis thaliana AP2 protein (Jofuku et al. (1994) Plant Cell 6: 1211-1225). Shortly afterwards, four DNA-binding proteins from tobacco were identified that interact with a sequence that is essential for the responsiveness of some promoters to the plant hormone ethylene, and were designated as ethylene-responsive element binding proteins (EREBPs; Ohme-Takagi et al. (1995) Plant Cell 7: 173-182). The DNA-binding domain of EREBP-2 was mapped to a region that was common to all four proteins (Ohme-Takagi et al (1995) supra), and that was found to be closely related to the AP2 domain (Weigel (1995) Plant Cell 7: 388-389) but that did not bear sequence similarity to previously known DNA-binding motifs.

AP2/EREBP genes form a large family, with many members known in several plant species (Okamuro et al. (1997) Proc. Natl. Acad. Sci. USA 94: 7076-7081; Riechmann and Meyerowitz (1998) supra). The number of AP2/EREBP genes in the Arabidopsis thaliana genome is approximately 145 (Riechmann et al. (2000) Science 290: 2105-2110). The APETALA2 class is characterized by the presence of two AP2 DNA binding domains, and contains 14 genes. The AP2/ERF is the largest subfamily, and includes 125 genes which are involved in abiotic (DREB subgroup) and biotic (ERF subgroup) stress responses and the RAV subgroup includes 6 genes which all have a B3 DNA binding domain in addition to the AP2 DNA binding domain (Kagaya et al. (1999) Nucleic Acids Res. 27: 470-478).

Arabidopsis AP2 is involved in the specification of sepal and petal identity through its activity as a homeotic gene that forms part of the combinatorial genetic mechanism of floral organ identity determination and it is also required for normal ovule and seed development (Bowman et al. (1991) Development 112: 1-20; Jofuku et al. (1994) supra). Arabidopsis ANT is required for ovule development and it also plays a role in floral organ growth (Elliott et al. (1996) Plant Cell 8: 155-168; Klucher et al. (1996) Plant Cell 8: 137-153). Finally, maize G115 regulates leaf epidermal cell identity (Moose et al. (1996) Genes Dev. 10: 3018-3027).

The attack of a plant by a pathogen may induce defense responses that lead to resistance to the invasion, and these responses are associated with transcriptional activation of defense-related genes, among them those encoding pathogenesis-related (PR) proteins. The involvement of EREBP-like genes in controlling the plant defense response is based on the observation that many PR gene promoters contain a short cis-acting element that mediates their responsiveness to ethylene (ethylene appears to be one of several signal molecules controlling the activation of defense responses). Tobacco EREBP-1, -2, -3, and -4, and tomato Pti4, Pti5 and Pti6 proteins have been shown to recognize such cis-acting elements (Ohme-Takagi (1995) supra; Zhou et al. (1997) EMBO J. 16: 3207-3218). In addition, Pti4, Pti5, and Pti6 proteins have been shown to directly interact with Pto, a protein kinase that confers resistance against Pseudomonas syringae pv tomato (Zhou et al. (1997) supra). Plants are also challenged by adverse environmental conditions like cold or drought, and EREBP-like proteins appear to be involved in the responses to these abiotic stresses as well. COR (for cold-regulated) gene expression is induced during cold acclimation, the process by which plants increase their resistance to freezing in response to low unfreezing temperatures. The Arabidopsis EREBP-like gene CBF1 (Stockinger et al. (1997) Proc. Natl. Acad. Sci. USA 94: 1035-1040) is a regulator of the cold acclimation response, because ectopic expression of CBF1 in Arabidopsis transgenic plants induced COR gene expression in the absence of a cold stimulus, and the plant freezing tolerance was increased (Jaglo-Ottosen et al. (1998) Science 280: 104-106). Finally, another Arabidopsis EREBP-like gene, ABI4, is involved in abscisic acid (ABA) signal transduction, because abi4 mutants are insensitive to ABA (ABA is a plant hormone that regulates many agronomically important aspects of plant development; Finkelstein et al. (1998) Plant Cell 10: 1043-1054).

We first identified G1792 (AT3G23230) as a putative transcription factor in the sequence of BAC clone K14B15 (AB025608, gene K14B15.14). We have assigned the name TRANSCRIPTIONAL REGULATOR OF DEFENSE RESPONSE 1 (TDR1) to this gene, based on its apparent role in disease responses. The G1792 protein and other polypeptides within the G1792 clade contain a single AP2 domain and belong to the ERF class of AP2 proteins.

The primary amino acid sequence of G1792 and other members of the G1792 clade, showing the relative positions of the AP2 domain, are presented in FIGS. 3A-3L. In addition to the AP2 domain, the G1792 clade of transcription factor polypeptides contains a putative activation domain designated the “EDLL domain”. Four amino acids are highly conserved in the paralogs and orthologs of G1792 within this domain. These conserved residues comprise glutamic acid, aspartic acid, and two leucine residues (hence the “EDLL” designation) in the subsequence:

Glu-(Xaa)₄-Asp-(Xaa)₃-Leu-(Xaa)₃-Leu (SEQ ID NO: 63)

where Xaa can be any amino acid, including those represented in FIG. 4.

AtERF type transcription factors respond to abiotic stress. While ERF type transcription factors are primarily recognized for responding to a variety of biotic stresses (such as pathogen infection), some ERFs have been characterized as being responsive to abiotic stress. Fujimoto et. al. (2000) Plant Cell 12: 393-404 have shown that AtERF1-5, corresponding to G28 (SEQ ID NO: 48), G1006 (SEQ ID NO: 46), G1005 (SEQ ID NO: 62), G6 (SEQ ID NO: 58), and G1004 (SEQ ID NO: 60), respectively, can respond to various abiotic stresses, including cold, heat, drought, ABA, CHX, and wounding. Genes normally associated with the plant defense response (PR1, PR2, PR5, and peroxidases) have also been shown to be regulated by water stress (Zhu et. al. (1995) Plant Physiol. 108: 929-937; Ingram and Bartels (1996). Annu Rev. Plant Physiol. Plant Mol. Biol. 47:377-403) suggesting some overlap between the two responses. A target sequence for ERF-type transcription factors has been identified and extensively studied (Hao et al. (1998) J. Biol. Chem. 273: 26857-26861). This target sequence consists of AGCCGCC and has been found in the 5′ upstream regions of genes responding to disease and regulated by ERFs. However, it is also certainly the case that several genes (ARSK1 and dehydrin) known to be induced by ABA, NaCl, cold and wounding, also possess a GCC box regulatory element in their 5′ upstream regions (Hwang and Goodman (1995) Plant J. 8: 37-43) suggesting that ERF type transcription factors may regulate also regulate abiotic stress associated genes.

ERF type transcription factors in other species. ERF-type transcription factors are well known to be transcriptional activators of disease responses (Fujimoto et. al. (2000) supra; Gu et al. (2000) Plant Cell 12: 771-786; Chen et al. (2002) Plant Cell 14: 559-574; Cheong et al. (2002) Plant Physiol. 129: 661-677; Onate-Sanchez and Singh (2002) Plant Physiol. 128: 1313-1322; Brown et al. (2003) Plant Physiol. 132: 1020-1032; Lorenzo et al. (2003) Plant Cell 15: 165-178) but have not been well characterized as being involved in response to abiotic stress conditions such as drought. Other AP2 transcription factors (DREBs), including the CBF class, are known to bind DRE elements in genes responding to abiotic stresses such as drought, high salt, and cold. (Haake et al. (2002) Plant Physiol. 130: 639-648; Thomashow (2001) Plant Physiol. 125: 89-93, Liu et al. (1998) Plant Cell 10: 1391-1406; Gilmour et al. (2000) Plant Physiol. 124: 1854-1865; and Shinozaki and Yamaguchi-Shinozaki (2000) Curr. Opin. Plant Biol. 3: 217-223).

The role of ERF type transcription factors in disease responses. Pti4, Pti5 and Pti6 were identified as interactors with the tomato disease resistance protein Pto in yeast 2-hybrid assays (Zhou et al, (1997) EMBO J. 16: 3207-3218). Since that time, several ERF genes have been shown to enhance disease resistance when overexpressed in Arabidopsis or other species. These ERF genes include ERF1 (G1266) of Arabidopsis (Berrocal-Lobo et al. (2002) Plant J. 29: 23-32, Pti4 (Gu et al. (2002) Plant Cell 14: 817-831 and Pti5 (He et al. (2001) Mol. Plant. Microbe Interact. 14: 1453-1457) of tomato, Tsi1 of tobacco (Park et. al. (2001) supra; Shin et al. (2002) Mol. Plant Microbe Interact. 15: 983-989, and AtERF1 (G28, SEQ ID NO: 48) and TDR1 (G1792, SEQ ID NO: 2) of Arabidopsis (included in the present data).

Regulation of ERF TFs by pathogen and small molecule signaling. ERF genes show a variety of stress-regulated expression patterns. Regulation by disease-related stimuli such as ethylene (ET), jasmonic acid (JA), salicylic acid (SA), and infection by virulent or avirulent pathogens has been shown for a number of ERF genes (Fujimoto et. al. (2000) supra; Gu et al. (2000) supra; Chen et al. (2002) supra; Cheong et al. (2002) supra; Onate-Sanchez and Singh (2002) supra; Brown et al. (2003) supra; Lorenzo et al. (2003) supra). However, some ERF genes are also induced by wounding and abiotic stresses (Fujimoto et. al. (2000) supra; Park et al. (2001) Plant Cell 13: 1035-1046; Chen et al. (2002) supra; Tournier et al. (2003) FEBS Lett. 550: 149-154). Currently, it is difficult to assess the overall picture of ERF regulation in relation to phylogeny, since different studies have concentrated on different ERF genes, treatments and time points. Significantly, several ERF transcription factors that confer enhanced disease resistance when overexpressed, such as ERF1, Pti4, and AtERF1, are transcriptionally regulated by pathogens, ET, and JA (Fujimoto et. al. (2000) supra; Onate-Sanchez and Singh (2002) supra; Brown et al. (2003) supra; Lorenzo et al. (2003) supra). ERF1 is induced synergistically by ET and JA, and induction by either hormone is dependent on an intact signal transduction pathway for both hormones, indicating that ERF1 may be a point of integration for ET and JA (Lorenzo et al. (2003) supra). At least 4 other ERFs are also induced by JA and ET (Brown et al. (2003) supra), implying that other ERFs are probably also important in ET/JA signal transduction. A number of the genes in subgroup 1, including AtERF3 and AtERF4, are thought to act as transcriptional repressors (Fujimoto et. al. (2000) supra), and these two genes were found to be induced by ET, JA, and an incompatible pathogen (Brown et al. (2003) supra).

The SA signal transduction pathway can act antagonistically to the ET/JA pathway. Interestingly, Pti4 and AtERF1 are induced by SA as well as by JA and ET (Gu et al. (2000) supra; Onate-Sanchez and Singh (2002) supra). Pti4, Pti5 and Pti6 have been implicated indirectly in regulation of the SA response, perhaps through interaction with other transcription factors, since overexpression of these genes in Arabidopsis induced SA-regulated genes without SA treatment and enhanced the induction seen after SA treatment (Gu et al. (2002) supra).

Post-transcriptional regulation of ERF genes by phosphorylation may be a significant form of regulation. Pti4 has been shown to be phosphorylated specifically by the Pto kinase, and this phosphorylation enhances binding to its target sequence (Gu et al. (2000) supra). Recently, the OsEREBP1 gene of rice has been shown to be phosphorylated by the pathogen-induced MAP kinase BWMK1, and this phosphorylation was shown to enhance its binding to the GCC box (Cheong et al. (2003) Plant Physiol. 132: 1961-1972), suggesting that phosphorylation of ERF proteins may be a common theme. A potential MAPK phosphorylation site has been noted in AtERF5 (Fujimoto et. al. (2000) supra).

Target genes regulated by ERF TFs. Binding of ERF transcription factors to the target sequence AGCCGCC (the GCC box) has been extensively studied (Hao et al. (1998) supra). This element is found in a number of promoters of pathogenesis-related and ET- or JA-induced genes. However, it is unclear how much overlap there is in target genes for particular ERFs. Recent studies have profiled genes induced in Arabidopsis plants overexpressing ERF1 (Lorenzo et al. (2003) supra) and Pti4 (Chakravarthy et al. (2003) Plant Cell 15: 3033-3050). However, these studies were done with different technology (Affymetrix GeneChip vs. serial analysis of gene expression) and under different conditions, and it is therefore difficult to compare the results directly. There is evidence that flanking sequences can affect the binding of ERFs to the GCC box (Gu et al. (2002) supra; Tournier et al. (2003) supra), so it is likely that different ERFs will regulate somewhat different gene sets.

Protein structure and properties: tertiary structure. The solution structure of an ERF type transcription factor domain in complex with the GCC box has been determined (Allen et. al. (1998) EMBO J. 17: 5484-5496). It consists of a β-sheet composed of three strands and an α-helix. Flanking sequences of the AP2 domain of this protein were replaced with the flanking sequences of the related CBF1 protein and the chimeric protein was found to contain the same arrangement of secondary structural elements as the native ERF type protein (Allen, M. D., personal communication). This implies that the secondary structural motifs may be conserved for similar ERF type transcription factors within the family.

Protein structure and properties: DNA binding motifs. Two positions have been identified as defining ERF class transcription factors. These consist of amino acids Ala-14 and Asp-19 in the AP2 domain (Sakuma et. al. (2002) Biochem. Biophys. Res. Commun. 290: 998-1009). Recent work indicates that these two amino acids (Ala-14 and Asp-19) have a key function in determining the target specificity (Sakuma et. al. (2002) supra; Hao et al. (2002) Biochemistry 41: 4202-4208) and interact directly with the DNA. The 3-dimensional structure/GCC box complex indicates the interaction of the second strand of the β-sheet with the DNA. The GCC box binding motif of ERF type transcription factors consists of a core sequence of AGCCCGCC.

Table 1 shows the polypeptides identified by: polypeptide SEQ ID NO (first column); the Gene ID (GID) No. and species (second column); the conserved domain coordinates for the AP2 and EDLL domains in amino acid residue coordinates (third column); AP2 domain sequences of the respective polypeptides (fourth column); the identity in percentage terms of the respective AP2 domains to the AP2 domain of G1792 (fifth column); EDLL domain sequences of the respective polypeptides (sixth column); and the percent identity of the respective EDLL domains to the EDLL domain of G1792 (seventh column). The last column shows whether a particular GID under the regulatory control of constitutive or non-constitutive expression systems conferred tolerance or resistance in abiotic stress or disease assays, respectively. Polypeptide sequences that are shown herein to confer low nitrogen or abiotic stress tolerance include Arabidopsis G30, G1791, and G1792, soybean G3518 and G3520, rice G3380, G3381, G3383, G3515, and G3737, and corn G3516 and G3517. These sequences have AP2 domains with 70% or greater identity to the AP2 domain of G1792, and 62% or greater identity to the EDLL domain of G1792.

TABLE 1
Gene families and conserved domains of G1792 clade members

| | | | | | | | Abiotic |
|---|---|---|---|---|---|---|---|
| | | AP2 and EDLL | | % ID | | % ID | stress |
| SEQ | GID | Domains | | to AP2 | | to EDLL | tolerant/ |
| ID | No./ | in AA | | Domain | EDLL | Domain | disease |
| NO: | Species | Coordinates | AP2 domain | of G1792 | Domain | of G1792 | resistant |
| 2 | G1792At | 16-80; | KQARFRGVRRRPWGKFAAEIRDP | 100% | VFEFEYLDD | 100% | +/+ |
| | | 117-132 | SRNGARLWLGTFETAEEAARAYD | | KVLEELL | | |
| | | | RAAFNLRGHLAILNFPNEY | | | | |
| 6 | G1795At | 11-75; | EHGKYRGVRRRPWGKYAAEIRDS | 69% | VFEFEYLDD | 93% | +/+ |
| | | 104-119 | RKHGERVWLGTFDTAEEAARAYD | | SVLEELL | | |
| | | | QAAYSMRGQAAILNFPHEY | | | | |
| 8 | G30At | 16-80; | EQGKYRGVRRRPWGKYAAEIRDS | 70% | VFEFEYLDD | 87% | +/+ |
| | | 100-115 | RKHGERVWLGTFDTAEDAARAYD | | SYLDELL | | |
| | | | RAAYSMRGKAAILNFPHEY | | | | |
| 14 | G3383Os | 9-73; | TATKYRGVRRRPWGKFAAEIRDP | 79% | KIEFEYLDD | 85% | +/wt |
| | | 101-116 | ERGGARVWLGTFDTAEEAARAYD | | KVLDDLL | | |
| | | | RAAYAQRGAAAVLNFPAAA | | | | |
| 4 | G1791At | 10-74; | NEMKYRGVRKRPWGKYAAEIRDS | 73% | VIEFEYLDD | 81% | +/+ |
| | | 108-123 | ARHGARVWLGTFNTAEDAARAYD | | SLLEELL | | |
| | | | RAAFGMRGQRAILNFPHEY | | | | |
| 24 | G3519Gm | 13-77; | CEVRYRGIRRRPWGKFAAEIRDP | 78% | TFELEYLDN | 80% | +/wt |
| | | 128-143 | TRKGTRIWLGTFDTAEQAARAYD | | KLLEELL | | |
| | | | AAAFHFRGHRAILNFPNEY | | | | |
| 12 | G3381Os | 14-78; | LVAKYRGVRRRPWGKFAAEIRDS | 76% | PIEFEYLDD | 78% | +/+ |
| | | 109-124 | SRHGVRVWLGTFDTAEEAARAYD | | HVLQEML | | |
| | | | RSAYSMRGANAVLNFPADA | | | | |
| 32 | G3737Os | 8-72; | AASKYRGVRRRPWGKFAAEIRDP | 76% | KVELVYLD | 78% | +/wt |
| | | 101-116 | ERGGSRVWLGTFDTAEEAARAYD | | DKVLDELL | | |
| | | | RAAFAMKGAMAVLNFPGRT | | | | |
| 16 | G3515Os | 11-75; | SSSSYRGVRKRPWGKFAAEIRDP | 75% | KVELECLDD | 78% | wt/− |
| | | 116-131 | ERGGARVWLGTFDTAEEAARAYD | | KVLEDLL | | |
| | | | RAAFAMKGATAMLNFPGDH | | | | |
| 18 | G3516Zm | 6-70; | KEGKYRGVRKRPWGKFAAEIRDP | 74% | KVELECLDD | 78% | +/wt |
| | | 107-122 | ERGGSRVWLGTFDTAEEAARAYD | | RVLEELL | | |
| | | | RAAFAMKGATAVLNFPASG | | | | |
| 26 | G3520Gm | 14-78; | EEPRYRGVRRRPWGKFAAEIRDP | 80% | VIEFECLDD | 75% | wt/+ |
| | | 109-124 | ARHGARVWLGTFLTAEEAARAYD | | KLLEDLL | | |
| | | | RAAYEMRGALAVLNFPNEY | | | | |
| 20 | G3517Zm | 13-77; | EPTKYRGVRRRPWGKYAAEIRDS | 72% | VIEFEYLDD | 75% | +/+ |
| | | 103-118 | SRHGVRIWLGTFDTAEEAARAYD | | EVLQEML | | |
| | | | RSANSMRGANAVLNFPEDA | | | | |
| 22 | G3518Gm | 13-77; | VEVRYRGIRRRPWGKFAAEIRDP | 78% | TFELEYFDN | 73% | +/nd |
| | | 135-150 | TRKGTRIWLGTFDTAEQAARAYD | | KLLEELL | | |
| | | | AAAFHFRGHRAILNFPNEY | | | | |
| 30 | G3736Ta | 12-76; | EPTKYRGVRRRPWGKFAAEIRDS | 73% | VIEFEYLDD | 68% | nd/nd |
| | | 108-123 | SRHGVRMWLGTFDTAEEAAAAYD | | DVLQSML | | |
| | | | DRSAYSMRGRNAVLNFPDRA | | | | |
| 34 | G3739Zm | 13-77; | EPTKYRGVRRRPWGKYAAEIRDS | 72% | VIELEYLDD | 68% | +/nd |
| | | 107-122 | SRHGVRIWLGTFDTAEEAARAYD | | EVLQEML | | |
| | | | RSAYSMRGANAVLNFPEDA | | | | |
| 28 | G3735Mt | 23-87; | DQIKYRGIRRRPWGKFAAEIRDPT | 78% | ELEFLDNKL | 64% | nd/nd |
| | | 131-144 | RKGTRIWLGTFDTAEQAARAYDAA | | LQELL | | |
| | | | AFHFRGHRAILNFPNEY | | | | |
| 10 | G3380Os | 18-82; | ETTKYRGVRRRPSGKFAAEIRDSS | 77% | VIELECLDD | 62% | +/− |
| | | 103-118 | RQSVRVWLGTFDTAEEAARAYDRA | | QVLQEML | | |
| | | | AYAMRGHLAVLNFPAEA | | | | |
| 36 | G3794Zm | 6-70; | EPTKYRGVRRRPSGKFAAEIRDSS | 73% | VIELECLDD | 62% | +/nd |
| | | 102-117 | RQSVRMWLGTFDTAEEAARAYDRA | | QVLQEML | | |
| | | | AYAMRGQIAVLNFPAEA | | | | |

Abbreviations: At - Arabidopsis thaliana; Gm - Glycine max; Mt - Medicago truncatula Os - Oryza sativa; Ta - Triticum aestivum; Zm - Zea mays wt - wild type nd - not done

The transcription factors of the invention each possess an AP2 domain and an EDLL domain, and include paralogs and orthologs of G1792 found by BLAST analysis, as described below. The AP2 domains of G1792 clade members are at least 69% identical to the AP2 domain of G1792, and the EDLL domains of G1792 clade members are at least 62% identical to the EDLL domain of G1792 (Table 1). These transcription factors rely on the binding specificity and functions of their conserved domains.

Producing Polypeptides. The polynucleotides of the invention include sequences that encode transcription factors and transcription factor homolog polypeptides and sequences complementary thereto, as well as unique fragments of coding sequence, or sequence complementary thereto. Such polynucleotides can be, e.g., DNA or RNA, e.g., mRNA, cRNA, synthetic RNA, genomic DNA, cDNA synthetic DNA, oligonucleotides, etc. The polynucleotides are either double-stranded or single-stranded, and include either, or both sense (i.e., coding) sequences and antisense (i.e., non-coding, complementary) sequences. The polynucleotides include the coding sequence of a transcription factor, or transcription factor homolog polypeptide, in isolation, in combination with additional coding sequences (e.g., a purification tag, a localization signal, as a fusion-protein, as a pre-protein, or the like), in combination with non-coding sequences (e.g., introns or inteins, regulatory elements such as promoters, enhancers, terminators, and the like), and/or in a vector or host environment in which the polynucleotide encoding a transcription factor or transcription factor homolog polypeptide is an endogenous or exogenous gene.

A variety of methods exist for producing the polynucleotides of the invention. Procedures for identifying and isolating DNA clones are well known to those of skill in the art and are described in, e.g., Berger and Kimmel (1987) Guide to Molecular Cloning Techniques, Methods Enzymol. vol. 152, Academic Press, Inc., San Diego, Calif.; Sambrook et al. (1989) supra, vol. 1-3, Cold Spring Harbor Laboratory, Cold Spring Harbor, N.Y., and Ausubel et al. (supplemented through 2000), eds., Current Protocols in Molecular Biology, Greene Publishing Associates, Inc. and John Wiley & Sons, Inc.

Alternatively, polynucleotides of the invention, can be produced by a variety of in vitro amplification methods adapted to the present invention by appropriate selection of specific or degenerate primers. Examples of protocols sufficient to direct persons of skill through in vitro amplification methods, including the polymerase chain reaction (PCR) the ligase chain reaction (LCR), Qβ-replicase amplification and other RNA polymerase mediated techniques (e.g., NASBA), e.g., for the production of the homologous nucleic acids of the invention are found in Berger and Kimmel (1987) supra, Sambrook (1989) supra, and Ausubel (2000) supra, as well as Mullis et al. (1990) PCR Protocols A Guide to Methods and Applications (Innis et al., eds) Academic Press Inc. San Diego, Calif. Improved methods for cloning in vitro amplified nucleic acids are described in Wallace et al. U.S. Pat. No. 5,426,039. Improved methods for amplifying large nucleic acids by PCR are summarized in Cheng et al. (1994) Nature 369: 684-685 and the references cited therein, in which PCR amplicons of up to 40 kb are generated. One of skill will appreciate that essentially any RNA can be converted into a double stranded DNA suitable for restriction digestion, PCR expansion and sequencing using reverse transcriptase and a polymerase (e.g., Ausubel (2000) supra, Sambrook (1989) supra, and Berger and Kimmel (1987) supra).

Alternatively, polynucleotides and oligonucleotides of the invention can be assembled from fragments produced by solid-phase synthesis methods. Typically, fragments of up to approximately 100 bases are individually synthesized and then enzymatically or chemically ligated to produce a desired sequence, e.g., a polynucleotide encoding all or part of a transcription factor. For example, chemical synthesis using the phosphoramidite method is described, e.g., by Beaucage et al. (1981) Tetrahedron Letters 22: 1859-1869; and Matthes et al. (1984) EMBO J. 3: 801-805. According to such methods, oligonucleotides are synthesized, purified, annealed to their complementary strand, ligated and then optionally cloned into suitable vectors. And if so desired, the polynucleotides and polypeptides of the invention can be custom ordered from any of a number of commercial suppliers.

Homologous Sequences. Sequences homologous to those provided in the Sequence Listing derived from Arabidopsis thaliana or from other plants of choice, are also an aspect of the invention. Homologous sequences can be derived from any plant including monocots and dicots and in particular agriculturally important plant species, including but not limited to, crops such as soybean, wheat, corn (maize), potato, cotton, rice, rape, oilseed rape (including canola), sunflower, alfalfa, clover, sugarcane, and turf; or fruits and vegetables, such as banana, blackberry, blueberry, strawberry, and raspberry, cantaloupe, carrot, cauliflower, coffee, cucumber, eggplant, grapes, honeydew, lettuce, mango, melon, onion, papaya, peas, peppers, pineapple, pumpkin, spinach, squash, sweet corn, tobacco, tomato, tomatillo, watermelon, rosaceous fruits (such as apple, peach, pear, cherry and plum) and vegetable brassicas (such as broccoli, cabbage, cauliflower, Brussels sprouts, and kohlrabi). Other crops, including fruits and vegetables, whose phenotype can be changed and which comprise homologous sequences include barley; rye; millet; sorghum; currant; avocado; citrus fruits such as oranges, lemons, grapefruit and tangerines, artichoke, cherries; nuts such as the walnut and peanut; endive; leek; roots such as arrowroot, beet, cassava, turnip, radish, yam, and sweet potato; and beans. The homologous sequences may also be derived from woody species, such as pine, poplar and eucalyptus, or mint or other labiates. In addition, homologous sequences may be derived from plants that are evolutionarily-related to crop plants, but which may not have yet been used as crop plants. Examples include deadly nightshade (Atropa belladona), related to tomato; jimson weed (Datura strommium), related to peyote; and teosinte (Zea species), related to corn (maize).

Ortholops and Paralogs. Homologous sequences as described above can comprise orthologous or paralogous sequences. Several different methods are known by those of skill in the art for identifying and defining these functionally homologous sequences. Three general methods for defining orthologs and paralogs are described; an ortholog or paralog, including equivalogs, may be identified by one or more of the methods described below.

Within a single plant species, gene duplication may cause two copies of a particular gene, giving rise to two or more genes with similar sequence and often similar function known as paralogs. A paralog is therefore a similar gene formed by duplication within the same species. Paralogs typically cluster together or in the same clade (a group of similar genes) when a gene family phylogeny is analyzed using programs such as CLUSTAL (Thompson et al. (1994) Nucleic Acids Res. 22: 4673-4680; Higgins et al. (1996) Methods Enzymol. 266: 383-402). Groups of similar genes can also be identified with pair-wise BLAST analysis (Feng and Doolittle (1987) J. Mol. Evol. 25: 351-360). For example, a clade of very similar MADS domain transcription factors from Arabidopsis all share a common function in flowering time (Ratcliffe et al. (2001) Plant Physiol. 126: 122-132), and a group of very similar AP2 domain transcription factors from Arabidopsis are involved in tolerance of plants to freezing (Gilmour et al. (1998) Plant J. 16: 433-442). Analysis of groups of similar genes with similar function that fall within one clade can yield sub-sequences that are particular to the clade. These sub-sequences, known as consensus sequences, can not only be used to define the sequences within each clade, but define the functions of these genes; genes within a clade may contain paralogous sequences, or orthologous sequences that share the same function (for example, Mount (2001), in Bioinformatics: Sequence and Genome Analysis, Cold Spring Harbor Laboratory Press, Cold Spring Harbor, N.Y., page 543).

Speciation, the production of new species from a parental species, can also give rise to two or more genes with similar sequence and similar function. These genes, termed orthologs, often have an identical function within their host plants and are often interchangeable between species without losing function. Because plants have common ancestors, many genes in any plant species will have a corresponding orthologous gene in another plant species. Once a phylogenic tree for a gene family of one species has been constructed using a program such as CLUSTAL (Thompson et al. (1994) Nucleic Acids Res. 22: 4673-4680; Higgins et al. (1996) supra) potential orthologous sequences can be placed into the phylogenetic tree and their relationship to genes from the species of interest can be determined. Orthologous sequences can also be identified by a reciprocal BLAST strategy. Once an orthologous sequence has been identified, the function of the ortholog can be deduced from the identified function of the reference sequence.

Transcription factor gene sequences are conserved across diverse eukaryotic species lines (Goodrich et al. (1993) Cell 75: 519-530; Lin et al. (1991) Nature 353: 569-571; Sadowski et al. (1988) Nature 335: 563-564). Plants are no exception to this observation; diverse plant species possess transcription factors that have similar sequences and functions.

Orthologous genes from different organisms have highly conserved functions, and very often essentially identical functions (Lee et al. (2002) Genome Res. 12: 493-502; Remm et al. (2001) J. Mol. Biol. 314: 1041-1052). Paralogous genes, which have diverged through gene duplication, may retain similar functions of the encoded proteins. In such cases, paralogs can be used interchangeably with respect to certain embodiments of the instant invention (for example, transgenic expression of a coding sequence). An example of such highly related paralogs is the CBF family, with three well-defined members in Arabidopsis and at least one ortholog in Brassica napus (United States Patent Application 20040098764), all of which control pathways involved in both freezing and drought stress (Gilmour et al. (1998) Plant J. 16: 433-442; Jaglo et al. (1998) Plant Physiol. 127: 910-917).

The following references represent a small sampling of the many studies that demonstrate that conserved transcription factor genes from diverse species are likely to function similarly (i.e., regulate similar target sequences and control the same traits), and that transcription factors may be transformed into diverse species to confer or improve traits.
(1) Distinct Arabidopsis transcription factors, including G28 (SEQ ID NO: 48, U.S. Pat. No. 6,664,446), G482 (US Patent Application 20040045049), G867 (US Patent Application 20040098764), and G1073 (U.S. Pat. No. 6,717,034), have been shown to confer abiotic stress tolerance when the sequences are overexpressed. The polypeptides sequences belong to distinct clades of transcription factor polypeptides that include members from diverse species. In each case, a significant number of sequences derived from both dicots and monocots have been shown to confer tolerance to various abiotic stresses when the sequences were overexpressed (unpublished data).
(2) The Arabidopsis NPR1 gene regulates systemic acquired resistance (SAR); over-expression of NPR1 leads to enhanced resistance in Arabidopsis. When either Arabidopsis NPR1 or the rice NPR1 ortholog was overexpressed in rice (which, as a monocot, is diverse from Arabidopsis), challenge with the rice bacterial blight pathogen Xanthomonas oryzae pv. Oryzae, the transgenic plants displayed enhanced resistance (Chem et al. (2001) Plant J. 27: 101-113). NPR1 acts through activation of expression of transcription factor genes, such as TGA2 (Fan and Dong (2002) Plant Cell 14: 1377-1389).
(3) E2F genes are involved in transcription of plant genes for proliferating cell nuclear antigen (PCNA). Plant E2Fs share a high degree of similarity in amino acid sequence between monocots and dicots, and are even similar to the conserved domains of the animal E2Fs. Such conservation indicates a functional similarity between plant and animal E2Fs. E2F transcription factors that regulate meristem development act through common cis-elements, and regulate related (PCNA) genes (Kosugi and Ohashi (2002) Plant J. 29: 45-59).
(4) The ABI5 gene (abscisic acid (ABA) insensitive 5) encodes a basic leucine zipper factor required for ABA response in the seed and vegetative tissues. Co-transformation experiments with ABI5 cDNA constructs in rice protoplasts resulted in specific transactivation of the ABA-inducible wheat, Arabidopsis, bean, and barley promoters. These results demonstrate that sequentially similar ABI5 transcription factors are key targets of a conserved ABA signaling pathway in diverse plants (Gampala et al. (2001) J. Biol. Chem. 277: 1689-1694).
(5) Sequences of three Arabidopsis GAMYB-like genes were obtained on the basis of sequence similarity to GAMYB genes from barley, rice, and L. temulentum. These three Arabidopsis genes were determined to encode transcription factors (AtMYB33, AtMYB65, and AtMYB 101) and could substitute for a barley GAMYB and control alpha-amylase expression (Gocal et al. (2001) Plant Physiol. 127: 1682-1693).
(6) The floral control gene LEAFY from Arabidopsis can dramatically accelerate flowering in numerous dicotyledonous plants. Constitutive expression of Arabidopsis LEAFY also caused early flowering in transgenic rice (a monocot), with a heading date that was 26-34 days earlier than that of wild-type plants. These observations indicate that floral regulatory genes from Arabidopsis are useful tools for heading date improvement in cereal crops (He et al. (2000) Transgenic Res. 9: 223-227).
(7) Bioactive gibberellins (GAs) are essential endogenous regulators of plant growth. GA signaling tends to be conserved across the plant kingdom. GA signaling is mediated via GAI, a nuclear member of the GRAS family of plant transcription factors. Arabidopsis GAI has been shown to function in rice to inhibit gibberellin response pathways (Fu et al. (2001) Plant Cell 13: 1791-1802).
(8) The Arabidopsis gene SUPERMAN (SUP), encodes a putative transcription factor that maintains the boundary between stamens and carpels. By over-expressing Arabidopsis SUP in rice, the effect of the gene's presence on whorl boundaries was shown to be conserved. This demonstrated that SUP is a conserved regulator of floral whorl boundaries and affects cell proliferation (Nandi et al. (2000) Curr. Biol. 10: 215-218).
(9) Maize, petunia and Arabidopsis myb transcription factors that regulate flavonoid biosynthesis are genetically similar and affect the same trait in their native species. Therefore, sequence and function of these myb transcription factors correlate with each other in these diverse species (Borevitz et al. (2000) Plant Cell 12: 2383-2394).
(10) Wheat reduced height-1 (Rht-B1/Rht-D1) and maize dwarf-8 (d8) genes are orthologs of the Arabidopsis gibberellin insensitive (GAI) gene. Both of these genes have been used to produce dwarf grain varieties that have improved grain yield. These genes encode proteins that resemble nuclear transcription factors and contain an SH2-like domain, indicating that phosphotyrosine may participate in gibberellin signaling.

Transgenic rice plants containing a mutant GAI allele from Arabidopsis have been shown to produce reduced responses to gibberellin and are dwarfed, indicating that mutant GAI orthologs could be used to increase yield in a wide range of crop species (Peng et al. (1999) Nature 400: 256-261).

Transcription factors that are homologous to the listed AP2 transcription factors will typically share at least about 69% and 62% amino acid sequence identity in their AP2 and EDLL domains, respectively, as seen by the examples shown to confer low nitrogen or abiotic stress tolerance in Table 1. Transcription factors that are homologous to the listed sequences should share at least 40% amino acid sequence identity over the entire length of the polypeptide.

At the nucleotide level, the sequences of the invention will typically share at least about 40% or greater nucleotide sequence identity to one or more of the listed full-length sequences, or to a listed sequence but excluding or outside of the region(s) encoding a known consensus sequence or consensus DNA-binding site, or outside of the region(s) encoding one or all conserved domains. The degeneracy of the genetic code enables major variations in the nucleotide sequence of a polynucleotide while maintaining the amino acid sequence of the encoded protein.

Percent identity can be determined electronically, e.g., by using the MEGALIGN program (DNASTAR, Inc. Madison, Wis.). The MEGALIGN program can create alignments between two or more sequences according to different methods, for example, the clustal method (for example, Higgins and Sharp (1988) Gene 73: 237-244). The clustal algorithm groups sequences into clusters by examining the distances between all pairs. The clusters are aligned pairwise and then in groups. Other alignment algorithms or programs may be used, including FASTA, BLAST, or ENTREZ, FASTA and BLAST, and which may be used to calculate percent similarity. These are available as a part of the GCG sequence analysis package (University of Wisconsin, Madison, Wis.), and can be used with or without default settings. ENTREZ is available through the National Center for Biotechnology Information. In one embodiment, the percent identity of two sequences can be determined by the GCG program with a gap weight of 1, e.g., each amino acid gap is weighted as if it were a single amino acid or nucleotide mismatch between the two sequences (U.S. Pat. No. 6,262,333).

Other techniques for alignment are described in Methods in Enzymology, vol. 266, Computer Methods for Macromolecular Sequence Analysis (1996), ed. Doolittle, Academic Press, Inc., San Diego, Calif., USA. Preferably, an alignment program that permits gaps in the sequence is utilized to align the sequences. The Smith-Waterman is one type of algorithm that permits gaps in sequence alignments (Shpaer (1997) Methods Mol. Biol. 70: 173-187). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

The percentage similarity between two polypeptide sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no similarity between the two amino acid sequences are not included in determining percentage similarity. Percent identity between polynucleotide sequences can also be counted or calculated by other methods known in the art, e.g., the Jotun Hein method (for example, Hein (1990) Methods Enzymol. 183: 626-645). Identity between sequences can also be determined by other methods known in the art, e.g., by varying hybridization conditions (US Patent Application No. 20010010913).

Thus, the invention provides methods for identifying a sequence similar or paralogous or orthologous or homologous to one or more polynucleotides as noted herein, or one or more target polypeptides encoded by the polynucleotides, or otherwise noted herein and may include linking or associating a given plant phenotype or gene function with a sequence. In the methods, a sequence database is provided (locally or across an internet or intranet) and a query is made against the sequence database using the relevant sequences herein and associated plant phenotypes or gene functions.

In addition, one or more polynucleotide sequences or one or more polypeptides encoded by the polynucleotide sequences may be used to search against a BLOCKS (Bairoch et al. (1997) Nucleic Acids Res. 25: 217-221), PFAM, and other databases which contain previously identified and annotated motifs, sequences and gene functions. Methods that search for primary sequence patterns with secondary structure gap penalties (Smith et al. (1992) Protein Engineering 5: 35-51) as well as algorithms such as Basic Local Alignment Search Tool (BLAST; Altschul (1993) J. Mol. Evol. 36: 290-300; Altschul et al. (1990) J. Mol. Biol. 215: 403-410), BLOCKS (Henikoff and Henikoff (1991) Nucleic Acids Res. 19: 6565-6572), Hidden Markov Models (HMM; Eddy (1996) Curr. Opin. Sir. Biol. 6: 361-365; Sonnhammer et al. (1997) Proteins 28: 405-420), and the like, can be used to manipulate and analyze polynucleotide and polypeptide sequences encoded by polynucleotides. These databases, algorithms and other methods are well known in the art and are described in Ausubel et al. (1997) Short Protocols in Molecular Biology, John Wiley & Sons, New York, N.Y., unit 7.7; and in Meyers (1995) Molecular Biology and Biotechnology, Wiley VCH, New York, N.Y., p 856-853.

A further method for identifying or confirming that specific homologous sequences control the same function is by comparison of the transcript profile(s) obtained upon overexpression or knockout of two or more related transcription factors. Since transcript profiles are diagnostic for specific cellular states, one skilled in the art will appreciate that genes that have a highly similar transcript profile (e.g., with greater than 50% regulated transcripts in common, more preferably with greater than 70% regulated transcripts in common, most preferably with greater than 90% regulated transcripts in common) will have highly similar functions. Fowler et al. (2002) Plant Cell, 14: 1675-1679, have shown that three paralogous AP2 family genes (CBF1, CBF2 and CBF3), each of which is induced upon cold treatment, and each of which can condition improved freezing tolerance, have highly similar transcript profiles. Once a transcription factor has been shown to provide a specific function, its transcript profile becomes a diagnostic tool to determine whether putative paralogs or orthologs have the same function.

Furthermore, methods using manual alignment of sequences similar or homologous to one or more polynucleotide sequences or one or more polypeptides encoded by the polynucleotide sequences may be used to identify regions of similarity and AP2 domains. Such manual methods are well-known of those of skill in the art and can include, for example, comparisons of tertiary structure between a polypeptide sequence encoded by a polynucleotide with a known function, and a polypeptide sequence encoded by a polynucleotide sequence for which a function has not yet been determined. Such examples of tertiary structure may comprise predicted α-helices, β-sheets, amphipathic helices, leucine zipper motifs, zinc finger motifs, proline-rich regions, cysteine repeat motifs, and the like.

Orthologs and paralogs of presently disclosed transcription factors may be cloned using compositions provided by the present invention according to methods well known in the art. cDNAs can be cloned using mRNA from a plant cell or tissue that expresses one of the present transcription factors. Appropriate mRNA sources may be identified by interrogating Northern blots with probes designed from the present transcription factor sequences, after which a library is prepared from the mRNA obtained from a positive cell or tissue. Transcription factor-encoding cDNA is then isolated using, for example, PCR, using primers designed from a presently disclosed transcription factor gene sequence, or by probing with a partial or complete cDNA or with one or more sets of degenerate probes based on the disclosed sequences. The cDNA library may be used to transform plant cells. Expression of the cDNAs of interest is detected using, for example, methods disclosed herein such as microarrays, Northern blots, quantitative PCR, or any other technique for monitoring changes in expression. Genomic clones may be isolated using similar techniques to those.

Examples of orthologs of the Arabidopsis polypeptide sequences SEQ ID NOs: 2, 4, 6, and 8 include SEQ ID NOs: 10, 12, 14, 16, 18, 20, 22, 24, 26, 28, 30, 32, 34 and 36, and other functionally similar orthologs that may be discovered using the methods found in Examples X and XI. In addition to the sequences in the Sequence Listing, the invention encompasses isolated nucleotide sequences that are sequentially and structurally similar to Arabidopsis sequences G30, G1791, and G1792, soybean G3518 and G3520, rice G3380, G3381, G3383, G3515, and G3737, and corn G3516 and G3517 (SEQ ID NO: 7, 3, 1, 21, 25, 9, 11, 13, 15, 31, 17, and 19, respectively) and function in a plant by increasing low nitrogen and/or abiotic stress tolerance, particularly when overexpressed. These polypeptide sequences represent clade members that function similarly to G1792 by conferring low nitrogen and other abiotic stress tolerance, and show significant sequence similarity to G1792, as shown by their respective identities to the AP2 and EDLL domains of G1792, as shown in Table 1.

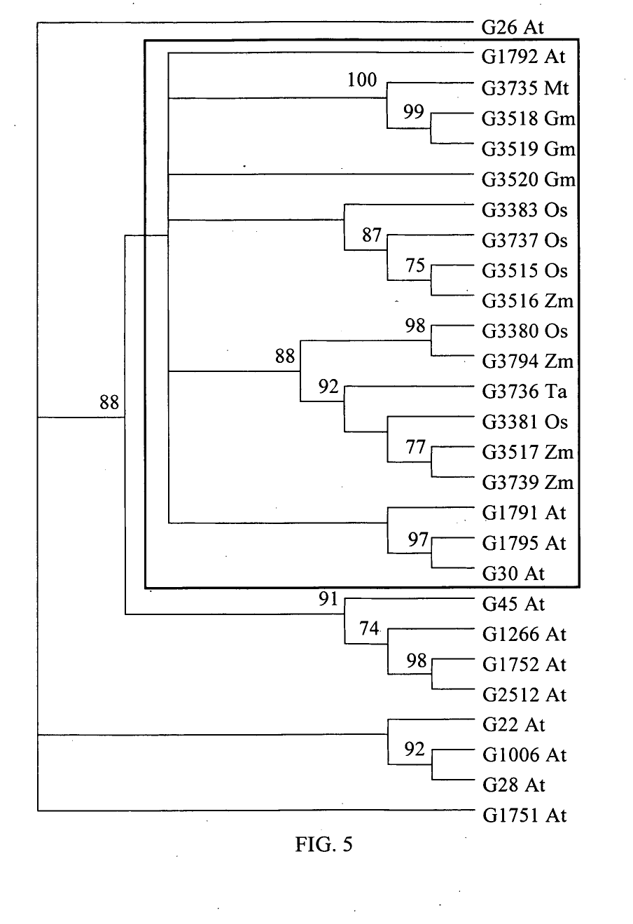

Since a number of these polynucleotide sequences in the G1792 clade of transcription factor polypeptides are phylogenetically related (FIG. 5), similar in sequence, are derived from diverse plant species, and have been shown to increase a plant's low nitrogen and/or abiotic stress tolerance, one skilled in the art would predict that other similar, phylogenetically related sequences would also increase a plant's tolerance to abiotic and/or low nitrogen stresses.

Identifying Polynucleotides or Nucleic Acids by Hybridization. Polynucleotides homologous to the sequences illustrated in the Sequence Listing and tables can be identified, e.g., by hybridization to each other under stringent or under highly stringent conditions. Single stranded polynucleotides hybridize when they associate based on a variety of well characterized physical-chemical forces, such as hydrogen bonding, solvent exclusion, base stacking and the like. The stringency of a hybridization reflects the degree of sequence identity of the nucleic acids involved, such that the higher the stringency, the more similar are the two polynucleotide strands. Stringency is influenced by a variety of factors, including temperature, salt concentration and composition, organic and non-organic additives, solvents, etc. present in both the hybridization and wash solutions and incubations (and number thereof), as described in more detail in the references cited above.

Encompassed by the invention are polynucleotide sequences that are capable of hybridizing to the claimed polynucleotide sequences, including any of the transcription factor polynucleotides within the Sequence Listing, and fragments thereof under various conditions of stringency (for example, Wahl and Berger, in Berger and Kimmel (1987) supra, pages 399-407, and Kimmel, in and Berger and Kimmel (1987) supra, pages 507-511). In addition to the nucleotide sequences listed in the Sequence Listing, full length cDNA, orthologs, and paralogs of the present nucleotide sequences may be identified and isolated using well-known methods. The cDNA libraries, orthologs, and paralogs of the present nucleotide sequences may be screened using hybridization methods to determine their utility as hybridization target or amplification probes.

With regard to hybridization, conditions that are highly stringent, and means for achieving them, are well known in the art (for example, in Sambrook et al. (1989) supra; Berger and Kimmel (1987) supra, pages 467-469; and Anderson and Young (1985) “Quantitative Filter Hybridisation.” In: Hames and Higgins, ed., Nucleic Acid Hybridisation, A Practical Approach, Oxford, IRL Press, 73-111.

Stability of DNA duplexes is affected by such factors as base composition, length, and degree of base pair mismatch. Hybridization conditions may be adjusted to allow DNAs of different sequence relatedness to hybridize. The melting temperature (Tₘ) is defined as the temperature when 50% of the duplex molecules have dissociated into their constituent single strands. The melting temperature of a perfectly matched duplex, where the hybridization buffer contains formamide as a denaturing agent, may be estimated by the following equations:

(I) DNA-DNA:
Tₘ(° C.)=81.5+16.6(log [Na+])+0.41(% G+C)−0.62(% formamide)−500/L

(II) DNA-RNA:
Tₘ(° C.)=79.8+18.5(log [Na+])+0.58(% G+C)+0.12(% G+C)²−0.5(% formamide)−820/L

(III) RNA-RNA:
Tₘ(° C.)=79.8+18.5(log [Na+])+0.58(% G+C)+0.12(% G+C)²−0.35(% formamide)−820/L

where L is the length of the duplex formed, [Na+] is the molar concentration of the sodium ion in the hybridization or washing solution, and % G+C is the percentage of (guanine+cytosine) bases in the hybrid. For imperfectly matched hybrids, approximately 1° C. is required to reduce the melting temperature for each 1% mismatch.

Hybridization experiments are generally conducted in a buffer of pH between 6.8 to 7.4, although the rate of hybridization is nearly independent of pH at ionic strengths likely to be used in the hybridization buffer (Anderson et al. (1985) supra). In addition, one or more of the following may be used to reduce non-specific hybridization: sonicated salmon sperm DNA or another non-complementary DNA, bovine serum albumin, sodium pyrophosphate, sodium dodecyl sulfate (SDS), polyvinyl-pyrrolidone, ficoll and Denhardt's solution. Dextran sulfate and polyethylene glycol 6000 act to exclude DNA from solution, thus raising the effective probe DNA concentration and the hybridization signal within a given unit of time. In some instances, conditions of even greater stringency may be desirable or required to reduce non-specific and/or background hybridization. These conditions may be created with the use of higher temperature, lower ionic strength and higher concentration of a denaturing agent such as formamide.

Stringency conditions can be adjusted to screen for moderately similar fragments such as homologous sequences from distantly related organisms, or to highly similar fragments such as genes that duplicate functional enzymes from closely related organisms. The stringency can be adjusted either during the hybridization step or in the post-hybridization washes. Salt concentration, formamide concentration, hybridization temperature and probe lengths are variables that can be used to alter stringency (as described by the formula above). As a general guidelines high stringency is typically performed at Tₘ−5° C. to Tₘ−20° C., moderate stringency at Tₘ−20° C. to Tₘ−35° C. and low stringency at Tₘ−35° C. to Tₘ−50° C. for duplex>150 base pairs. Hybridization may be performed at low to moderate stringency (25-50° C. below Tₘ), followed by post-hybridization washes at increasing stringencies. Maximum rates of hybridization in solution are determined empirically to occur at Tₘ−25° C. for DNA-DNA duplex and Tₘ−15° C. for RNA-DNA duplex. Optionally, the degree of dissociation may be assessed after each wash step to determine the need for subsequent, higher stringency wash steps.

High stringency conditions may be used to select for nucleic acid sequences with high degrees of identity to the disclosed sequences. An example of stringent hybridization conditions obtained in a filter-based method such as a Southern or northern blot for hybridization of complementary nucleic acids that have more than 100 complementary residues is about 5° C. to 20° C. lower than the thermal melting point (Tₘ) for the specific sequence at a defined ionic strength and pH. Conditions used for hybridization may include about 0.02 M to about 0.15 M sodium chloride, about 0.5% to about 5% casein, about 0.02% SDS or about 0.1% N-laurylsarcosine, about 0.001 M to about 0.03 M sodium citrate, at hybridization temperatures between about 50° C. and about 70° C. More preferably, high stringency conditions are about 0.02 M sodium chloride, about 0.5% casein, about 0.02% SDS, about 0.001 M sodium citrate, at a temperature of about 50° C. Nucleic acid molecules that hybridize under stringent conditions will typically hybridize to a probe based on either the entire DNA molecule or selected portions, e.g., to a unique subsequence, of the DNA.

Stringent salt concentration will ordinarily be less than about 750 mM NaCl and 75 mM trisodium citrate. Increasingly stringent conditions may be obtained with less than about 500 mM NaCl and 50 mM trisodium citrate, to even greater stringency with less than about 250 mM NaCl and 25 mM trisodium citrate. Low stringency hybridization can be obtained in the absence of organic solvent, e.g., formamide, whereas high stringency hybridization may be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. with formamide present. Varying additional parameters, such as hybridization time, the concentration of detergent, e.g., sodium dodecyl sulfate (SDS) and ionic strength, are well known to those skilled in the art. Various levels of stringency are accomplished by combining these various conditions as needed.

The washing steps that follow hybridization may also vary in stringency; the post-hybridization wash steps primarily determine hybridization specificity, with the most critical factors being temperature and the ionic strength of the final wash solution. Wash stringency can be increased by decreasing salt concentration or by increasing temperature. Stringent salt concentration for the wash steps will preferably be less than about 30 mM NaCl and 3 mM trisodium citrate, and most preferably less than about 15 mM NaCl and 1.5 mM trisodium citrate.

Thus, hybridization and wash conditions that may be used to bind and remove polynucleotides with less than the desired homology to the nucleic acid sequences or their complements that encode the present transcription factors include, for example:

6×SSC at 65° C.;

50% formamide, 4×SSC at 42° C.; or

0.5×SSC, 0.1% SDS at 65° C.;

with, for example, two wash steps of 10-30 minutes each. Useful variations on these conditions will be readily apparent to those skilled in the art.

A person of skill in the art would not expect substantial variation among polynucleotide species encompassed within the scope of the present invention because the highly stringent conditions set forth in the above formulae yield structurally similar polynucleotides.

If desired, one may employ wash steps of even greater stringency, including about 0.2×SSC, 0.1% SDS at 65° C. and washing twice, each wash step being about 30 minutes, or about 0.1×SSC, 0.1% SDS at 65° C. and washing twice for 30 minutes. The temperature for the wash solutions will ordinarily be at least about 25° C., and for greater stringency at least about 42° C. Hybridization stringency may be increased further by using the same conditions as in the hybridization steps, with the wash temperature raised about 3° C. to about 5° C., and stringency may be increased even further by using the same conditions except the wash temperature is raised about 6° C. to about 9° C. For identification of less closely related homologs, wash steps may be performed at a lower temperature, e.g., 50° C.

An example of a low stringency wash step employs a solution and conditions of at least 25° C. in 30 mM NaCl, 3 mM trisodium citrate, and 0.1% SDS over 30 minutes. Greater stringency may be obtained at 42° C. in 15 mM NaCl, with 1.5 mM trisodium citrate, and 0.1% SDS over 30 minutes. Even higher stringency wash conditions are obtained at 65° C.-68° C. in a solution of 15 mM NaCl, 1.5 mM trisodium citrate, and 0.1% SDS. Wash procedures will generally employ at least two final wash steps. Additional variations on these conditions will be readily apparent to those skilled in the art (for example, US Patent Application No. 20010010913).

Stringency conditions can be selected such that an oligonucleotide that is perfectly complementary to the coding oligonucleotide hybridizes to the coding oligonucleotide with at least about a 5-10× higher signal to noise ratio than the ratio for hybridization of the perfectly complementary oligonucleotide to a nucleic acid encoding a transcription factor known as of the filing date of the application. It may be desirable to select conditions for a particular assay such that a higher signal to noise ratio, that is, about 15× or more, is obtained. Accordingly, a subject nucleic acid will hybridize to a unique coding oligonucleotide with at least a 2× or greater signal to noise ratio as compared to hybridization of the coding oligonucleotide to a nucleic acid encoding known polypeptide. The particular signal will depend on the label used in the relevant assay, e.g., a fluorescent label, a calorimetric label, a radioactive label, or the like. Labeled hybridization or PCR probes for detecting related polynucleotide sequences may be produced by oligolabeling, nick translation, end-labeling, or PCR amplification using a labeled nucleotide.

Identifying Polynucleotides or Nucleic Acids with Expression Libraries. In addition to hybridization methods, transcription factor homolog polypeptides can be obtained by screening an expression library using antibodies specific for one or more transcription factors. With the provision herein of the disclosed transcription factor, and transcription factor homolog nucleic acid sequences, the encoded polypeptide(s) can be expressed and purified in a heterologous expression system (for example, E. coli) and used to raise antibodies (monoclonal or polyclonal) specific for the polypeptide(s) in question. Antibodies can also be raised against synthetic peptides derived from the amino acid sequences or subsequences of a transcription factor or transcription factor homolog. Methods of raising antibodies are well known in the art and are described in Harlow and Lane (1988), Antibodies: A Laboratory Manual, Cold Spring Harbor Laboratory, New York. Such antibodies can then be used to screen an expression library produced from the plant from which it is desired to clone additional transcription factor homologs, using the methods described above. The selected cDNAs can be confirmed by sequencing and enzymatic activity.

Sequence Variations. It will readily be appreciated by those of skill in the art, that any of a variety of polynucleotide sequences are capable of encoding the transcription factors and transcription factor homolog polypeptides of the invention. Due to the degeneracy of the genetic code, many different polynucleotides can encode identical and/or substantially similar polypeptides in addition to those sequences illustrated in the Sequence Listing. Nucleic acids having a sequence that differs from the sequences shown in the Sequence Listing, or complementary sequences, that encode functionally equivalent peptides (i.e., peptides having some degree of equivalent or similar biological activity) but differ in sequence from the sequence shown in the Sequence Listing due to degeneracy in the genetic code, are also within the scope of the invention.

Altered polynucleotide sequences encoding polypeptides include those sequences with deletions; insertions, or substitutions of different nucleotides, resulting in a polynucleotide encoding a polypeptide with at least one functional characteristic of the instant polypeptides. Included within this definition are polymorphisms which may or may not be readily detectable using a particular oligonucleotide probe of the polynucleotide encoding the instant polypeptides, and improper or unexpected hybridization to allelic variants, with a locus other than the normal chromosomal locus for the polynucleotide sequence encoding the instant polypeptides.

Allelic variant refers to any of two or more alternative forms of a gene occupying the same chromosomal locus. Allelic variation arises naturally through mutation, and may result in phenotypic polymorphism within populations. Gene mutations can be silent (i.e., no change in the encoded polypeptide) or may encode polypeptides having altered amino acid sequence. The term allelic variant is also used herein to denote a protein encoded by an allelic variant of a gene. Splice variant refers to alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a protein encoded by a splice variant of an mRNA transcribed from a gene.

Those skilled in the art would recognize that, for example, G1792, SEQ ID NO: 2, represents a single transcription factor; allelic variation and alternative splicing may be expected to occur. Allelic variants of SEQ ID NO: 1 can be cloned by probing cDNA or genomic libraries from different individual organisms according to standard procedures. Allelic variants of the DNA sequence shown in SEQ ID NO: 1, including those containing silent mutations and those in which mutations result in amino acid sequence changes, are within the scope of the present invention, as are proteins which are allelic variants of SEQ ID NO: 2. cDNAs generated from alternatively spliced mRNAs, which retain the properties of the transcription factor are included within the scope of the present invention, as are polypeptides encoded by such cDNAs and mRNAs. Allelic variants and splice variants of these sequences can be cloned by probing cDNA or genomic libraries from different individual organisms or tissues according to standard procedures known in the art (U.S. Pat. No. 6,388,064).

Thus, in addition to the sequences set forth in the Sequence Listing, the invention also encompasses related nucleic acid molecules that include allelic or splice variants, and sequences that are complementary. Related nucleic acid molecules also include nucleotide sequences encoding a polypeptide comprising a substitution, modification, addition and/or deletion of one or more amino acid residues. Such related polypeptides may comprise, for example, additions and/or deletions of one or more N-linked or O-linked glycosylation sites, or an addition and/or a deletion of one or more cysteine residues.

For example, Table 2 illustrates, for example, that the codons AGC, AGT, TCA, TCC, TCG, and TCT all encode the same amino acid: serine. Accordingly, at each position in the sequence where there is a codon encoding serine, any of the above trinucleotide sequences can be used without altering the encoded polypeptide.

TABLE 2

| | |
|---|---|
| Amino acid | Possible Codons |

| | | | |
|---|---|---|---|
| Alanine | Ala | A | GCA GCC GCG GCU |
| Cysteine | Cys | C | TGC TGT |
| Aspartic acid | Asp | D | GAC GAT |
| Glutamic acid | Glu | E | GAA GAG |
| Phenylalanine | Phe | F | TTC TTT |
| Glycine | Gly | G | GGA GGC GGG GGT |
| Histidine | His | H | CAC CAT |
| Isoleucine | Ile | I | ATA ATC ATT |
| Lysine | Lys | K | AAA AAG |
| Leucine | Leu | L | TTA TTG CTA CTC CTG CTT |
| Methionine | Met | M | ATG |
| Asparagine | Asn | N | AAC AAT |
| Proline | Pro | P | CCA CCC CCG CCT |
| Glutamine | Gln | Q | CAA CAG |
| Arginine | Arg | R | AGA AGG CGA CGC CGG CGT |
| Serine | Ser | S | AGC AGT TCA TCC TCG TCT |
| Threonine | Thr | T | ACA ACC ACG ACT |
| Valine | Val | V | GTA GTC GTG GTT |
| Tryptophan | Trp | W | TGG |
| Tyrosine | Tyr | Y | TAC TAT |

Sequence alterations that do not change the amino acid sequence encoded by the polynucleotide are termed “silent” variations. With the exception of the codons ATG and TGG, encoding methionine and tryptophan, respectively, any of the possible codons for the same amino acid can be substituted by a variety of techniques, e.g., site-directed mutagenesis, available in the art. Accordingly, any and all such variations of a sequence selected from the above table are a feature of the invention.

In addition to silent variations, other conservative variations that alter one, or a few amino acids in the encoded polypeptide, can be made without altering the function of the polypeptide, these conservative variants are, likewise, a feature of the invention.

For example, substitutions, deletions and insertions introduced into the sequences provided in the Sequence Listing, are also envisioned by the invention. Such sequence modifications can be engineered into a sequence by site-directed mutagenesis (Wu, editor; Methods Enzymol. (1993) vol. 217, Academic Press) or the other methods noted below. Amino acid substitutions are typically of single residues; insertions usually will be on the order of about from 1 to 10 amino acid residues; and deletions will range about from 1 to 30 residues. In one embodiment, deletions or insertions are made in adjacent pairs, e.g., a deletion of two residues or insertion of two residues. Substitutions, deletions, insertions or any combination thereof can be combined to arrive at a sequence. The mutations that are made in the polynucleotide encoding the transcription factor should not place the sequence out of reading frame and should not create complementary regions that could produce secondary mRNA structure. Preferably, the polypeptide encoded by the DNA performs the desired function.

Conservative substitutions are those in which at least one residue in the amino acid sequence has been removed and a different residue inserted in its place. Such substitutions generally are made in accordance with the Table 3 when it is desired to maintain the activity of the protein. Table 3 shows amino acids which can be substituted for an amino acid in a protein and which are typically regarded as conservative substitutions. In one embodiment, transcriptions factors listed in the Sequence Listing may have up to 10 conservative substitutions and retain their function. In another embodiment, transcription factors listed in the Sequence Listing may have more than 10 conservative substitutions and still retain their function.

TABLE 3

| | Conservative |
|---|---|
| Residue | Substitutions |
| Ala | Ser |
| Arg | Lys |
| Asn | Gln; His |
| Asp | Glu |
| Gln | Asn |
| Cys | Ser |
| Glu | Asp |
| Gly | Pro |
| His | Asn; Gln |
| Ile | Leu, Val |
| Leu | Ile; Val |
| Lys | Arg; Gln |
| Met | Leu; Ile |
| Phe | Met; Leu; Tyr |
| Ser | Thr; Gly |
| Thr | Ser; Val |
| Trp | Tyr |
| Tyr | Trp; Phe |
| Val | Ile; Leu |

Similar substitutions are those in which at least one residue in the amino acid sequence has been removed and a different residue inserted in its place. Such substitutions generally are made in accordance with the Table 4 when it is desired to maintain the activity of the protein. Table 4 shows amino acids which can be substituted for an amino acid in a protein and which are typically regarded as structural and functional substitutions. For example, a residue in column 1 of Table 4 may be substituted with a residue in column 2; in addition, a residue in column 2 of Table 4 may be substituted with the residue of column 1.

TABLE 4

| Residue | Similar Substitutions |
|---|---|
| Ala | Ser; Thr; Gly; Val; Leu; Ile |
| Arg | Lys; His; Gly |
| Asn | Gln; His; Gly; Ser; Thr |
| Asp | Glu, Ser; Thr |
| Gln | Asn; Ala |
| Cys | Ser; Gly |
| Glu | Asp |
| Gly | Pro; Arg |
| His | Asn; Gln; Tyr; Phe; Lys; Arg |
| Ile | Ala; Leu; Val; Gly; Met |
| Leu | Ala; Ile; Val; Gly; Met |
| Lys | Arg; His; Gln; Gly; Pro |
| Met | Leu; Ile; Phe |
| Phe | Met; Leu; Tyr; Trp; His; Val; Ala |
| Ser | Thr; Gly; Asp; Ala; Val; Ile; His |
| Thr | Ser; Val; Ala; Gly |
| Trp | Tyr; Phe; His |
| Tyr | Trp; Phe; His |
| Val | Ala; Ile; Leu; Gly; Thr; Ser; Glu |

Substitutions that are less conservative than those in Table 4 can be selected by picking residues that differ more significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. The substitutions which in general are expected to produce the greatest changes in protein properties will be those in which (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine.

Expression and Modification of Polypeptides. Typically, polynucleotide sequences of the invention are incorporated into recombinant DNA (or RNA) molecules that direct expression of polypeptides of the invention in appropriate host cells, transgenic plants, in vitro translation systems, or the like. Due to the inherent degeneracy of the genetic code, nucleic acid sequences which encode substantially the same or a functionally equivalent amino acid sequence can be substituted for any listed sequence to provide for cloning and expressing the relevant homolog.

The transgenic plants of the present invention comprising recombinant polynucleotide sequences are generally derived from parental plants, which may themselves be non-transformed (or non-transgenic) plants. These transgenic plants may either have a transcription factor gene “knocked out” (for example, with a genomic insertion by homologous recombination, an antisense or ribozyme construct) or expressed to a normal or wild-type extent. However, overexpressing transgenic “progeny” plants will exhibit greater mRNA levels, wherein the mRNA encodes a transcription factor, that is, a DNA-binding protein that is capable of binding to a DNA regulatory sequence and inducing transcription, and preferably, expression of a plant trait gene. Preferably, the mRNA expression level will be at least three-fold greater than that of the parental plant, or more preferably at least ten-fold greater mRNA levels compared to said parental plant, and most preferably at least fifty-fold greater compared to said parental plant.

Vectors, Promoters, and Expression Systems. The present invention includes recombinant constructs comprising one or more of the nucleic acid sequences herein. The constructs typically comprise a vector, such as a plasmid, a cosmid, a phage, a virus (e.g., a plant virus), a bacterial artificial chromosome (BAC), a yeast artificial chromosome (YAC), or the like, into which a nucleic acid sequence of the invention has been inserted, in a forward or reverse orientation. In a preferred aspect of this embodiment, the construct further comprises regulatory sequences, including, for example, a promoter, operably linked to the sequence. Large numbers of suitable vectors and promoters are known to those of skill in the art, and are commercially available.

General texts that describe molecular biological techniques useful herein, including the use and production of vectors, promoters and many other relevant topics, include Berger and Kimmel (1987) supra, Sambrook (1989) supra, and Ausubel (1997, 2000) supra. Any of the identified sequences can be incorporated into a cassette or vector, e.g., for expression in plants. A number of expression vectors suitable for stable transformation of plant cells or for the establishment of transgenic plants have been described including those described in Weissbach and Weissbach (1989) Methods for Plant Molecular Biology, Academic Press, and Gelvin et al. (1990) Plant Molecular Biology Manual, Kluwer Academic Publishers. Specific examples include those derived from a Ti plasmid of Agrobacterium tumefaciens, as well as those disclosed by Herrera-Estrella et al. (1983) Nature 303: 209, Bevan (1984) Nucleic Acids Res. 12: 8711-8721, Klee (1985) Bio/Technology 3: 637-642, for dicotyledonous plants.

Alternatively, non-Ti vectors can be used to transfer the DNA into monocotyledonous plants and cells by using free DNA delivery techniques. Such methods can involve, for example, the use of liposomes, electroporation, microprojectile bombardment, silicon carbide whiskers, and viruses. By using these methods transgenic plants such as wheat, rice (Christou (1991) Bio/Technology 9: 957-962) and corn (Gordon-Kamm (1990) Plant Cell 2: 603-618) can be produced. An immature embryo can also be a good target tissue for monocots for direct DNA delivery techniques by using the particle gun (Weeks et al. (1993) Plant Physiol. 102: 1077-1084; Vasil (1993) Bio/Technology 10: 667-674; Wan and Lemeaux (1994) Plant Physiol. 104: 37-48, and for Agrobacterium-mediated DNA transfer (Ishida et al. (1996) Nature Biotechnol. 14: 745-750).

Typically, plant transformation vectors include one or more cloned plant coding sequence (genomic or cDNA) under the transcriptional control of 5′ and 3′ regulatory sequences and a dominant selectable marker. Such plant transformation vectors typically also contain a promoter (e.g., a regulatory region controlling inducible or constitutive, environmentally- or developmentally-regulated, or cell- or tissue-specific expression), a transcription initiation start site, an RNA processing signal (such as intron splice sites), a transcription termination site, and/or a polyadenylation signal.

A potential utility for the transcription factor polynucleotides disclosed herein is the isolation of promoter elements from these genes that can be used to program expression in plants of any genes. Each transcription factor gene disclosed herein is expressed in a unique fashion, as determined by promoter elements located upstream of the start of translation, and additionally within an intron of the transcription factor gene or downstream of the termination codon of the gene. As is well known in the art, for a significant portion of genes, the promoter sequences are located entirely in the region directly upstream of the start of translation. In such cases, typically the promoter sequences are located within 2.0 kb of the start of translation, or within 1.5 kb of the start of translation, frequently within 1.0 kb of the start of translation, and sometimes within 0.5 kb of the start of translation.

The promoter sequences can be isolated according to methods known to one skilled in the art.

Examples of constitutive plant promoters which can be useful for expressing the TF sequence include: the cauliflower mosaic virus (CaMV) 35S promoter, which confers constitutive, high-level expression in most plant tissues (for example, Odell et al. (1985) Nature 313: 810-812); the nopaline synthase promoter (An et al. (1988) Plant Physiol. 88: 547-552); and the octopine synthase promoter (Fromm et al. (1989) Plant Cell 1: 977-984).

The transcription factors of the invention may be operably linked with a specific promoter that causes the transcription factor to be expressed in response to environmental, tissue-specific or temporal signals. A variety of plant gene promoters are known to regulate gene expression in response to environmental, hormonal, chemical, developmental signals, and in a tissue-active manner; many of these may be used for expression of a TF sequence in plants. Choice of a promoter is based largely on the phenotype of interest and is determined by such factors as tissue (e.g., seed, fruit, root, pollen, vascular tissue, flower, carpel, etc.), inducibility (e.g., in response to wounding, heat, cold, drought, light, pathogens, etc.), timing, developmental stage, and the like. Numerous known promoters have been characterized and can favorably be employed to promote expression of a polynucleotide of the invention in a transgenic plant or cell of interest. For example, tissue specific promoters include: seed-specific promoters (such as the napin, phaseolin or DC3 promoter described in U.S. Pat. No. 5,773,697), fruit-specific promoters that are active during fruit ripening, such as the dru 1 promoter (U.S. Pat. No. 5,783,393), or the 2A11 promoter (U.S. Pat. No. 4,943,674) and the tomato polygalacturonase promoter (Bird et al. (1988) Plant Mol. Biol. 11: 651-662), root-specific promoters, such as ARSK1, and those disclosed in U.S. Pat. Nos. 5,618,988, 5,837,848 and 5,905,186, epidermis-specific promoters, including CUT1 (Kunst et al. (1999) Biochem. Soc. Trans. 28: 651-654), pollen-active promoters such as PTA29, PTA26 and PTA 13 (U.S. Pat. No. 5,792,929), promoters active in vascular tissue (Ringli and Keller (1998) Plant Mol. Biol. 37: 977-988), flower-specific (Kaiser et al. (1995) Plant Mol. Biol. 28: 231-243), pollen (Baerson et al. (1994) Plant Mol. Biol. 26: 1947-1959), carpels (Ohl et al. (1990) Plant Cell 2: 837-848), pollen and ovules (Baerson et al. (1993) Plant Mol. Biol. 22: 255-267), auxin-inducible promoters (such as that described in van der Kop et al. (1999) Plant Mol. Biol. 39: 979-990 or Baumann et al. (1999) Plant Cell 11: 323-334), cytokinin-inducible promoter (Guevara-Garcia (1998) Plant Mol. Biol. 38: 743-753), promoters responsive to gibberellin (Shi et al. (1998) Plant Mol. Biol. 38: 1053-1060, Willmott et al. (1998) Plant Mol. Biol. 38: 817-825) and the like. Additional promoters are those that elicit expression in response to heat (Ainley et al. (1993) Plant Mol. Biol. 22: 13-23), light (e.g., the pea rbcS-3A promoter, described in Kuhlemeier et al. (1989) Plant Cell 1: 471-478, and the maize rbcS promoter, described in Schaffner and Sheen (1991) Plant Cell 3: 997-1012); wounding (e.g., wunI, described in Siebertz et al. (1989) Plant Cell 1: 961-968), pathogens (such as the PR-1 promoter described in Buchel et al. (1999) Plant Mol. Biol. 40: 387-396, and the PDF1.2 promoter described in Manners et al. (1998) Plant Mol. Biol. 38: 1071-1080), and chemicals such as methyl jasmonate or salicylic acid (Gatz (1997) Annu. Rev. Plant Physiol. Plant Mol. Biol. 48: 89-108). In addition, the timing of the expression can be controlled by using promoters such as those acting at senescence (Gan and Amasino (1995) Science 270: 1986-1988); or late seed development (Odell et al. (1994) Plant Physiol. 106: 447-458).

Plant expression vectors can also include RNA processing signals that can be positioned within, upstream or downstream of the coding sequence. In addition, the expression vectors can include additional regulatory sequences from the 3′-untranslated region of plant genes, e.g., a 3′ terminator region to increase mRNA stability of the mRNA, such as the PI-II terminator region of potato or the octopine or nopaline synthase 3′ terminator regions.

Additional Expression Elements. Specific initiation signals can aid in efficient translation of coding sequences. These signals can include, e.g., the ATG initiation codon and adjacent sequences. When a coding sequence, its initiation codon and upstream sequences are inserted into the appropriate expression vector, no additional translational control signals may be needed. However, in cases where only coding sequence (e.g., a mature protein coding sequence) or a portion thereof is inserted, exogenous transcriptional control signals including the ATG initiation codon can be separately provided. The initiation codon is provided in the correct reading frame to facilitate transcription. Exogenous transcriptional elements and initiation codons can be of various origins, both natural and synthetic. The efficiency of expression can be enhanced by the inclusion of enhancers appropriate to the cell system in use.

Expression Hosts. The present invention also relates to host cells which are transduced with vectors of the invention, and the production of polypeptides of the invention (including fragments thereof) by recombinant techniques. Host cells are genetically engineered (i.e., nucleic acids are introduced, e.g., transduced, transformed or transfected) with the vectors of this invention, which may be, for example, a cloning vector or an expression vector comprising the relevant nucleic acids herein. The vector is optionally a plasmid, a viral particle, a phage, a naked nucleic acid, etc. The engineered host cells can be cultured in conventional nutrient media modified as appropriate for activating promoters, selecting transformants, or amplifying the relevant gene. The culture conditions, such as temperature, pH and the like, are those previously used with the host cell selected for expression, and will be apparent to those skilled in the art and in the references cited herein, including, Sambrook (1989) supra and Ausubel (1997, 2000) supra.

The host cell can be a eukaryotic cell, such as a yeast cell, or a plant cell, or the host cell can be a prokaryotic cell, such as a bacterial cell. Plant protoplasts are also suitable for some applications. For example, the DNA fragments are introduced into plant tissues, cultured plant cells or plant protoplasts by standard methods including electroporation (Fromm et al. (1985) Proc. Natl. Acad. Sci. USA 82: 5824-5828), infection by viral vectors such as cauliflower mosaic virus (CaMV) (Hohn et al. (1982) Molecular Biology of Plant Tumors, Academic Press, New York, N.Y., pp. 549-560; U.S. Pat. No. 4,407,956), high velocity ballistic penetration by small particles with the nucleic acid either within the matrix of small beads or particles, or on the surface (Klein et al. (1987) Nature 327: 70-73), use of pollen as vector (WO 85/01856), or use of Agrobacterium tumefaciens or A. rhizogenes carrying a T-DNA plasmid in which DNA fragments are cloned. The T-DNA plasmid is transmitted to plant cells upon infection by Agrobacterium tumefaciens, and a portion is stably integrated into the plant genome (Horsch et al. (1984) Science 233: 496-498; Fraley et al. (1983) Proc. Natl. Acad. Sci. USA 80: 4803-4807).

The cell can include a nucleic acid of the invention that encodes a polypeptide, wherein the cell expresses a polypeptide of the invention. The cell can also include vector sequences, or the like. Furthermore, cells and transgenic plants that include any polypeptide or nucleic acid above or throughout this specification, e.g., produced by transduction of a vector of the invention, are an additional feature of the invention.

For long-term, high-yield production of recombinant proteins, stable expression can be used. Host cells transformed with a nucleotide sequence encoding a polypeptide of the invention are optionally cultured under conditions suitable for the expression and recovery of the encoded protein from cell culture. The protein or fragment thereof produced by a recombinant cell may be secreted, membrane-bound, or contained intracellularly, depending on the sequence and/or the vector used. As will be understood by those of skill in the art, expression vectors containing polynucleotides encoding mature proteins of the invention can be designed with signal sequences which direct secretion of the mature polypeptides through a prokaryotic or eukaryotic cell membrane.

Production of Transgenic Plants

Modification of Traits. The polynucleotides of the invention are favorably employed to produce transgenic plants with various traits, or characteristics, that have been modified in a desirable manner, e.g., to improve the seed characteristics of a plant. For example, alteration of expression levels or patterns (e.g., spatial or temporal expression patterns) of one or more of the transcription factors (or transcription factor homologs) of the invention, as compared with the levels of the same protein found in a wild-type plant, can be used to modify a plant's traits. An illustrative example of trait modification, improved characteristics, by altering expression levels of a particular transcription factor is described further in the Examples and the Sequence Listing.

Arabidopsis as a model system. Arabidopsis thaliana is the object of rapidly growing attention as a model for genetics and metabolism in plants. Arabidopsis has a small genome, and well-documented studies are available. It is easy to grow in large numbers and mutants defining important genetically controlled mechanisms are either available, or can readily be obtained. Various methods to introduce and express isolated homologous genes are available (Koncz et al., editors, Methods in Arabidopsis Research (1992) World Scientific, New Jersey N.J., in “Preface”). Because of its small size, short life cycle, obligate autogamy and high fertility, Arabidopsis is also a choice organism for the isolation of mutants and studies in morphogenetic and development pathways, and control of these pathways by transcription factors (Koncz (1992) supra, p. 72). A number of studies introducing transcription factors into A. thaliana have demonstrated the utility of this plant for understanding the mechanisms of gene regulation and trait alteration in plants (for example, Koncz (1992) supra, and U.S. Pat. No. 6,417,428).

Arabidopsis genes in transgenic plants. Expression of genes encoding transcription factors that modify expression of endogenous genes, polynucleotides, and proteins are well known in the art. In addition, transgenic plants comprising isolated polynucleotides encoding transcription factors may also modify expression of endogenous genes, polynucleotides, and proteins. Examples include Peng et al. (1997) et al. Genes and Development 11: 3194-3205, and Peng et al. (1999) Nature 400: 256-261. In addition, many others have demonstrated that an Arabidopsis transcription factor expressed in an exogenous plant species elicits the same or very similar phenotypic response (for example, Fu et al. (2001) Plant Cell 13: 1791-1802; Nandi et al. (2000) Curr. Biol. 10: 215-218; Coupland (1995) Nature 377: 482-483; and Weigel and Nilsson (1995) Nature 377: 482-500).

Homologous genes introduced into transgenic plants. Homologous genes that may be derived from any plant, or from any source whether natural, synthetic, semi-synthetic or recombinant, and that share significant sequence identity or similarity to those provided by the present invention, may be introduced into plants, for example, crop plants, to confer desirable or improved traits. Consequently, transgenic plants may be produced that comprise a recombinant expression vector or cassette with a promoter operably linked to one or more sequences homologous to presently disclosed sequences. The promoter may be, for example, a plant or viral promoter.

The invention thus provides for methods for preparing transgenic plants, and for modifying plant traits. These methods include introducing into a plant a recombinant expression vector or cassette comprising a functional promoter operably linked to one or more sequences homologous to presently disclosed sequences. Plants and kits for producing these plants that result from the application of these methods are also encompassed by the present invention.

Transcription factors of interest for the modification of plant traits. Currently, the existence of a series of maturity groups for different latitudes represents a major barrier to the introduction of new valuable traits. Any trait (e.g. increased tolerance to an abiotic or biotic stress) has to be bred into each of the different maturity groups separately, a laborious and costly exercise. The availability of single strain, which could be grown at any latitude, would therefore greatly increase the potential for introducing new traits to crop species such as soybean and cotton.

For the specific effects, traits and utilities conferred to plants, one or more transcription factor genes of the present invention may be used to increase or decrease, or improve or prove deleterious to a given trait. For example, knocking out a transcription factor gene that naturally occurs in a plant, or suppressing the gene (with, for example, antisense suppression), may cause decreased tolerance to an osmotic stress relative to non-transformed or wild-type plants. By overexpressing this gene, the plant may experience increased tolerance to the same stress. More than one transcription factor gene may be introduced into a plant, either by transforming the plant with one or more vectors comprising two or more transcription factors, or by selective breeding of plants to yield hybrid crosses that comprise more than one introduced transcription factor.

Genes, traits and utilities that affect plant characteristics. Plant transcription factors can modulate gene expression, and, in turn, be modulated by the environmental experience of a plant. Significant alterations in a plant's environment invariably result in a change in the plant's transcription factor gene expression pattern. Altered transcription factor expression patterns generally result in phenotypic changes in the plant. Transcription factor gene product(s) in transgenic plants then differ(s) in amounts or proportions from that found in wild-type or non-transformed plants, and those transcription factors likely represent polypeptides that are used to alter the response to the environmental change. By way of example, it is well accepted in the art that analytical methods based on altered expression patterns may be used to screen for phenotypic changes in a plant far more effectively than can be achieved using traditional methods.

Plants overexpressing members of the G1792 clade of transcription factor polypeptides, including sequences from diverse species of monocots and dicots, such as Arabidopsis thaliana polypeptides G1792, G1791, G1795 and G30, Oryza sativa polypeptide G3381, and Glycine max polypeptide G3520, were shown to be more tolerant to low nitrogen conditions than control plants (Example VIII).

The invention also provides polynucleotides that encode G1792 clade polypeptides, fragments thereof, conserved domains thereof, paralogs, orthologs, equivalogs, and fragments thereof. Examples of these sequences are listed in the Sequence Listing, and due to the high degree of structural similarity to the sequences of the invention, it is expected that many of the sequences for which data have not been generated will also function to increase abiotic stress and/or low nitrogen tolerance. The invention also encompasses the complements of the polynucleotides. The polynucleotides are also useful for screening libraries of molecules or compounds for specific binding and for identifying other sequences of G1792 clade member by identifying orthologs having similar sequences, particularly in the conserved domains.

Antisense and Co-suppression. In addition to expression of the nucleic acids of the invention as gene replacement or plant phenotype modification nucleic acids, the nucleic acids are also useful for sense and anti-sense suppression of expression, e.g., to down-regulate expression of a nucleic acid of the invention, e.g., as a further mechanism for modulating plant phenotype. That is, the nucleic acids of the invention, or subsequences or anti-sense sequences thereof, can be used to block expression of naturally occurring homologous nucleic acids. A variety of sense and anti-sense technologies are known in the art, e.g., as set forth in Lichtenstein and Nellen (1997) Antisense Technology: A Practical Approach IRL Press at Oxford University Press, Oxford, U.K. Antisense regulation is also described in Crowley et al. (1985) Cell 43: 633-641; Rosenberg et al. (1985) Nature 313: 703-706; Preiss et al. (1985) Nature 313: 27-32; Melton (1985) Proc. Natl. Acad. Sci. USA 82: 144-148; Izant and Weintraub (1985) Science 229: 345-352; and Kim and Wold (1985) Cell 42: 129-138. Additional methods for antisense regulation are known in the art. Antisense regulation has been used to reduce or inhibit expression of plant genes in, for example in European Patent Publication No. 271988. Antisense RNA may be used to reduce gene expression to produce a visible or biochemical phenotypic change in a plant (Smith et al. (1988) Nature 334: 724-726; Smith et al. (1990) Plant Mol. Biol. 14: 369-379). In general, sense or anti-sense sequences are introduced into a cell, where they are optionally amplified, for example, by transcription. Such sequences include both simple oligonucleotide sequences and catalytic sequences such as ribozymes.

For example, a reduction or elimination of expression (i.e., a “knock-out”) of a transcription factor or transcription factor homolog polypeptide in a transgenic plant, e.g., to modify a plant trait, can be obtained by introducing an antisense construct corresponding to the polypeptide of interest as a cDNA. For antisense suppression, the transcription factor or homolog cDNA is arranged in reverse orientation (with respect to the coding sequence) relative to the promoter sequence in the expression vector. The introduced sequence need not be the full length cDNA or gene, and need not be identical to the cDNA or gene found in the plant type to be transformed. Typically, the antisense sequence need only be capable of hybridizing to the target gene or RNA of interest. Thus, where the introduced sequence is of shorter length, a higher degree of homology to the endogenous transcription factor sequence will be needed for effective antisense suppression. While antisense sequences of various lengths can be utilized, preferably, the introduced antisense sequence in the vector will be at least 30 nucleotides in length, and improved antisense suppression will typically be observed as the length of the antisense sequence increases. Preferably, the length of the antisense sequence in the vector will be greater than 100 nucleotides. Transcription of an antisense construct as described results in the production of RNA molecules that are the reverse complement of mRNA molecules transcribed from the endogenous transcription factor gene in the plant cell.

Suppression of endogenous transcription factor gene expression can also be achieved using a ribozyme. Ribozymes are RNA molecules that possess highly specific endoribonuclease activity. The production and use of ribozymes are disclosed in U.S. Pat. No. 4,987,071 and U.S. Pat. No. 5,543,508. Synthetic ribozyme sequences including antisense RNAs can be used to confer RNA cleaving activity on the antisense RNA, such that endogenous mRNA molecules that hybridize to the antisense RNA are cleaved, which in turn leads to an enhanced antisense inhibition of endogenous gene expression.

Vectors in which RNA encoded by a transcription factor or transcription factor homolog cDNA is over-expressed can also be used to obtain co-suppression of a corresponding endogenous gene, for example, in the manner disclosed in U.S. Pat. No. 5,231,020. Such co-suppression (also termed sense suppression) does not require that the entire transcription factor cDNA be introduced into the plant cells, nor does it require that the introduced sequence be exactly identical to the endogenous transcription factor gene of interest. However, as with antisense suppression, the suppressive efficiency will be enhanced as specificity of hybridization is increased, e.g., as the introduced sequence is lengthened, and/or as the sequence similarity between the introduced sequence and the endogenous transcription factor gene is increased.

Vectors expressing an untranslatable form of the transcription factor mRNA (e.g., sequences comprising one or more stop codon, or nonsense mutation) can also be used to suppress expression of an endogenous transcription factor, thereby reducing or eliminating its activity and modifying one or more traits. Methods for producing such constructs are described in U.S. Pat. No. 5,583,021. Preferably, such constructs are made by introducing a premature stop codon into the transcription factor gene. Alternatively, a plant trait can be modified by gene silencing using double-stranded RNA (Sharp (1999) Genes and Development 13: 139-141). Another method for abolishing the expression of a gene is by insertion mutagenesis using the T-DNA of Agrobacterium tumefaciens. After generating the insertion mutants, the mutants can be screened to identify those containing the insertion in a transcription factor or transcription factor homolog gene. Plants containing a single transgene insertion event at the desired gene can be crossed to generate homozygous plants for the mutation. Such methods are well known to those of skill in the art (for example, in Koncz et al. (1992) supra).

Suppression of endogenous transcription factor gene expression can also be achieved using RNA interference, or RNAi. RNAi is a post-transcriptional, targeted gene-silencing technique that uses double-stranded RNA (dsRNA) to incite degradation of messenger RNA (mRNA) containing the same sequence as the dsRNA (Constans (2002) The Scientist 16:36). Small interfering RNAs, or siRNAs are produced in at least two steps: an endogenous ribonuclease cleaves longer dsRNA into shorter, 21-23 nucleotide-long RNAs. The siRNA segments then mediate the degradation of the target mRNA (Zamore (2001) Nature Struct. Biol 8: 746-50). RNAi has been used for gene function determination in a manner similar to antisense oligonucleotides (Constans (2002) supra). Expression vectors that continually express siRNAs in transiently and stably-transfected cells have been engineered to express small hairpin RNAs (shRNAs), which get processed in vivo into siRNAs-like molecules capable of carrying out gene-specific silencing (Brummelkamp et al. (2002) Science 296:550-553, and Paddison et al. (2002) Genes & Dev. 16:948-958). Post-transcriptional gene silencing by double-stranded RNA is discussed in further detail by Hammond et al. (2001) Nature Rev Gen 2: 110-119, Fire et al. (1998) Nature 391: 806-811 and Timmons and Fire (1998) Nature 395: 854.

Alternatively, a plant phenotype can be altered by eliminating an endogenous gene, such as a transcription factor or transcription factor homolog, e.g., by homologous recombination (Kempin et al. (1997) Nature 389: 802-803).

A plant trait can also be modified by using the Cre-lox system (for example, as described in U.S. Pat. No. 5,658,772). A plant genome can be modified to include first and second lox sites that are then contacted with a Cre recombinase. If the lox sites are in the same orientation, the intervening DNA sequence between the two sites is excised. If the lox sites are in the opposite orientation, the intervening sequence is inverted.

The polynucleotides and polypeptides of this invention can also be expressed in a plant in the absence of an expression cassette by manipulating the activity or expression level of the endogenous gene by other means, such as, for example, by ectopically expressing a gene by T-DNA activation tagging (Ichikawa et al. (1997) Nature 390 698-701; Kakimoto et al. (1996) Science 274: 982-985). This method entails transforming a plant with a gene tag containing multiple transcriptional enhancers and once the tag has inserted into the genome, expression of a flanking gene coding sequence becomes deregulated. In another example, the transcriptional machinery in a plant can be modified so as to increase transcription levels of a polynucleotide of the invention (for example, in PCT Publications WO 96/06166 and WO 98/53057 which describe the modification of the DNA-binding specificity of zinc finger proteins by changing particular amino acids in the DNA-binding motif).

The transgenic plant can also include the machinery necessary for expressing or altering the activity of a polypeptide encoded by an endogenous gene, for example, by altering the phosphorylation state of the polypeptide to maintain it in an activated state.

Transgenic plants (or plant cells, or plant explants, or plant tissues) incorporating the polynucleotides of the invention and/or expressing the polypeptides of the invention can be produced by a variety of well established techniques as described above. Following construction of a vector, most typically an expression cassette, including a polynucleotide, e.g., encoding a transcription factor or transcription factor homolog, of the invention, standard techniques can be used to introduce the polynucleotide into a plant, a plant cell, a plant explant or a plant tissue of interest. Optionally, the plant cell, explant or tissue can be regenerated to produce a transgenic plant.

The plant can be any higher plant, including gymnosperms, monocotyledonous and dicotyledonous plants. Suitable protocols are available for Leguminosae (alfalfa, soybean, clover, etc.), Umbelliferae (carrot, celery, parsnip), Cruciferae (cabbage, radish, rapeseed, broccoli, etc.), Curcurbitaceae (melons and cucumber), Gramineae (wheat, corn, rice, barley, millet, etc.), Solanaceae (potato, tomato, tobacco, peppers, etc.), and various other crops. Examples of these protocols are described in Ammirato et al. eds., (1984) Handbook of Plant Cell Culture—Crop Species, Macmillan Publ. Co., New York N.Y.; Shimamoto et al. (1989) Nature 338: 274-276; Fromm et al. (1990) Bio/Technol. 8: 833-839; and Vasil et al. (1990) Bio/Technol. 8: 429-434.

Transformation and regeneration of both monocotyledonous and dicotyledonous plant cells are now routine, and the selection of the most appropriate transformation technique will be determined by the practitioner. The choice of method will vary with the type of plant to be transformed; those skilled in the art will recognize the suitability of particular methods for given plant types. Suitable methods can include, but are not limited to: electroporation of plant protoplasts; liposome-mediated transformation; polyethylene glycol (PEG) mediated transformation; transformation using viruses; micro-injection of plant cells; micro-projectile bombardment of plant cells; vacuum infiltration; and Agrobacterium tumefaciens-mediated transformation. Transformation means introducing a nucleotide sequence into a plant in a manner to cause stable or transient expression of the sequence.

Successful examples of the modification of plant characteristics by transformation with cloned sequences which serve to illustrate the current knowledge in this field of technology, and which are herein incorporated by reference, include: U.S. Pat. Nos. 5,571,706; 5,677,175; 5,510,471; 5,750,386; 5,597,945; 5,589,615; 5,750,871; 5,268,526; 5,780,708; 5,538,880; 5,773,269; 5,736,369 and 5,610,042.

Following transformation, plants are preferably selected using a dominant selectable marker incorporated into the transformation vector. Typically, such a marker will confer antibiotic or herbicide resistance on the transformed plants, and selection of transformants can be accomplished by exposing the plants to appropriate concentrations of the antibiotic or herbicide.

After transformed plants are selected and grown to maturity, those plants—showing a modified trait are identified. The modified trait can be any of those traits described above. Additionally, to confirm that the modified trait is due to changes in expression levels or activity of the polypeptide or polynucleotide of the invention can be determined by analyzing mRNA expression using Northern blots, RT-PCR or microarrays, or protein expression using immunoblots or Western blots or gel shift assays.

Integrated Systems—Sequence Identity. In addition to providing compositions and methods to improve plant traits, the present invention may be an integrated system, computer or computer readable medium that comprises an instruction set for determining the identity of one or more sequences in a database. In addition, the instruction set can be used to generate or identify sequences that meet any specified criteria. Furthermore, the instruction set may be used to associate or link certain functional benefits, such improved characteristics, with one or more identified sequence.

For example, the instruction set can include, e.g., a sequence comparison or other alignment program, e.g., an available program such as, for example, the Wisconsin Package Version 10.0, such as BLAST, FASTA, PILEUP, FINDPATTERNS or the like (GCG, Madison, Wis.). Public sequence databases such as GenBank, EMBL, Swiss-Prot and PIR or private sequence databases such as PHYTOSEQ sequence database (Incyte Genomics, Wilmington, Del.) can be searched.

Alignment of sequences for comparison can be conducted by the local homology algorithm of Smith and Waterman (1981) Adv. Appl. Mach. 2: 482-489, by the homology alignment algorithm of Needleman and Wunsch (1970) J. Mol. Biol. 48: 443-453, by the search for similarity method of Pearson and Lipman (1988) Proc. Natl. Acad. Sci. USA 85: 2444-2448, by computerized implementations of these algorithms. After alignment, sequence comparisons between two (or more) polynucleotides or polypeptides are typically performed by comparing sequences of the two sequences over a comparison window to identify and compare local regions of sequence similarity. The comparison window can be a segment of at least about 20 contiguous positions, usually about 50 to about 200, more usually about 100 to about 150 contiguous positions. A description of the method is provided in Ausubel et al. (1997, 2000) supra.

A variety of methods for determining sequence relationships can be used, including manual alignment and computer assisted sequence alignment and analysis. This later approach is a preferred approach in the present invention, due to the increased throughput afforded by computer assisted methods. As noted above, a variety of computer programs for performing sequence alignment are available, or can be produced by one of skill.

One example algorithm that is suitable for determining percent sequence identity and sequence similarity is the BLAST algorithm, which is described in Altschul et al. (1990) supra. Software for performing BLAST analyses is publicly available, e.g., through the National Library of Medicine's National Center for Biotechnology Information (National Institutes of Health US government website at www.ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990, 1993) supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100 μM=5, N=−4, and a comparison of both strands. For amino acid sequences, the BLASTP program uses as defaults a wordlength (W) of 3, an expectation (E) of 10, and the BLOSUM62 scoring matrix (Henikoff and Henikoff (1992) Proc. Natl. Acad. Sci. USA 89: 10915-10919). Unless otherwise indicated, “sequence identity” here refers to the % sequence identity generated from a tblastx using the NCBI version of the algorithm at the default settings using gapped alignments with the filter “off” (for example, at the NIH website at www.ncbi.nlm.nih.gov, supra).

In addition to calculating percent sequence identity, the BLAST algorithm also performs a statistical analysis of the similarity between two sequences (for example, Karlin and Altschul (1993) Proc. Natl. Acad. Sci. USA 90: 5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence (and, therefore, in this context, homologous) if the smallest sum probability in a comparison of the test nucleic acid to the reference nucleic acid is less than about 0.1, or less than about 0.01, and or even less than about 0.001. An additional example of a useful sequence alignment algorithm is PILEUP. PILEUP creates a multiple sequence alignment from a group of related sequences using progressive, pairwise alignments. The program can align, for example, up to 300 sequences of a maximum length of 5,000 letters.

The integrated system, or computer typically includes a user input interface allowing a user to selectively view one or more sequence records corresponding to the one or more character strings, as well as an instruction set which aligns the one or more character strings with each other or with an additional character string to identify one or more region of sequence similarity. The system may include a link of one or more character strings with a particular phenotype or gene function. Typically, the system includes a user readable output element that displays an alignment produced by the alignment instruction set.

The methods of this invention can be implemented in a localized or distributed computing environment. In a distributed environment, the methods may be implemented on a single computer comprising multiple processors or on a multiplicity of computers. The computers can be linked, e.g. through a common bus, but more preferably the computer(s) are nodes on a network. The network can be a generalized or a dedicated local or wide-area network and, in certain preferred embodiments, the computers may be components of an intra-net or an internet.

Thus, the invention provides methods for identifying a sequence similar or homologous to one or more polynucleotides as noted herein, or one or more target polypeptides encoded by the polynucleotides, or otherwise noted herein and may include linking or associating a given plant phenotype or gene function with a sequence. In the methods, a sequence database is provided (locally or across an inter or intra net) and a query is made against the sequence database using the relevant sequences herein and associated plant phenotypes or gene functions.

Any sequence herein can be entered into the database, before or after querying the database. This provides for both expansion of the database and, if done before the querying step, for insertion of control sequences into the database. The control sequences can be detected by the query to ensure the general integrity of both the database and the query. As noted, the query can be performed using a web browser based interface. For example, the database can be a centralized public database such as those noted herein, and the querying can be done from a remote terminal or computer across an internet or intranet.

Any sequence herein can be used to identify a similar, homologous, paralogous, or orthologous sequence in another plant. This provides means for identifying endogenous sequences in other plants that may be useful to alter a trait of progeny plants, which results from crossing two plants of different strain. For example, sequences that encode an ortholog of any of the sequences herein that naturally occur in a plant with a desired trait can be identified using the sequences disclosed herein. The plant is then crossed with a second plant of the same species but which does not have the desired trait to produce progeny which can then be used in further crossing experiments to produce the desired trait in the second plant. Therefore the resulting progeny plant contains no transgenes; expression of the endogenous sequence may also be regulated by treatment with a particular chemical or other means, such as EMR. Some examples of such compounds well known in the art include: ethylene; cytokinins; phenolic compounds, which stimulate the transcription of the genes needed for infection; specific monosaccharides and acidic environments that potentiate vir gene induction; acidic polysaccharides which induce one or more chromosomal genes; and opines; other mechanisms include light or dark treatment (reviews of such treatments appears in Winans (1992) Microbiol. Rev. 56: 12-31; Eyal et al. (1992) Plant Mol. Biol. 19: 589-599; Chrispeels et al. (2000) Plant Mol. Biol. 42: 279-290; and Piazza et al. (2002) Plant Physiol. 128: 1077-1086).

### EXAMPLES

This invention is not limited to the particular devices, machines, materials and methods described. Although particular embodiments are described, equivalent embodiments may be used to practice the invention. The examples below are provided to enable the subject invention and are not included for the purpose of limiting the invention.

The invention being generally described will be more readily understood by reference to the following examples, which are included merely for purposes of illustration of certain aspects and embodiments of the present invention and are not intended to limit the invention. It will be recognized by one of skill in the art that a transcription factor associated with a particular first trait may also be associated with at least one other, unrelated and inherent second trait which was not predicted by the first trait.

### Example I

### Full Length Gene Identification and Cloning

Arabidopsis transcription factor clones used in these studies were made in one of three ways: isolation from a library, amplification from cDNA, or amplification from genomic DNA. The ends of the Arabidopsis transcription factor coding sequences were generally confirmed by RACE PCR or by comparison with public cDNA sequences before cloning.

Putative transcription factor sequences (genomic or ESTs) related to known transcription factors were identified in the Arabidopsis thaliana GenBank database using the tblastn sequence analysis program using default parameters and a P-value cutoff threshold of −4 or −5 or lower, depending on the length of the query sequence. Putative transcription factor sequence hits were then screened to identify those containing particular sequence strings. If the sequence hits contained such sequence strings, the sequences were confirmed as transcription factors.

Alternatively, Arabidopsis thaliana cDNA libraries derived from different tissues or treatments, or genomic libraries were screened to identify novel members of a transcription family using a low stringency hybridization approach. Probes were synthesized using gene specific primers in a standard PCR reaction (annealing temperature 60° C.) and labeled with ³²P dCTP using the High Prime DNA Labeling Kit (Roche Diagnostics Corp., Indianapolis, Ind.). Purified radiolabelled probes were added to filters immersed in Church hybridization medium (0.5 M NaPO₄ pH 7.0, 7% SDS, 1% w/v bovine serum albumin) and hybridized overnight at 60° C. with shaking. Filters were washed two times for 45 to 60 minutes with 1×SSC, 1% SDS at 60° C.

To identify additional sequence 5′ or 3′ of a partial cDNA sequence in a cDNA library, 5′ and 3′ rapid amplification of cDNA ends (RACE) was performed using the MARATHON cDNA amplification kit (Clontech, Palo Alto, Calif.). Generally, the method entailed first isolating poly(A) mRNA, performing first and second strand cDNA synthesis to generate double stranded cDNA, blunting cDNA ends, followed by ligation of the MARATHON Adaptor to the cDNA to form a library of adaptor-ligated ds cDNA.

Gene-specific primers were designed to be used along with adaptor specific primers for both 5′ and 3′ RACE reactions. Nested primers, rather than single primers, were used to increase PCR specificity. Using 5′ and 3′ RACE reactions, 5′ and 3′ RACE fragments were obtained, sequenced and cloned. The process can be repeated until 5′ and 3′ ends of the full-length gene were identified. Then the full-length cDNA was generated by PCR using primers specific to 5′ and 3′ ends of the gene by end-to-end PCR.

Clones of transcription factor orthologs from rice, maize, and soybean presented in this report were all made by amplification from cDNA. The ends of the coding sequences were predicted based on homology to Arabidopsis or by comparison to public and proprietary cDNA sequences; RACE PCR was not done to confirm the ends of the coding sequences. For cDNA amplification, we used KOD Hot Start DNA Polymerase (Novagen), in combination with 1M betaine and 3% DMSO. This protocol was found to be successful in amplifying cDNA from GC-rich species such as rice and corn, along with some non-GC-rich species such as soybean and tomato, where traditional PCR protocols failed. Primers were designed using at least 30 bases specific to the target sequence, and were designed close to, or overlapping, the start and stop codons of the predicted coding sequence.

Clones were fully sequenced. In the case of rice, high-quality public genomic sequence is available for comparison, and clones with sequence changes that result in changes in amino acid sequence of the encoded protein were rejected. For corn and soy, however, it was often unclear whether sequence differences represented an error or polymorphism in the source sequence or a PCR error in the clone. Therefore, in the cases where the sequence of the clone we obtained differed from the source sequence, a second clone was created from an independent PCR reaction. If the sequences of the two clones agreed, then the clone was accepted as a legitimate sequence variant.

### Example II

### Construction of Expression Vectors

The sequence was amplified from a genomic or cDNA library using primers specific to sequences upstream and downstream of the coding region. The expression vector was pMEN20 or pMEN65 (SEQ ID NO: 68), which are both derived from pMON316 (Sanders et al. (1987) Nucleic Acids Res. 15:1543-1558) and contain the CaMV 35S promoter to express transgenes (pMEN20 is an earlier version of pMEN65 in which the kanamycin resistance gene is driven by the 35S promoter rather than the nos promoter. It is the base vector for P5381 and P5375). To clone the sequence into the vector, both pMEN20 and the amplified DNA fragment were digested separately with SalI and NotI restriction enzymes at 37° C. for 2 hours. The digestion products were subject to electrophoresis in a 0.8% agarose gel and visualized by ethidium bromide staining. The DNA fragments containing the sequence and the linearized plasmid were excised and purified by using a QIAQUICK gel extraction kit (Qiagen, Valencia, Calif.). The fragments of interest were ligated at a ratio of 3:1 (vector to insert). Ligation reactions using T4 DNA ligase (New England Biolabs, Beverly Mass.) were carried out at 16° C. for 16 hours. The ligated DNAs were transformed into competent cells of the E. coli strain DH5alpha by using the heat shock method. The transformations were plated on LB plates containing 50 mg/l kanamycin (Sigma Chemical Co. St. Louis Mo.). Individual colonies were grown overnight in five milliliters of LB broth containing 50 mg/l kanamycin at 37° C. Plasmid DNA was purified by using Qiaquick Mini Prep kits (Qiagen, Valencia, Calif.).

Two-component vectors. P5381 (pMEN53; SEQ ID NO: 64) is the 2-component base vector that is used to express genes under the control of the LexA operator. It contains eight tandem LexA operators from plasmid p8op-lacZ (Clontech) followed by a polylinker. The plasmid carries a sulfonamide resistance gene driven by the 35S promoter.

GAL4 fusion vectors. P21195 (SEQ ID NO: 65) is the backbone vector for creation of N-terminal GAL4 activation domain protein fusions. It was created by inserting the GAL4 activation domain into the BglII and KpnI sites of pMEN65. To create gene fusions, the transcription factor gene of interest is amplified using a primer that starts at the second amino acid and has added the KpnI or SalI and NotI sites. The PCR product is then cloned into the KpnI or SalI and NotI sites of P21195, taking care to maintain the reading frame.

P21378 (SEQ ID NO: 66) was constructed to serve as a backbone vector for creation of C-terminal GAL4 activation domain fusions. However, P5425 was also used as a backbone construct. P21378 was constructed by amplification of the GAL4 activation domain and insertion of this domain into the NotI and XbaI sites of pMEN65. To create gene fusions, the transcription factor gene of interest is amplified using a 3′ primer that ends at the last amino acid codon before the stop codon. The PCR product can then be cloned into the SalI and NotI sites.

P5425 (also called pMEN201) is a derivative of pMEN20 that carries a CBF1:GAL4 fusion. To construct other GAL4 fusions, the CBF1 gene was removed with SalI or KpnI and EcoRI. The gene of interest was amplified using a 3′ primer that ended at the last amino acid codon before the stop codon and contained an EcoRI or MfeI site. The product was inserted into these SalI or KpnI and EcoRI sites, taking care to maintain the reading frame.

### Example III

### Transformation of Agrobacterium with the Expression Vector

Direct promoter fusion. After the plasmid vector containing the gene was constructed, the vector was used to transform Agrobacterium tumefaciens cells expressing the gene products. The stock of Agrobacterium tumefaciens cells for transformation was made as described by Nagel et al. (1990) FEMS Microbiol Letts. 67: 325-328. Agrobacterium strain ABI was grown in 250 ml LB medium (Sigma Chemical Co., St. Louis, Mo.) overnight at 28° C. with shaking until an absorbance over 1 cm at 600 nm (A₆₀₀) of 0.5-1.0 was reached. Cells were harvested by centrifugation at 4,000×g for 15 minutes at 4° C. Cells were then resuspended in 250 μl chilled buffer (1 mM HEPES, pH adjusted to 7.0 with KOH). Cells were centrifuged again as described above and resuspended in 125 μl chilled buffer. Cells were then centrifuged and resuspended two more times in the same HEPES buffer as described above at a volume of 100 μl and 750 μl, respectively. Resuspended cells were then distributed into 40 μl aliquots, quickly frozen in liquid nitrogen, and stored at −80° C.

Agrobacterium cells were transformed with plasmids prepared as described above following the protocol described by Nagel et al. (supra). For each DNA construct to be transformed, 50-100 ng DNA (generally resuspended in 10 mM Tris-HCl, 1 mM EDTA, pH 8.0) was mixed with 40 μl of Agrobacterium cells. The DNA/cell mixture was then transferred to a chilled cuvette with a 2 mm electrode gap and subject to a 2.5 kV charge dissipated at 25 μF and 200 μF using a Gene Pulser II apparatus (Bio-Rad, Hercules, Calif.). After electroporation, cells were immediately resuspended in 1.0 ml LB and allowed to recover without antibiotic selection for 2-4 hours at 28° C. in a shaking incubator. After recovery, cells were plated onto selective medium of LB broth containing 100 μg/ml spectinomycin (Sigma Chemical Co., St. Louis, Mo.) and incubated for 24-48 hours at 28° C. Single colonies were then picked and inoculated in fresh medium. The presence of the plasmid construct was verified by PCR amplification and sequence analysis.

The two-component expression system. For the two-component system, two separate constructs were used: Promoter::LexA-GAL4TA and opLexA::TF. The first of these (Promoter::LexA-GAL4TA) comprised a desired promoter cloned in front of a LexA DNA binding domain fused to a GAL4 activation domain. The construct vector backbone (pMEN48, also known as P5375, SEQ ID NO: 67) also carried a kanamycin resistance marker, along with an opLexA::GFP reporter. Transgenic lines were obtained containing this first component, and a line was selected that showed reproducible expression of the reporter gene in the desired pattern through a number of generations. A homozygous population was established for that line, and the population was supertransformed with the second construct (opLexA::TF) carrying the TF of interest cloned behind a LexA operator site. This second construct vector backbone is pMEN53 (P5381, SEQ ID NO: 64), noted above.

### Example IV

### Transformation of Arabidopsis Plants with Agrobacterium tumefaciens

Agrobacterium strain ABI was used for all plant transformations. This strain is chloramphenicol, kanamycin and gentamicin resistant. After transformation of Agrobacterium tumefaciens with plasmid vectors containing the gene, single Agrobacterium colonies were identified, propagated, and used to transform Arabidopsis plants. Briefly, 500 ml cultures of LB medium containing 50 mg/l kanamycin were inoculated with the colonies and grown at 28° C. with shaking for 2 days until an optical absorbance at 600 nm wavelength over 1 cm (A₆₀₀) of >2.0 is reached. Cells were then harvested by centrifugation at 4,000×g for 10 minutes, and resuspended in infiltration medium (½× Murashige and Skoog salts (Sigma Chemical Co., St. Louis, Mo.), 1× Gamborg's B-5 vitamins (Sigma Chemical Co., St. Louis, Mo.), 5.0% (w/v) sucrose, 0.044 μM benzylamino purine (Sigma Chemical Co., St. Louis, Mo.), 200 μl/l Silwet L-77 (Lehle Seeds, Round Rock, Tex.) until an A₆₀₀ of 0.8 was reached).

Prior to transformation, Arabidopsis thaliana seeds (ecotype Columbia) were sown at a density of ˜10 plants per 4″ pot onto Pro-Mix BX potting medium (Hummert International) covered with fiberglass mesh (18 mm×16 mm). Plants were grown under continuous illumination (50-75 μE/m²/second) at 22-23° C. with 65-70% relative humidity. After about 4 weeks, primary inflorescence stems (bolts) are cut off to encourage growth of multiple secondary bolts. After flowering of the mature secondary bolts, plants were prepared for transformation by removal of all siliques and opened flowers.

The pots were then immersed upside down in the mixture of Agrobacterium infiltration medium as described above for 30 seconds, and placed on their sides to allow draining into a 1′×2′ flat surface covered with plastic wrap. After 24 h, the plastic wrap was removed and pots are turned upright. The immersion procedure was repeated one week later, for a total of two immersions per pot. Seeds were then collected from each transformation pot and analyzed following the protocol described below.

### Example V

### Identification of Arabidopsis Primary Transformants

Seeds collected from the transformation pots were sterilized essentially as follows. Seeds were dispersed into in a solution containing 0.1% (v/v) Triton X-100 (Sigma Chemical Co., St. Louis, Mo.) and sterile water and washed by shaking the suspension for 20 minutes. The wash solution was then drained and replaced with fresh wash solution to wash the seeds for 20 minutes with shaking. After removal of the ethanol/detergent solution, a solution containing 0.1% (v/v) Triton X-100 and 30% (v/v) bleach (CLOROX; Clorox Corp. Oakland, Calif.) was added to the seeds, and the suspension was shaken for 10 minutes. After removal of the bleach/detergent solution, seeds were then washed five times in sterile distilled water. The seeds were stored in the last wash water at 4° C. for 2 days in the dark before being plated onto antibiotic selection medium (1× Murashige and Skoog salts (pH adjusted to 5.7 with 1 M KOH), 1× Gamborg's B-5 vitamins, 0.9% phytagar (Life Technologies), and 50 mg/l kanamycin). Seeds were germinated under continuous illumination (50-75 μE/m²/second) at 22-23° C. After 7-10 days of growth under these conditions, kanamycin-resistant primary transformants (T₁ generation) were visible and obtained. These seedlings were transferred first to fresh selection plates where the seedlings continued to grow for 3-5 more days, and then to soil (Pro-Mix BX potting medium).

Primary transformants were crossed and progeny seeds (T₂) collected; kanamycin-resistant seedlings were selected and analyzed. The expression levels of the recombinant polynucleotides in the transformants varies from about a 5% expression level increase to a least a 100% expression level increase. Similar observations are made with respect to polypeptide level expression.

### Example VI

### Identification of Arabidopsis Plants with Transcription Factor Gene Knockouts

The screening of insertion mutagenized Arabidopsis collections for null mutants in a known target gene was essentially as described in Krysan et al. (1999) Plant Cell 11: 2283-2290. Briefly, gene-specific primers, nested by 5-250 base pairs to each other, were designed from the 5′ and 3′ regions of a known target gene. Similarly, nested sets of primers were also created specific to each of the T-DNA or transposon ends (the “right” and “left” borders). All possible combinations of gene specific and T-DNA/transposon primers were used to detect by PCR an insertion event within or close to the target gene. The amplified DNA fragments were then sequenced which allows the precise determination of the T-DNA/transposon insertion point relative to the target gene. Insertion events within the coding or intervening sequence of the genes were deconvoluted from a pool comprising a plurality of insertion events to a single unique mutant plant for functional characterization. The method is described in more detail in Yu and Adam, U.S. application Ser. No. 09/177,733 filed Oct. 23, 1998.

### Example VII

### Identification of Modified Phenotypes in Overexpressing Plants

Experiments were performed to identify those transformants that exhibited a morphological difference relative to wild-type control plants, i.e., a modified structure, physiology, and/or development characteristics. For such studies, the transformants were exposed to various assay conditions and novel structural, physiological responses, or developmental characteristics associated with the ectopic expression of the polynucleotides or polypeptides of the invention were observed. Examples of genes and equivalogs that confer significant improvements to overexpressing plants are noted.

Experiments were also performed to identify those transformants that exhibited an improved pathogen tolerance, with results provided in Example VIII. All four TRANSCRIPTIONAL REGULATOR OF DEFENSE RESPONSE (TDR) sequences were tested under the regulatory control of tissue-specific and inducible promoters using a two-component system. The goal of these experiments was to determine if disease resistance could be achieved while reducing detrimental pleiotropic effects of ectopic expression of the TDR genes. Three different promoters were tested in combination with all four paralogs: tomato RBCS3 (Sugita et al. (1987) Mol. Gen. Genet. 209: 247-256), Arabidopsis LTP1 (Thoma et al. (1994) Plant Physiol. 105: 35-45), and a transgenic glucocorticoid-inducible promoter (Aoyama and Chua (1997) Plant J. 11: 605-612). To test the spectrum of resistance in the two-component lines, we performed assays for Botrytis cinerea, Fusarium oxysporum, and Sclerotinia sclerotiorum. The 35S:: G1792 lines had not shown resistance to Sclerotinia in previous experiments, but this fungus was included to determine if any of the paralog genes gave enhanced resistance to a broader or different spectrum of pathogens.

For the LTP1 and RBCS3 projects, the first component (promoter::LexA/GAL4) comprised a LexA DNA binding domain fused to a GAL4 activation domain, cloned behind one of these promoters. These constructs are contained within vector backbone pMEN48 that also carried a kanamycin resistance marker, along with an opLexA::GFP reporter. The green fluorescent protein (OFP) used was EGFP, a variant available from Clontech (Palo Alto, Calif.) with enhanced signal. EGFP is soluble in the cytoplasm. Transgenic “driver lines” were first obtained containing the promoter::LexA/GAL4 component. For each promoter driver, a line was selected that showed reproducible expression of the GFP reporter gene in the desired pattern through a number of generations. A homozygous population was then established.

Having established a promoter panel, it was then possible to overexpress any transcription factor in the G1792 clade by super-transforming or crossing in a second construct (opLexA::transcription factor) carrying the transcription factor of interest cloned behind a LexA operator site. In each case this second construct carried a sulfonamide selectable marker and was contained within vector backbone.

For the preparation of dexamethasone inducible lines, a kanamycin-resistant 35S::LexA-GAL4-transactivator driver line was established and was supertransformed with opLexA::transcription factor constructs carrying a sulfonamide-resistance gene for each of the transcription factors of interest. 35S::LexA-GAL4-transactivator independent driver lines were generated at the outset of the experiment. Primary transformants were selected on kanamycin plates and screened for GFP fluorescence at the seedling stage. Any lines that showed constitutive GFP activity were discarded. At ten days, lines that showed no GFP activity were transferred onto MS agar plates containing 5 μM dexamethasone. Lines that showed strong GFP activation by two to three days following the dexamethasone treatments were marked for follow-up in the T2 generation. Following similar experiments in the T2 generation, a single line, 65, was selected for future studies. Line 65 lacked any obvious background expression and all plants showed strong GFP fluorescence following dexamethasone application. A homozygous population for line 65 was then obtained, re-checked to ensure that it still exhibited induction following dexamethasone application, and bulked. 35S::LexA-GAL4-transactivator line 65 was also crossed to an opLexA::GUS line to demonstrate that it could drive activation of targets arranged in trans.

Five T1 lines from each promoter/gene combination were selected for plate-based disease assays on the T2 generation. Included in the disease assays were challenges by one of a number of diverse fungal pathogens. T2 seeds from each line (segregating for the target transgene construct) were surface sterilized and grown on MS plates supplemented with 0.3% sucrose. Plants homozygous for each activator line and supertransformed with the target construct vector containing GUS (no transcription factor gene) were used as controls and treated in the same manner as test lines. Plants were grown in a 22° C. growth chamber under constant light for ten days. On the 10th day, seedlings were transferred to MS plates without sucrose. The dex-inducible lines were transferred to MS plates supplemented with 5 μm dexamethasone. Each plate was marked with half of the plate containing nine seedlings of an experimental line and the other half containing nine seedlings of the control line. For each experimental line, there were three test plates per pathogen plus one uninoculated plate. 35S::G1792 direct promoter/gene fusion lines were included and compared to wild-type plants as a control for the disease assays. Direct 35S/gene fusion lines were also used in the abiotic stress assay experiments, for which results are presented in Tables 5-6.

At 14 days, seedlings were inoculated by spraying the plates with a freshly prepared suspension of spores (10⁵ spores/ml, Botrytis; 10⁶ spores/ml, Fusarium) or ground, filtered hyphae (1 gm/300 ml, Sclerotinia). Plates were returned to a growth chamber with dimmed lighting on a 12 hour dark/12 hour light regimen; disease symptoms were assessed over a period of two weeks after inoculation. All lines were initially tested with Botrytis and Sclerotinia. Tolerance was quantitatively scored as the number of living plants. Numbers were plotted on a “box and whisker” diagram (FIG. 6) to determine increased survivorship of particular promoter/gene combinations. To illustrate the spread of the data, results from all lines per combination were plotted together; lines that were potentially sense-suppressed (based on disease phenotype) may skew the median towards wild type in some cases. Also, all two-component lines were segregating for the target transgene. Lines that showed tolerance to Botrytis or Sclerotinia were then tested with Fusarium. Fusarium tolerance was determined by a reduction in chlorosis and damping off symptoms.

A number of plant lines overexpressing some of the G1792 clade members were tested in a soil-based assay for resistance to powdery mildew (Erysiphe cichoracearum). Typically, eight lines per project are subjected to the Erysiphe assay. Erysiphe cichoracearum inoculum was propagated on a pad4 mutant line in the Col-0 background, which is highly susceptible to Erysiphe (Reuber et al. (1998) Plant J. 16: 473-485). Inoculum was maintained by using a small paintbrush to dust conidia from a 2-3 week old culture onto new plants (generally three weeks old). For the assay, seedlings were grown on plates for one week under 24-hour light in a germination chamber, then transplanted to soil and grown in a walk-in growth chamber under a 12-hour light/12-hour dark light regimen, 70% humidity. Each line was transplanted to two 13 cm square pots, nine plants per pot. In addition, three control plants were transplanted to each pot for direct comparison with the test line. Approximately 3.5 weeks after transplanting, plants were inoculated using settling towers, as described by Reuber et al. (1998) supra. Generally, three to four heavily infested leaves were used per pot for the disease assay. Level of fungal growth was evaluated eight to ten days after inoculation.

Assays were also performed to identify those transformants that exhibited improved abiotic stress tolerance. The germination assays in Example VIII followed modifications of the same basic protocol. Sterile seeds were sown on the conditional media listed below. Plates were incubated at 22° C. under 24-hour light (120-130 μEin/m²/s) in a growth chamber. Evaluation of germination and seedling vigor was conducted 3 to 15 days after planting. The basal media was 80% Murashige-Skoog medium (MS)+vitamins.

For stress experiments conducted with more mature plants, seeds were germinated and grown for seven days on MS+vitamins+1% sucrose at 22° C. and then transferred to cold and heat stress conditions. The plants were either exposed to cold stress (6 hour exposure to 8° C.), or heat stress (32° C. was applied for five days, after which the plants were transferred back 22° C. for recovery and evaluated after 5 days relative to controls not exposed to the depressed or elevated temperature).

The salt stress assays were intended to find genes that confer better germination, seedling vigor or growth in high salt. Evaporation from the soil surface causes upward water movement and salt accumulation in the upper soil layer where the seeds are placed. Thus, germination normally takes place at a salt concentration much higher than the mean salt concentration of the whole soil profile. Plants differ in their tolerance to NaCl depending on their stage of development, therefore seed germination, seedling vigor, and plant growth responses were evaluated.

Hyperosmotic stress assays (including NaCl and mannitol assays) were conducted to determine if an osmotic stress phenotype was NaCl-specific or if it was a general hyperosmotic stress related phenotype. Plants tolerant to hyperosmotic stress could also have more tolerance to drought and/or freezing.

For salt and hyperosmotic stress germination experiments, the medium was supplemented with 150 mM NaCl or 300 mM mannitol. Growth regulator sensitivity assays were performed in MS media, vitamins, and either 0.3 μM ABA 9.4% sucrose, or 5% glucose.

Desiccation and drought assays were performed to find genes that mediate better plant survival after short-term, severe water deprivation. Ion leakage was measured if needed.

For plate-based desiccation assays, wild-type and control seedlings were grown for 14 days on MS+Vitamins+1% Sucrose at 22° C. The plates were then left open in the sterile hood for 3 hr for hardening, and the seedlings were removed from the media and dried for 1.5 h in the sterile hood. The seedlings were transferred back to plates and incubated at 22° C. for recovery. The plants were then evaluated after another five days.

Soil-based drought screens were performed with Arabidopsis plants overexpressing the transcription factors listed in the Sequence Listing, where noted below. Seeds from wild-type Arabidopsis plants, or plants overexpressing a polypeptide of the invention, were stratified for three days at 4° C. in 0.1% agarose. Fourteen seeds of each overexpressor or wild-type were then sown in three inch clay pots containing a 50:50 mix of vermiculite:perlite topped with a small layer of MetroMix 200 and grown for fifteen days under 24 hr light. Pots containing wild-type and overexpressing seedlings were placed in flats in random order. Drought stress was initiated by placing pots on absorbent paper for seven to eight days. The seedlings were considered to be sufficiently stressed when the majority of the pots containing wild-type seedlings within a flat had become severely wilted. Pots were then re-watered and survival was scored four to seven days later. Plants were ranked against wild-type controls for each of two criteria: tolerance to the drought conditions and recovery (survival) following re-watering.

At the end of the initial drought period, each pot was assigned a numeric value score depending on the above criteria. A low value was assigned to plants with an extremely poor appearance (i.e., the plants were uniformly brown) and a high value given to plants that were rated very healthy in appearance (i.e., the plants were all green). After the plants were rewatered and incubated an additional four to seven days, the plants were reevaluated to indicate the degree of recovery from the water deprivation treatment.

An analysis was then conducted to determine which plants best survived water deprivation, identifying the transgenes that consistently conferred drought-tolerant phenotypes and their ability to recover from this treatment. The analysis was performed by comparing overall and within-flat tabulations with a set of statistical models to account for variations between batches. Several measures of survival were tabulated, including: (a) the average proportion of plants surviving relative to wild-type survival within the same flat; (b) the median proportion surviving relative to wild-type survival within the same flat; (c) the overall average survival (taken over all batches, flats, and pots); (d) the overall average survival relative to the overall wild-type survival; and (e) the average visual score of plant health before rewatering.

Sugar sensing assays were intended to find genes involved in sugar sensing by germinating seeds on high concentrations of sucrose and glucose and looking for degrees of hypocotyl elongation. The germination assay on mannitol controlled for responses related to osmotic stress. Sugars are key regulatory molecules that affect diverse processes in higher plants including germination, growth, flowering, senescence, sugar metabolism and photosynthesis. Sucrose is the major transport form of photosynthate and its flux through cells has been shown to affect gene expression and alter storage compound accumulation in seeds (source-sink relationships). Glucose-specific hexose-sensing has also been described in plants and is implicated in cell division and repression of “famine” genes (photosynthetic or glyoxylate cycles).

Temperature stress assays were carried out to find genes that confer better germination, seedling vigor or plant growth under temperature stress (cold, freezing and heat). Temperature stress cold germination experiments were carried out at 8° C. Heat stress germination experiments were conducted at 32° C. to 37° C. for 6 hours of exposure.

For nitrogen utilization assays, sterile seeds were sown onto plates containing media based on 80% MS without a nitrogen source (“low N germ” assay). For carbon/nitrogen balance (C/N) sensing assays, the media also contained 3% sucrose (−N/+G). The −“low N w/gln germ” media was identical but was supplemented with 1 mM glutamine. Plates were incubated in a 24-hour light C (120-130 μEins⁻² m⁻¹) growth chamber at 22° C. Evaluation of germination and seedling vigor was done five days after planting for C/N assays. The production of less anthocyanin on these media is generally associated with increased tolerance to nitrogen limitation, and a transgene responsible for the altered response is likely involved in the plant's ability to perceive their carbon and nitrogen status.

The transcription factor sequences of the present Sequence Listing, Tables, Figures, and their equivalogs can be used to prepare transgenic plants and plants with increased abiotic stress tolerance. The specific transgenic plants listed below are produced from sequences of the Sequence Listing, as noted. The Sequence Listing, Tables 1 and 5-40 and Examples VIII and IX provide exemplary polynucleotide and polypeptide sequences of the invention.

### Example VIII

### Genes that Confer Significant Abiotic Stress Tolerance

This example provides experimental evidence for increased abiotic stress tolerance controlled by the transcription factor polypeptides and polypeptides of the invention, indicating that sequences found within the G1792 clade of transcription factor polypeptides are functionally related and can be used to confer various types of abiotic stress tolerance in plants. As shown below, members of the G1792 clade of transcription factor polypeptides from diverse plant species, including G30, G1791, and G1792, soybean G3518 and G3520, rice G3380, G3381, G3383, G3515, and G3737, and corn G3516 and G3517 (SEQ ID NO: 7, 3, 1, 21, 25, 9, 11, 13, 15, 31, 17, and 19, respectively) increase abiotic stress tolerance when these sequences are overexpressed. From these experimental results, it may be inferred that a considerable number of sequences within the G1792 clade from diverse plant species may be used to impart increased environmental stress tolerance. A number of these genes conferred increased tolerance to multiple abiotic stresses (including disease resistance, as noted in the previous Example).

G1792 clade member overexpression also increased tolerance to growth on nitrogen-limiting conditions. As noted below, a number of transformants showed more tolerance to growth under nitrogen-limiting conditions. For example, in a root growth assay under conditions of limiting nitrogen, 35S::G1792, 35S::G3381 and 35S::G3515 lines were less stunted. In a germination assay that monitors the effect of carbon on nitrogen signaling through anthocyanin production on media with high sucrose and with or without glutamine (Hsieh et al. (1998) Proc. Natl. Acad. Sci. USA 95: 13965-13970), different lines overexpressing various clade members made less anthocyanin on high sucrose with glutamine, indicating that these sequences are likely involved in monitoring carbon and nitrogen status in plants.

Abbreviations used in Tables in this Example:

n/d=not determined
NaCl=germination assay in 150 mM NaCl
Man=germination assay in 300 mM mannitol
Suc=germination assay in 9.4% sucrose
ABA=germination assay in 0.3 μM abscisic acid
Dsc=severe desiccation assay where seedlings are dried 1.5 h, transferred to 22° C., evaluated 5 days later
Cold germ=germination at 8° C.
Cold growth=growth of plants at 8° C. until a stress response is evident
Heat germ=germination at 32° C.
Heat growth=growth of plants at 32° C. for 5 days followed by recovery at 22° C.
Low N germ=rate of germination under low nitrogen and high sucrose conditions (part of the C/N sensing assay; this germination assay monitors the effect of carbon on nitrogen signaling through anthocyanin production on media with high sucrose and with or without glutamine (Hsieh et al. (1998) Proc. Natl. Acad. Sci. USA 95: 13965-13970))
Low N root growth=degree of root development (mass, root hairs) under low nitrogen conditions
Low N w/gln germ=C/N sensing assay (Hsieh et al. (1998) Proc. Nall. Acad. Sci. USA 95: 13965-13970); this assay looks for alterations in the mechanisms plants use to sense internal levels of carbon and nitrogen metabolites which could activate signal transduction cascades that regulate the transcription of N-assimilatory genes. To determine whether these mechanisms are altered, we exploit the observation that wild-type plants grown on media containing high levels of sucrose (3%) without a nitrogen source accumulate high levels of anthocyanins. This sucrose induced anthocyanin accumulation can be relieved by the addition of either inorganic or organic nitrogen. We use glutamine as a nitrogen source since it also serves as a compound used to transport N in plants.
DPF=direct promoter fusion
TCST=two component supertransformation
++ greater enhanced tolerance compared to controls; the phenotype was very consistent and growth was significantly above the normal levels of variability observed for that assay (for ABA, much less sensitive to ABA than controls)
+ greater tolerance compared to controls; the response was consistent and was moderately above the normal levels of variability observed for that assay (for ABA, less sensitive to ABA than controls)
− less tolerance compared to controls; the response was consistent and moderately above the normal levels of variability observed for that assay (for ABA, more sensitive to ABA than controls)
   G1792 (Arabidopsis thaliana; SEQ ID NO: 1 and 2) Abiotic Stress Assay Results

Plants overexpressing G1792 under the regulatory control of the constitutive 35S promoter were generally smaller than wild-type controls, were rather dark and shiny and in some cases showed delayed flowering. 35S::G1792 lines (direct promoter fusion and two component) had better performance in a C/N sensing assay and growth under low N compared with wild-type seedlings. In addition, some direct promoter and two component lines showed tolerance to severe dehydration and cold conditions in growth assays.

G1792 overexpression also increased tolerance to growth on nitrogen-limiting conditions. 35S::G1792 transformants showed more tolerance to growth under nitrogen-limiting conditions. In the root growth assay under conditions of limiting nitrogen, 35S::G1792 lines were less stunted. In the germination assay that monitors the effect of carbon on nitrogen signaling through anthocyanin production on media with high sucrose and with or without glutamine, the 35S::G1792 lines made less anthocyanin on high sucrose with glutamine, indicating that this sequence is likely involved in monitoring carbon and nitrogen status in plants.

TABLE 5
35S::G1792 plate assay results

| | | | | | | | | | | | Nitrogen utilization assays | | |
|---|---|---|---|---|---|---|---|---|---|---|---|---|---|
| | | | | | | | Heat and cold assays | | | | Low | Low N | Low N |
| Project | | Hyperosmotic stress assays | | | | | Heat | Cold | Heat | Cold | N | w/gln | root |
| Type | Line | NaCl | Man | Suc | ABA | Dsc | germ | germ | growth | growth | germ | germ | growth |
| DPF | 301 | | | | | + | | | | | + | + | + |
| DPF | 305 | | | | | + | | + | | | + | + | ++ |
| DPF | 307 | | | | | + | | | | | | | |
| DPF | 309 | | | | | | | | | | | | |
| DPF | 310 | | | | | | | | | | | | + |
| DPF | 311 | | | + | | | | ++ | | | + | + | + |
| DPF | 312 | | | | | | | + | | | + | + | ++ |
| DPF | 313 | | | | | + | | | | | | | |
| DPF | 318 | | | | | | | | | | + | + | |
| DPF | 320 | | | | | | | | | | + | + | + |
| DPF | 5-1-5 | | | | | | | | + | | | | |
| DPF | 6 | | | | | | | | | + | + | + | + |
| DPF | 12 | | | | | | | | + | | | | |
| TCST | 402 | | | | | | | | | | + | + | + |
| TCST | 405 | | | | + | | | | | | | | |
| TCST | 407 | | | | | | | | | | + | + | + |
| TCST | 413 | | | | + | | | | | | | | + |
| TCST | 417 | | | | | | | | | + | + | + | + |
| TCST | 419 | | | | | + | | | | + | + | + | |
| TCST | 420 | | | | | | | | | + | + | + | + |
| TCST | 521 | | | | | | | | | + | + | + | + |
| TCST | 523 | | | | | + | | | | | + | + | + |
| TCST | 525 | | | | | | | | | | | + | + |
| TCST | 526 | | | | | | | | | | | + | + |
| TCST | 528 | | | | | + | | | | | + | + | + |
| TCST | 531 | | | | | + | | | | + | + | + | |
| TCST | 532 | | | | | | | | | + | + | + | + |
| TCST | 533 | | | | | + | ++ | + | | | + | + | + |

35S::G1792 lines exhibited markedly enhanced drought tolerance compared to wild-type, both in terms of their appearance at the end of the drought period, and in survivability following re-watering. Plant lines reported in more than one row indicate duplicate assays. Asterisks indicate statistically significant performance of experimental lines over controls (lines performed better than control; significant at P<0.11).

TABLE 6
Performance of 35S::G1792 (Arabidopsis) lines in soil-based drought assays

| | | | | | Evaluation after rewatering | | |
|---|---|---|---|---|---|---|---|
| | | Evaluation after drought treatment | | | Mean survival | Mean | |
| | | Mean score, | Mean | P value | for | survival | P value for |
| | Project | experimental | score, | score, | experimental | for | difference in |
| Line | Type | line | control | difference | line | control | survival |
| 523 | TCST | 1.7 | 0.90 | 0.050* | 0.41 | 0.16 | 0.000012* |
| 528 | TCST | 0.41 | 0.16 | 0.000012* | 0.41 | 0.16 | 0.000012* |
| 5 | DPF | 0.41 | 0.16 | 0.000012* | 0.41 | 0.24 | 0.051* |
| 5 | DPF | 2.6 | 1.3 | 0.011* | 0.30 | 0.21 | 0.033* |
| 6 | DPF | 4.7 | 1.7 | 0.00087* | 0.49 | 0.24 | 0.0000097* |
| 6 | DPF | 1.7 | 1.3 | 0.41 | 0.26 | 0.21 | 0.22 |
| 301 | DPF | 1.5 | 0.78 | 0.32 | 0.15 | 0.079 | 0.092* |
| 311 | DPF | 1.3 | 1.5 | 0.80 | 0.29 | 0.19 | 0.068* |

The majority of the Arabidopsis lines overexpressing G 1792 under the regulatory control of the SUC2 promoter were similar to controls in their development and morphology. Most lines performed better than wild-type controls in at least one plate-based physiological and/or nitrogen utilization assay.

TABLE 7
SUC2::G1792 plate assay results

| | | | | | | | | | | | Nitrogen utilization assays | | |
|---|---|---|---|---|---|---|---|---|---|---|---|---|---|
| | | | | | | | Heat and cold assays | | | | Low | Low N | Low N |
| Project | | Hyperosmotic stress assays | | | | | Heat | Cold | Heat | Cold | N | w/gln | root |
| Type | Line | NaCl | Man | Suc | ABA | Dsc | germ | germ | growth | growth | germ | germ | growth |
| TCST | 821 | | | | | | | | | | | + | n/d |
| TCST | 822 | | | | | | | | | | | | n/d |
| TCST | 823 | | | | | + | | + | | | + | + | n/d |
| TCST | 824 | | | | + | | | | | | | | n/d |
| TCST | 825 | | | | | + | | | | | | + | n/d |
| TCST | 826 | | | | | + | | | | | | + | n/d |
| TCST | 827 | | | | | | | + | | | + | + | n/d |
| TCST | 828 | | | | + | + | | | | | | | n/d |
| TCST | 829 | | | + | | + | | + | | | | + | n/d |
| TCST | 830 | | | | | + | | + | | | | | n/d |

The majority of the Arabidopsis lines overexpressing G1792 under the regulatory control of the RBCS3 promoter were slightly smaller, darker green, and later developing than controls, but these phenotypes were much less severe than those of 35S::G1792 plants. Three out of ten lines showed enhanced tolerance to sodium chloride in a germination assay.

TABLE 8
RBCS3::G1792 plate assay results

| | | | | | | | | | | | Nitrogen utilization assays | | |
|---|---|---|---|---|---|---|---|---|---|---|---|---|---|
| | | | | | | | Heat and cold assays | | | | Low | Low N | Low N |
| Project | | Hyperosmotic stress assays | | | | | Heat | Cold | Heat | Cold | N | w/gln | root |
| Type | Line | NaCl | Man | Suc | ABA | Dsc | germ | germ | growth | growth | germ | germ | growth |
| TCST | 362 | | | | | | | | | | | | |
| TCST | 366 | + | | + | | | | | | | | | |
| TCST | 367 | | | | | | | | | | | | |
| TCST | 368 | | | | | | | | | + | | | |
| TCST | 369 | + | | | | | | | | | | | |
| TCST | 370 | | | | | | | | | + | | | |
| TCST | 372 | | | | | | | | | | | | |
| TCST | 374 | + | | | | | | | | | | | |
| TCST | 378 | | + | | | + | | | | | | | |
| TCST | 379 | | | | | | | | | | | | |

Some epidermal-specific LTP1::G1792 T1 lines flowered slightly early, but otherwise LTP1::G1792 plants were not consistently different from controls. LTP1::G1792 lines showed a better performance than wild-type controls in a low N growth assay on plates.

TABLE 9
LTP1::G1792 plate assay results

| | | | | | | | | | | | Nitrogen utilization assays | | |
|---|---|---|---|---|---|---|---|---|---|---|---|---|---|
| | | | | | | | Heat and cold assays | | | | Low | Low N | Low N |
| Project | | Hyperosmotic stress assays | | | | | Heat | Cold | Heat | Cold | N | w/gln | root |
| Type | Line | NaCl | Man | Suc | ABA | Dsc | germ | germ | growth | growth | germ | germ | growth |
| TCST | 341 | | | | | | | | | | | | |
| TCST | 342 | | | | | | | | | | | | + |
| TCST | 346 | | | + | | | | | | | | | |
| TCST | 347 | | | | | | | | | | | | |
| TCST | 348 | | | | | | | | | | | | |
| TCST | 350 | | | + | | | | | | | | | + |
| TCST | 352 | | | | | | | | | | | | |
| TCST | 353 | | | | | | | | | | | | + |
| TCST | 354 | | | | | | | | | | | | + |
| TCST | 357 | | | | | | | | | | | | |

A number of Arabidopsis lines overexpressing GI 792 under the regulatory control of the STM (shoot apical meristem-specific) promoter were smaller than wild-type controls. Other lines showed no consistent developmental or morphological differences with respect to the controls. Three lines were less sensitive to ABA, and three lines were more tolerant to germination under cold conditions than wild-type controls.

TABLE 10
STM::G1792 plate assay results

| | | | | | | | | | | | Nitrogen utilization assays | | |
|---|---|---|---|---|---|---|---|---|---|---|---|---|---|
| | | | | | | | Heat and cold assays | | | | Low | Low N | Low N |
| Project | | Hyperosmotic stress assays | | | | | Heat | Cold | Heat | Cold | N | w/gln | root |
| Type | Line | NaCl | Man | Suc | ABA | Dsc | germ | germ | growth | growth | germ | germ | growth |
| TCST | 112 | n/d | + | | n/d | | n/d | | n/d | | | | |
| TCST | 112 | n/d | + | | n/d | | n/d | | n/d | | | | |
| TCST | 112 | n/d | | | n/d | | n/d | + | n/d | | | | |
| TCST | 112 | n/d | + | | n/d | | n/d | + | n/d | | | | |
| TCST | 112 | n/d | | | n/d | | n/d | + | n/d | | | | |
| TCST | 114 | n/d | | | n/d | | n/d | | n/d | | | | |
| TCST | 114 | n/d | | | n/d | | n/d | | n/d | | | | |
| TCST | 114 | n/d | | | n/d | | n/d | | n/d | | | | |
| TCST | 114 | n/d | | | n/d | | n/d | | n/d | | | | |
| TCST | 114 | n/d | | | n/d | | n/d | | n/d | | | | |

A number of Arabidopsis lines overexpressing G1792 under the regulatory control of the RD29A (stress-inducible) promoter were smaller than wild-type controls. Thus far, some of the lines tested were less sensitive to ABA and more tolerant to salt than wild-type controls, and had more root growth in low nitrogen conditions.

TABLE 11
RD29A::G1792 plate assay results

| | | | | | | | | | | | Nitrogen utilization assays | | |
|---|---|---|---|---|---|---|---|---|---|---|---|---|---|
| | | | | | | | Heat and cold assays | | | | Low | Low N | Low N |
| Project | | Hyperosmotic stress assays | | | | | Heat | Cold | Heat | Cold | N | w/gln | root |
| Type | Line | NaCl | Man | Suc | ABA | Dsc | germ | germ | growth | growth | germ | germ | growth |
| TCST | 501 | | | | | | | | | | | | + |
| TCST | 502 | | | | + | | | | | | | | + |
| TCST | 503 | | | | | | | | | | | | |
| TCST | 504 | + | | | + | | | | | | | | |
| TCST | 505 | + | | | | | | | | | | | |
| TCST | 506 | + | | | + | | | | | | | | |
| TCST | 507 | | | | + | | | | | | | | + |
| TCST | 701 | | | | | + | | | | | | | |
| TCST | 702 | + | | | | + | | | | | | | + |
| TCST | 703 | | | | | | | | | | | | + |
| TCST | 704 | + | | | + | | | | | | | | |

A number of Arabidopsis lines overexpressing G1792 under the regulatory control of the RSI1 (Root-tissue-specific) promoter were similar in morphology and development to wild-type controls, including some of the lines that were positive in a C/N sensing assay.

TABLE 12
RSI1::G1792 plate assay results

| | | | | | | | | | | | Nitrogen utilization assays | | |
|---|---|---|---|---|---|---|---|---|---|---|---|---|---|
| | | | | | | | Heat and cold assays | | | | Low | Low N | Low N |
| Project | | Hyperosmotic stress assays | | | | | Heat | Cold | Heat | Cold | N | w/gln | root |
| Type | Line | NaCl | Man | Suc | ABA | Dsc | germ | germ | growth | growth | germ | germ | growth |
| TCST | 1321 | n/d | n/d | | n/d | | n/d | | | | | + | |
| TCST | 1322 | n/d | n/d | | n/d | | n/d | | | | | + | |
| TCST | 1323 | n/d | n/d | | n/d | | n/d | | | | | + | |
| TCST | 1324 | n/d | n/d | | n/d | | n/d | | | | | + | |
| TCST | 1325 | n/d | n/d | | n/d | | n/d | | | | | | |
| TCST | 1326 | n/d | n/d | | n/d | | n/d | | | | | | |
| TCST | 1327 | n/d | n/d | | n/d | | n/d | | | | | | + |
| TCST | 1328 | n/d | n/d | | n/d | | n/d | | | | | | |
| TCST | 1329 | n/d | n/d | | n/d | | n/d | | | | | | + |
| TCST | 1330 | n/d | n/d | | n/d | | n/d | | | | | | |

N-GAL4-TA G1792 plants exhibited comparable phenotypes to 35S::G1792 lines and all (to varying extents) were dwarfed, late flowering, dark in coloration, and had a shiny appearance. These plants showed a better performance than controls in severe dehydration and cold germination assays performed on plates. Three lines also showed a better performance than controls in a plate based low N growth assay. The phenotype seen was less potent than with overexpression lines for the native form of G1792, suggesting that the GAL4-G1792 fusion might have a reduction in activity relative to the native form.

TABLE 13
Superactivated N-GAL4-TA G1792 plate assay results

| | | | | | | | | | | | Nitrogen utilization assays | | |
|---|---|---|---|---|---|---|---|---|---|---|---|---|---|
| | | | | | | | Heat and cold assays | | | | Low | Low N | Low N |
| Project | | Hyperosmotic stress assays | | | | | Heat | Cold | Heat | Cold | N | w/gln | root |
| Type | Line | NaCl | Man | Suc | ABA | Dsc | germ | germ | growth | growth | germ | germ | growth |
| GAL4 | 645 | | | | | | | | | | | | − |
| N-terminal | | | | | | | | | | | | | |
| fusion | | | | | | | | | | | | | |
| GAL4 | 646 | | | | | + | | + | | | | | |
| N-terminal | | | | | | | | | | | | | |
| fusion | | | | | | | | | | | | | |
| GAL4 | 661 | | | | | | | + | | | | | |
| N-terminal | | | | | | | | | | | | | |
| fusion | | | | | | | | | | | | | |
| GAL4 | 662 | | | | | + | | | | | | | |
| N-terminal | | | | | | | | | | | | | |
| fusion | | | | | | | | | | | | | |
| GAL4 | 663 | | | | | | | | | | | | |
| N-terminal | | | | | | | | | | | | | |
| fusion | | | | | | | | | | | | | |
| GAL4 | 664 | | | | | | | + | | | | | + |
| N-terminal | | | | | | | | | | | | | |
| fusion | | | | | | | | | | | | | |
| GAL4 | 665 | | | | | | | | | | | | |
| N-terminal | | | | | | | | | | | | | |
| fusion | | | | | | | | | | | | | |
| GAL4 | 666 | | | | | + | | | | | | | + |
| N-terminal | | | | | | | | | | | | | |
| fusion | | | | | | | | | | | | | |
| GAL4 | 667 | | | | | + | | + | | | | | + |
| N-terminal | | | | | | | | | | | | | |
| fusion | | | | | | | | | | | | | |
| GAL4 | 668 | | | | | | | + | + | | | | |
| N-terminal | | | | | | | | | | | | | |
| fusion | | | | | | | | | | | | | |

G1792 (and related genes) also respond in baseline microarray experiments. G1792 and related genes have been identified as responding to abiotic stresses in microarray experiments in which wild-type Columbia plants were been treated with various abiotic stresses. G1792 transcript in roots peaks four hours after mannitol treatment, reaching an expression level approximately 24-fold higher than mock treated plants. G1792 transcript levels in roots in NaCl treated plants reach levels eight-fold higher than mock treated plants at eight hours. Interestingly, G1792 expression is down-regulated in both soil-based drought experiments and upon treatment with ABA. Expression levels in both cases are down-regulated approximately three-fold.

G30 (Arabidopsis thaliana; SEQ ID NO: 7 and 8) Abiotic Stress Assay Results

Plants overexpressing G30 under the regulatory control of the epidermal-specific LTP1 were small in size and dark in color, with curling upright leaves compared to controls. All lines also flowered and developed late. The small, dark green, and late flowering phenotypes are typical of members of the G1792 clade, though much less severe than seen in 35S::G30 plants.

Three out of ten LTP1::G30 lines showed better performance in a growth assay on low nitrogen compared with wild-type control seedlings. Three other lines did not accumulate anthocyanins in a cold germination assay, indicating that these lines may be more tolerant to cold germination.

TABLE 14
LTP1::G30 plate assay results

| | | | | | | | | | | | Nitrogen utilization assays | | |
|---|---|---|---|---|---|---|---|---|---|---|---|---|---|
| | | | | | | | Heat and cold assays | | | | Low | Low N | Low N |
| Project | | Hyperosmotic stress assays | | | | | Heat | Cold | Heat | Cold | N | w/gln | root |
| Type | Line | NaCl | Man | Suc | ABA | Dsc | germ | germ | growth | growth | germ | germ | growth |
| TCST | 341 | | | | | | | | | | | | |
| TCST | 342 | | | | | | | | | | | | + |
| TCST | 344 | | | | | | | | | | | | + |
| TCST | 345 | | | | | | | | | | | | + |
| TCST | 346 | | | | | | | | | | | | |
| TCST | 381 | | | | | + | | | | | | | |
| TCST | 382 | | | | | | | + | | | | | |
| TCST | 384 | | | | | | | + | | | | | |
| TCST | 385 | | | | | | | | | | | | |
| TCST | 387 | | | | | | | + | | | | | |

Most of the Arabidopsis lines overexpressing G30 under the regulatory control of the RD29A promoter (line 5; stress inducible) were similar to wild-type controls in their development and morphology. This promoter-gene combination conferred greater tolerance to salt, ABA, germination in cold and low nitrogen conditions than the controls.

TABLE 15
RD29A - line 5::G30 plate assay results

| | | | | | | | | | | | Nitrogen utilization assays | | |
|---|---|---|---|---|---|---|---|---|---|---|---|---|---|
| | | | | | | | Heat and cold assays | | | | Low | Low N | Low N |
| Project | | Hyperosmotic stress assays | | | | | Heat | Cold | Heat | Cold | N | w/gln | root |
| Type | Line | NaCl | Man | Suc | ABA | Dsc | germ | germ | growth | growth | germ | germ | growth |
| TCST | 521 | + | | | + | | | + | | | | | |
| TCST | 522 | + | | | | | | + | | | | | |
| TCST | 581 | | | | | | | | | | | | |
| TCST | 582 | | | | | | | | | | | | |
| TCST | 583 | + | | | | | | + | | + | | | |
| TCST | 603 | + | | | + | | | | | | | | |
| TCST | 604 | | | | + | | | | | | | | |
| TCST | 661 | + | | | + | | | | | | | | |
| TCST | 662 | + | | | | | | | | | | | |
| TCST | 663 | + | | | + | | | | | | | | + |
| TCST | 664 | + | | | | | | | | | | | |
| TCST | 665 | | | | | | | | | | | | + |
| TCST | 666 | | | | | | | | | | | | |

Most Arabidopsis lines overexpressing G30 under the regulatory control of the SUC2 promoter (vascular-specific) were dark, shiny, and small. However, this promoter-gene combination conferred greater tolerance to mannitol, sucrose, desiccation, and germination in cold than wild-type controls. The overexpressors also performed better than controls in low nitrogen and nitrogen utilization assays.

TABLE 16
SUC2::G30 plate assay results

| | | | | | | | | | | | Nitrogen utilization assays | | |
|---|---|---|---|---|---|---|---|---|---|---|---|---|---|
| | | | | | | | Heat and cold assays | | | | Low | Low N | Low N |
| Project | | Hyperosmotic stress assays | | | | | Heat | Cold | Heat | Cold | N | w/gln | root |
| Type | Line | NaCl | Man | Suc | ABA | Dsc | germ | germ | growth | growth | germ | germ | growth |
| TCST | 548 | | | | | | | + | | | + | + | n/d |
| TCST | 549 | | | | | | | + | | | | | n/d |
| TCST | 550 | | + | + | | | | + | + | | | + | n/d |
| TCST | 551 | | + | + | | | | + | | | | + | n/d |
| TCST | 552 | | + | | | + | | | | | | + | n/d |
| TCST | 554 | | | | | | | + | | | | | n/d |
| TCST | 557 | | | | | + | | | | | | + | n/d |
| TCST | 558 | | | | | | | | | | | + | n/d |
| TCST | 559 | | + | | | + | | + | | | | + | n/d |
| TCST | 560 | | + | | | | | + | | | + | + | n/d |

Many of the Arabidopsis lines overexpressing G30 under the regulatory control of the RSI1 promoter (root-specific) were small with dark green, shiny, and upright leaves. At least one line was indistinguishable from controls at all stages of growth, except for being more tolerant to cold during germination.

TABLE 17
SUC2::G30 plate assay results

| | | | | | | | | | | | Nitrogen utilization assays | | |
|---|---|---|---|---|---|---|---|---|---|---|---|---|---|
| | | | | | | | Heat and cold assays | | | | Low | Low N | Low N |
| Project | | Hyperosmotic stress assays | | | | | Heat | Cold | Heat | Cold | N | w/gln | root |
| Type | Line | NaCl | Man | Suc | ABA | Dsc | germ | germ | growth | growth | germ | germ | growth |
| TCST | 781 | n/d | n/d | | n/d | | n/d | + | n/d | n/d | n/d | n/d | n/d |
| TCST | 782 | n/d | n/d | | n/d | | n/d | + | n/d | n/d | n/d | n/d | n/d |
| TCST | 783 | n/d | n/d | | n/d | | n/d | + | n/d | n/d | n/d | n/d | n/d |
| TCST | 784 | n/d | n/d | | n/d | | n/d | + | n/d | n/d | n/d | n/d | n/d |
| TCST | 785 | n/d | n/d | | n/d | | n/d | + | n/d | n/d | n/d | n/d | n/d |
| TCST | 786 | n/d | n/d | | n/d | + | n/d | + | n/d | n/d | n/d | n/d | n/d |
| TCST | 787 | n/d | n/d | | n/d | | n/d | | n/d | n/d | n/d | n/d | n/d |
| TCST | 788 | n/d | n/d | | n/d | | n/d | | n/d | n/d | n/d | n/d | n/d |
| TCST | 789 | n/d | n/d | | n/d | | n/d | | n/d | n/d | n/d | n/d | n/d |
| TCST | 790 | n/d | n/d | | n/d | | n/d | | n/d | .n/d | n/d | n/d | n/d |

G1791 (Arabidopsis thaliana; SEQ ID NO: 3 and 4) Abiotic Stress Assay Results

In general, two-component G1791 lines under the regulatory control of the leaf-specific RBCS3 promoter (RBCS3::G1791) were small compared to controls. Several lines were slightly late flowering. The lines were tested in plate based assays and showed a better performance than controls in ABA germination and cold growth assays.

TABLE 18
RBCS3::G1791 plate assay results

| | | | | | | | | | | | Nitrogen utilization assays | | |
|---|---|---|---|---|---|---|---|---|---|---|---|---|---|
| | | | | | | | Heat and cold assays | | | | Low | Low N | Low N |
| Project | | Hyperosmotic stress assays | | | | | Heat | Cold | Heat | Cold | N | w/gln | root |
| Type | Line | NaCl | Man | Suc | ABA | Dsc | germ | germ | growth | growth | germ | germ | growth |
| TCST | | | | | | | | | | | | | |
| TCST | | | | | + | | | | | | | | |
| TCST | | | | | + | | | | | | | | |
| TCST | | | | | | | | | | | | | |
| TCST | | | | | + | | | | | + | | | |
| TCST | | | | | + | | | | | + | | | |
| TCST | | | | | | | | | | | | | |
| TCST | | | | | | | | | | | | | |
| TCST | | | | | + | | | | | + | | | |
| TCST | | | | | | | | | | | | | |

Some of the Arabidopsis lines overexpressing G1791 under the regulatory control of the stress inducible RD29A promoter were small and late developing. Other lines were similar to wild-type controls in their development and morphology. This promoter-gene combination conferred greater tolerance to salt, ABA, and low nitrogen conditions than the controls.

TABLE 19
RD29A::G1791 plate assay results

| | | | | | | | | | | | Nitrogen utilization assays | | |
|---|---|---|---|---|---|---|---|---|---|---|---|---|---|
| | | | | | | | Heat and cold assays | | | | Low | Low N | Low N |
| Project | | Hyperosmotic stress assays | | | | | Heat | Cold | Heat | Cold | N | w/gln | root |
| Type | Line | NaCl | Man | Suc | ABA | Dsc | germ | germ | growth | growth | germ | germ | growth |
| TCST | 561 | | | | | | | | | | | | |
| TCST | 563 | | | | | | | | | | | | + |
| TCST | 564 | | | | | | | | | | | | |
| TCST | 681 | | | | | | | | | | | | + |
| TCST | 682 | | | | | + | | | | | | | |
| TCST | 683 | | | | | | | + | | | | | |
| TCST | 684 | | | | | | | | | | | | |
| TCST | 685 | | | | | | | | | | | | + |
| TCST | 686 | + | | | + | | | | | | | | |
| TCST | 687 | + | | | + | | | | | | | | |

G1795 (Arabidopsis thaliana; SEQ ID NO: 5 and 6) Abiotic Stress Assay Results

In general, two-component G1792 lines under the regulatory control of the vascular-specific SUC2 promoter (RBCS3::G1791) were small, dark green, with shiny, curly leaves compared to controls. Several lines were in their development. The lines were tested in plate based assays and showed a better performance than controls in mannitol, ABA, desiccation and root growth on low nitrogen assays.

TABLE 20
SUC2::G1795 plate assay results

| | | | | | | | | | | | Nitrogen utilization assays | | |
|---|---|---|---|---|---|---|---|---|---|---|---|---|---|
| | | | | | | | Heat and cold assays | | | | Low | Low N | Low N |
| Project | | Hyperosmotic stress assays | | | | | Heat | Cold | Heat | Cold | N | w/gln | root |
| Type | Line | NaCl | Man | Suc | ABA | Dsc | germ | germ | growth | growth | germ | germ | growth |
| TCST | 481 | | + | | + | + | | | | | | | |
| TCST | 482 | | + | | + | | | | | | | | + |
| TCST | 483 | | + | | + | | | | | | | | + |
| TCST | 484 | | + | | + | + | | | | | | | |
| TCST | 485 | | + | | + | | | | | | | | + |
| TCST | 486 | | | | | | | | | + | | | |
| TCST | 487 | | | | | | | | | | | | |
| TCST | 488 | | | | | + | | | | | | | |
| TCST | 489 | | + | | | + | | | | | | | |
| TCST | 490 | | | | | | | | | | | | |

One SUC2::G1795 line exhibited better drought tolerance than wild-type controls in survivability following re-watering. Asterisks indicate statistically significant performance of experimental lines over controls (lines performed better than control; significant at P<0.11).

TABLE 21
Performance of SUC2::G1795 (Oryza sativa) lines in soil-based drought assays

| | | | | | Evaluation after rewatering | | |
|---|---|---|---|---|---|---|---|
| | | Evaluation after drought treatment | | | Mean survival | Mean | |
| | | Mean score, | Mean | P value | for | survival | P value for |
| | Project | experimental | score, | score, | experimental | for | difference in |
| Line | Type | line | control | difference | line | control | survival |
| 481 | TCST | 1.6 | 1.1 | 0.13 | 0.32 | 0.21 | 0.031* |
| 481 | TCST | 1.5 | 1.4 | 0.69 | 0.29 | 0.24 | 0.34 |

G1266 (Arabidopsis thaliana; SEQ ID NO: 37 and 38) Abiotic Stress Assay Results

G1266 is an Arabidopsis sequence related to G1792 (FIG. 5). Many of the 35S::G1266 lines were small and spindly. Five out often 35S::G1266 (direct promoter fusion) lines were insensitive to ABA in a germination assay. Two of these lines were also tolerant to NaCl and mannitol in a germination assay. Two other lines were more tolerant to cold in another germination assay.

TABLE 22
35S::G1266 plate assay results

| | | | | | | | | | | Nitrogen utilization assays | | |
|---|---|---|---|---|---|---|---|---|---|---|---|---|
| | | | | | | Heat and cold assays | | | | Low | Low N | Low N |
| | Hyperosmotic stress assays | | | | | Heat | Cold | Heat | Cold | N | w/gln | root |
| Line | NaCl | Man | Suc | ABA | Dsc | germ | germ | growth | growth | germ | germ | growth |
| 304 | | | | | | | | | | | | |
| 307 | | | | | | | | | | | | |
| 308 | + | + | | + | | | | | | | | |
| 309 | + | + | | + | | | | | | | | |
| 311 | | | | | | | | | | | | |
| 312 | | | | + | | | + | | | | | |
| 313 | | | | + | | | + | | | | | |
| 315 | | | | + | | | | | | | | |
| 316 | | | | | | | | | | | | |
| 320 | | | | | | | | | | | | |

G1752 (Arabidopsis thaliana; SEQ ID NO: 41 and 42) Abiotic Stress Assay Results

G1752 is an Arabidopsis sequence related to G1792 (FIG. 5). Three out of seven 35S::G1752 (direct promoter fusion) lines were tolerant to mannitol in a germination assay. These three lines were a darker green than control seedlings, but appeared somewhat smaller. Several lines were small, chlorotic, and had less root growth than wild-type controls.

TABLE 23
35S::G1752 plate assay results

| | | | | | | | | | | Nitrogen utilization assays | | |
|---|---|---|---|---|---|---|---|---|---|---|---|---|
| | | | | | | Heat and cold assays | | | | Low | Low N | Low N |
| | Hyperosmotic stress assays | | | | | Heat | Cold | Heat | Cold | N | w/gln | root |
| Line | NaCl | Man | Suc | ABA | Dsc | germ | germ | growth | growth | germ | germ | growth |
| 304 | | | n/d | | | | | n/d | + | + | + | |
| 305 | | + | n/d | | | | | n/d | + | | | |
| 319 | | + | | | | + | | n/d | | + | + | |
| 323 | | + | n/d | | | | | | | | | |
| 324 | | | n/d | | | | | | | | | |
| 331 | | | n/d | | | | | | | | | |
| 337 | | | n/d | | | | | + | | + | + | |

G3380 (Oryza sativa; SEQ ID NO: 9 and 10) Abiotic Stress Assay Results

35S::G3380 overexpressors were generally small in size. Six of ten 35S::G3380 (direct promoter fusion) lines were less sensitive to ABA than wild-type controls. Five of ten lines performed better than wild-type seedlings in the mannitol germination assay. Two lines also did well when germinated in the presence of sucrose. Some lines also showed tolerance to NaCl, desiccation, germination and growth in heat, and growth in cold conditions.

TABLE 24
35S::G3380 plate assay results

| | | | | | | | | | | Nitrogen utilization assays | | |
|---|---|---|---|---|---|---|---|---|---|---|---|---|
| | | | | | | Heat and cold assays | | | | Low | Low N | Low N |
| | Hyperosmotic stress assays | | | | | Heat | Cold | Heat | Cold | N | w/gln | root |
| Line | NaCl | Man | Suc | ABA | Dsc | germ | germ | growth | growth | germ | germ | growth |
| 301 | | + | + | + | + | | | | | | | |
| 302 | | | | | + | | | | | | | |
| 304 | | | | | | | | | + | | | |
| 305 | | | + | | | | | | + | | | |
| 306 | | | | + | | | | | + | | | |
| 307 | + | + | | + | | + | | | | | | |
| 308 | | | | | | | | | | | | |
| 309 | | + | | + | | | | + | | | | |
| 321 | | + | | + | | | | | | | | |
| 322 | | + | | + | | + | | | | | | |

Three 35S::G3380 lines were more drought tolerance than wild type, both in terms of appearance at the end of the drought period, and in survivability following re-watering. Asterisks indicate statistically significant performance of experimental lines over controls (lines performed better than control; significant at P<0.11).

TABLE 25
Performance of 35S::G3380 (Oryza sativa) lines in soil-based drought assays

| | | | | | Evaluation after rewatering | | |
|---|---|---|---|---|---|---|---|
| | | Evaluation after drought treatment | | | Mean survival | Mean | |
| | | Mean score, | Mean | P value | for | survival | P value for |
| | Project | experimental | score, | score, | experimental | for | difference in |
| Line | Type | line | control | difference | line | control | survival |
| 301 | DPF | 2.3 | 1.4 | 0.023* | 0.41 | 0.40 | 0.90 |
| 301 | DPF | 1.5 | 1.3 | 0.59 | 0.27 | 0.22 | 0.37 |
| 307 | DPF | 3.0 | 2.0 | 0.12 | 0.54 | 0.42 | 0.043* |
| 307 | DPF | 1.9 | 1.0 | 0.00053* | 0.37 | 0.20 | 0.0022* |
| 322 | DPF | 3.0 | 1.5 | 0.00086* | 0.65 | 0.33 | 0.00000013* |
| 322 | DPF | 2.8 | 1.1 | 0.0015* | 0.57 | 0.29 | 0.0000027* |

G3381 (Oryza sativa; SEQ ID NO: 11 and 12) Abiotic Stress Assay Results

35S::G3381 lines were generally small and dark green. Three out of four 35S::G3381 (direct promoter fusion) lines were more tolerant than wild-type seedlings in a germination assay under cold conditions and two of these lines were more tolerant to mannitol. Some lines were also more tolerant to NaCl, ABA, and heat.

TABLE 26
35S::G3381 plate assay results

| | | | | | | | | | | Nitrogen utilization assays | | |
|---|---|---|---|---|---|---|---|---|---|---|---|---|
| | | | | | | Heat and cold assays | | | | Low | Low N | Low N |
| | Hyperosmotic stress assays | | | | | Heat | Cold | Heat | Cold | N | w/gln | root |
| Line | NaCl | Man | Suc | ABA | Dsc | germ | germ | growth | growth | germ | germ | growth |
| 301 | | | | | | | | | | | | |
| 302 | | + | | + | | | ++ | | | | | |
| 304 | + | + | | | | | + | | | | | |
| 306 | | | | | | + | + | | | + | + | + |

One 35S::G3381 line exhibited more drought tolerance than wild type, both in terms of appearance at the end of the drought period, and in survivability following re-watering. Asterisks indicate statistically significant performance of experimental lines over controls (lines performed better than control; significant at P<0.11).

TABLE 27
Performance of 35S::G3381 (Oryza sativa) lines in soil-based drought assays

| | | | | | Evaluation after rewatering | | |
|---|---|---|---|---|---|---|---|
| | | Evaluation after drought treatment | | | Mean survival | Mean | |
| | | Mean score, | Mean | P value | for | survival | P value for |
| | Project | experimental | score, | score, | experimental | for | difference in |
| Line | Type | line | control | difference | line | control | survival |
| 302 | DPF | 4.5 | 2.7 | 0.0049* | 0.67 | 0.42 | 0.00050* |

G3383 (Oryza sativa; SEQ ID NO: 13 and 14) Abiotic Stress Assay Results

35S::G3383 (direct promoter fusion) lines have been analyzed in abiotic stress assays. Seven out of ten lines showed tolerance to cold temperatures in a growth assay. Four of these lines were also tolerant to mannitol in a germination assay. Three of the seven lines also performed better than wild-type control seedlings in a severe dehydration assay.

TABLE 28
35S::G3383 plate assay results

| | | | | | | | | | | Nitrogen utilization assays | | |
|---|---|---|---|---|---|---|---|---|---|---|---|---|
| | | | | | | Heat and cold assays | | | | Low | Low N | Low N |
| | Hyperosmotic stress assays | | | | | Heat | Cold | Heat | Cold | N | w/gln | root |
| Line | NaCl | Man | Suc | ABA | Dsc | germ | germ | growth | growth | germ | germ | growth |
| 305 | | | | | | | | | | | | |
| 306 | | | | | | | | | | | | |
| 308 | | | | | + | | | | + | | | |
| 310 | | | | | | | | | | | | |
| 311 | | + | | | | | + | | + | | | + |
| 312 | | | | | + | | | | + | | | − |
| 313 | | + | | | + | | + | | | | | |
| 314 | | + | | | | | | | + | | | |
| 316 | | + | | | | | | | + | | | |
| 317 | | | | | | | + | | + | | | + |

G3515 (Oryza saliva; SEQ ID NO: 15 and 16) Abiotic Stress Assay Results

35S::G3515 (direct promoter fusion) lines were small relative to controls until in later stages of development. These lines were analyzed in abiotic stress assays. Five out of ten lines showed tolerance to salt in a germination assay.

TABLE 29
35S::G3515 plate assay results

| | | | | | | | | | | Nitrogen utilization assays | | |
|---|---|---|---|---|---|---|---|---|---|---|---|---|
| | | | | | | Heat and cold assays | | | | Low | Low N | Low N |
| | Hyperosmotic stress assays | | | | | Heat | Cold | Heat | Cold | N | w/gln | root |
| Line | NaCl | Man | Suc | ABA | Dsc | germ | germ | growth | growth | germ | germ | growth |
| 304 | | | | | | | | | | | | + |
| 306 | | | | | | | | | | | | ++ |
| 308 | | | | | | | | | | | | |
| 309 | | | | | | | | | | | | |
| 310 | | | | | | | | | + | | | |
| 313 | | + | | | | | | | + | | | |
| 314 | | + | | | | | | | | | | |
| 315 | | | | | | | | | | | | |
| 319 | | | | | | | + | + | | | | |
| 320 | | | | | | | | | | | | |

Three 35S::G3515 lines were more drought tolerant than wild type, both in terms of appearance at the end of the drought period, and in survivability following re-watering. Asterisks indicate statistically significant performance of experimental lines over controls (lines performed better than control; significant at P<0.11).

TABLE 30
Performance of 35S::G3515 (Oryza sativa) lines in soil-based drought assays

| | | | | | Evaluation after rewatering | | |
|---|---|---|---|---|---|---|---|
| | Evaluation after drought treatment | | | | Mean survival | Mean | |
| | | Mean score, | Mean | P value | for | survival | P value for |
| | Project | experimental | score, | score, | experimental | for | difference in |
| Line | Type | line | control | difference | line | control | survival |
| 310 | DPF | 0.67 | 0.33 | 0.45 | 0.19 | 0.032 | 0.00067* |
| 313 | DPF | 1.0 | 0.33 | 0.18 | .27 | 0.032 | 0.000015* |
| 319 | DPF | 1.5 | 0.33 | 0.039* | 0.35 | 0.032 | 0.00000063* |

G3516 (Zea mays; SEQ ID NO: 17 and 18) Abiotic Stress Assay Results

35S::G3516 (direct promoter fusion) lines were generally slightly smaller than control plants. In abiotic stress assays, five of ten lines were more tolerant to salt than controls in a germination assay. G3516 overexpression also increased tolerance to growth on nitrogen-limiting conditions. In the root growth assay under conditions of limiting nitrogen, 35S::G1792 lines were less stunted than controls. In the germination assay that monitors the effect of carbon on nitrogen signaling through anthocyanin production on media with high sucrose and with or without glutamine, the 35S::3516 lines made less anthocyanin on high sucrose with glutamine, indicating that this sequence is likely involved in monitoring carbon and nitrogen status in plants.

TABLE 31
35S::G3516 plate assay results

| | | | | | | | | | | Nitrogen utilization assays | | |
|---|---|---|---|---|---|---|---|---|---|---|---|---|
| | | | | | | Heat and cold assays | | | | Low | Low N | Low N |
| | Hyperosmotic stress assays | | | | | Heat | Cold | Heat | Cold | N | w/gln | root |
| Line | NaCl | Man | Suc | ABA | Dsc | germ | germ | growth | growth | germ | germ | growth |
| 301 | | | | | | | + | | | | + | |
| 302 | | | | | | | + | | | | + | |
| 303 | | | | | | | + | | | | + | |
| 304 | | | | | + | | + | | | + | + | |
| 305 | | | | | | | + | | | | + | + |
| 306 | | | | | | | + | | | | | |
| 307 | | | | | + | | + | | | | | |
| 308 | | | | | | | + | | | | | |
| 309 | | | | | | | + | | | + | + | |
| 310 | | | | + | | | + | | | + | + | + |

G3517 (Zea mays; SEQ ID NO: 19 and 20) Abiotic Stress Assay Results

At later stages of development 35S::G3517 lines were somewhat small in size with narrow leaves, but the plants are otherwise normal. Three out of ten lines 35S::G3517 direct promoter fusion lines performed better than wild-type seedlings in either a heat germination assay or under cold conditions in a growth assay.

TABLE 32
35S::G3517 plate assay results

| | | | | | | | | | | Nitrogen utilization assays | | |
|---|---|---|---|---|---|---|---|---|---|---|---|---|
| | | | | | | Heat and cold assays | | | | Low | Low N | Low N |
| | Hyperosmotic stress assays | | | | | Heat | Cold | Heat | Cold | N | w/gln | root |
| Line | NaCl | Man | Suc | ABA | Dsc | germ | germ | growth | growth | germ | germ | growth |
| 301 | | | | | | + | | | | | | |
| 302 | | | | | | | | | | | | |
| 305 | | | | | | + | | | + | | | |
| 308 | | | | | + | | | | | | | |
| 310 | | | | | + | | | | + | | | |
| 311 | | | | | | | | | + | | | |
| 312 | | | | | | + | | | | | | + |
| 318 | | | | | | | | | | | | |
| 319 | | | | | | | + | | | | | |
| 320 | | | | | | | + | | | | | |

G3518 (Glycine max; SEQ ID NO: 21 and 22) Abiotic Stress Assay Results

Several 35S::G3518 (direct promoter fusion) lines were small and dark green, but others showed no consistent differences relative to wild-type controls. A number of lines performed better than wild-type seedlings in germination assays in the presence of NaCl and cold. These same lines also did well in a growth assay under cold conditions, in low nitrogen conditions, and in a C/N sensing assay. Several lines performed poorly in a heat growth assay. Seedlings flowered earlier, suggesting they were stressed relative to wild-type and several had brown roots.

TABLE 33
35S::G3518 plate assay results

| | | | | | | | | | | Nitrogen utilization assays | | |
|---|---|---|---|---|---|---|---|---|---|---|---|---|
| | | | | | | Heat and cold assays | | | | Low | Low N | Low N |
| | Hyperosmotic stress assays | | | | | Heat | Cold | Heat | Cold | N | w/gln | root |
| Line | NaCl | Man | Suc | ABA | Dsc | germ | germ | growth | growth | germ | germ | growth |
| 301 | | | | | | | + | | | | | |
| 302 | + | | | | | | + | | + | + | + | |
| 303 | | | | | | | | | | | | |
| 305 | | | | | | | | | | | | |
| 307 | | | | | | | | | | | | |
| 321 | + | | | | + | | + | | | + | + | |
| 323 | + | | + | | | | + | − | + | + | + | |
| 325 | | | | | | | + | − | | | | |
| 326 | | | | | | | | − | + | | | |
| 327 | + | | | | | | | − | + | | | |
| 328 | + | | | | | | | − | + | + | + | |
| 330 | | | | | | | | − | + | | | |
| 331 | | | | | | | | − | + | | | |
| 332 | + | | | | | | + | | + | + | + | |
| 333 | + | | | | | | + | − | + | + | + | |

Three 35S::G3518 lines exhibited markedly enhanced drought tolerance compared to wild-type, both in terms of appearance at the end of the drought period, and in survivability following re-watering. Asterisks indicate statistically significant performance of experimental lines over controls (lines performed better than control; significant at P<0.11).

TABLE 34
Performance of 35S::G3518 (Glycine max) lines in soil-based drought assays

| | | | | | Evaluation after rewatering | | |
|---|---|---|---|---|---|---|---|
| | | Evaluation after drought treatment | | | Mean survival | Mean | |
| | | Mean score, | Mean | P value | for | survival | P value for |
| | Project | experimental | score, | score, | experimental | for | difference in |
| Line | Type | line | control | difference | line | control | survival |
| 323 | DPF | 2.0 | 1.4 | 0.053* | 0.37 | 0.33 | 0.45 |
| 323 | DPF | 1.3 | 0.50 | 0.0082* | 0.25 | 0.086 | 0.00042* |
| 326 | DPF | 1.7 | 1.6 | 0.53 | 0.34 | 0.34 | 0.90 |
| 326 | DPF | 0.70 | 0.50 | 0.40 | 0.11 | 0.050 | 0.082* |
| 333 | DPF | 2.1 | 2.1 | 0.87 | 0.39 | 0.42 | 0.63 |
| 333 | DPF | 1.3 | 0.60 | 0.043* | 0.23 | 0.12 | 0.020* |

G3520 (Glycine max; SEQ ID NO: 25 and 26) Abiotic Stress Assay Results

The majority of 35S::G3520 plants were small, late flowering, and had glossy, curled narrow leaves. Four our of seven 35S::G3520 direct promoter fusion lines performed better than wild-type control seedlings in a C/N sensing assay. A number of these lines also did well in a growth assay under low nitrogen and cold conditions.

TABLE 35
35S::G3520 plate assay results

| | | | | | | | | | | Nitrogen utilization assays | | |
|---|---|---|---|---|---|---|---|---|---|---|---|---|
| | | | | | | Heat and cold assays | | | | Low | Low N | Low N |
| | Hyperosmotic stress assays | | | | | Heat | Cold | Heat | Cold | N | w/gln | root |
| Line | NaCl | Man | Suc | ABA | Dsc | germ | germ | growth | growth | germ | germ | growth |
| 321 | | | | | | + | | | + | + | + | + |
| 325 | | | | | | | | | + | + | + | + |
| 345 | | | | + | | | | | | | | |
| 361 | | | | | | | | | | + | + | |
| 369 | | | | | | | | | | + | + | |
| 371 | | | | | | | | | | | | |
| 372 | | | | | | | | | | | | |

G3737 (Oryza saliva; SEQ ID NO: 31 and 32) Abiotic Stress Assay Results

A number of 35S::G3737 lines were small and late developing relative to controls, and at later stages of development some plants were late flowering and bushy with stems bent at nodes. A few lines were relatively normal in appearance and development. All 35S::G3737 direct promoter fusion lines tested germinated better than wild-type seedlings at 8° C. Five of these lines also germinated better than controls in high salt, five lines did better than controls in the sever desiccation assay, and three lines performed better than controls when grown at 8° C.

TABLE 36
35S::G3737 plate assay results

| | | | | | | | | | | Nitrogen utilization assays | | |
|---|---|---|---|---|---|---|---|---|---|---|---|---|
| | | | | | | Heat and cold assays | | | | Low | Low N | Low N |
| | Hyperosmotic stress assays | | | | | Heat | Cold | Heat | Cold | N | w/gln | root |
| Line | NaCl | Man | Suc | ABA | Dsc | germ | germ | growth | growth | germ | germ | growth |
| 301 | | | | + | | | + | | | | + | |
| 302 | | | | + | | | + | | | | | |
| 303 | + | | | + | | | + | | + | | | |
| 304 | + | | | + | + | | + | | | | | |
| 307 | | | | + | + | | + | | | | | |
| 308 | | | | + | + | | + | | | | | |
| 309 | + | | | + | + | | + | | | | | |
| 311 | + | | | + | | | + | | + | | | |
| 321 | | | | + | + | | + | | + | | + | |
| 323 | + | | | + | | | + | | | | | |

Three 35S::G3737 lines exhibited markedly enhanced drought tolerance compared to wild-type, both in terms of appearance at the end of the drought period, and in survivability following re-watering. Asterisks indicate statistically significant performance of experimental lines over controls (lines performed better than control; significant at P<0.11).

TABLE 37
Performance of 35S::G3737 (Oryza sativa) lines in soil-based drought assays

| | | | | | Evaluation after rewatering | | |
|---|---|---|---|---|---|---|---|
| | | Evaluation after drought treatment | | | Mean survival | Mean | |
| | | Mean score, | Mean | P value | for | survival | P value for |
| | Project | experimental | score, | score, | experimental | for | difference in |
| Line | Type | line | control | difference | line | control | survival |
| 304 | DPF | 2.5 | 1.7 | 0.011* | 0.52 | 0.36 | 0.0059* |
| 304 | DPF | 1.2 | 0.50 | 0.034* | 0.29 | 0.10 | 0.000097* |
| 308 | DPF | 2.8 | 1.6 | 0.00041* | 0.56 | 0.37 | 0.0020* |
| 308 | DPF | 1.7 | 0.90 | 0.041* | 0.31 | 0.16 | 0.0037* |
| 309 | DPF | 1.8 | 1.1 | 0.094* | 0.35 | 0.29 | 0.31 |
| 309 | DPF | 2.1 | 1.1 | 0.027* | 0.41 | 0.24 | 0.0016* |

G3739 (Zea maes; SEQ ID NO: 33 and 34) Abiotic Stress Assay Results

Some of the Arabidopsis lines overexpressing G3739 under the regulatory control of the 35S promoter (constitutive) were small and dark green. This promoter-gene combination conferred greater tolerance to mannitol, sucrose, ABA, desiccation, and germination in cold conditions than wild type. Overexpressors also performed better than controls in one nitrogen utilization assay although three lines appeared to be more sensitive than the controls to low nitrogen conditions in a root growth analysis.

TABLE 38
35S::G3739 plate assay results

| | | | | | | | | | | | Nitrogen utilization assays | | |
|---|---|---|---|---|---|---|---|---|---|---|---|---|---|
| | | | | | | | Heat and cold assays | | | | Low | Low N | Low N |
| Project | | Hyperosmotic stress assays | | | | | Heat | Cold | Heat | Cold | N | w/gln | root |
| Type | Line | NaCl | Man | Suc | ABA | Dsc | germ | germ | growth | growth | germ | germ | growth |
| DPF | 301 | | | + | + | | | | n/d | | | | |
| DPF | 302 | | + | | + | + | | + | n/d | | | + | |
| DPF | 303 | | + | | | + | | + | n/d | | | + | − |
| DPF | 304 | | + | | + | + | | + | n/d | | | + | − |
| DPF | 321 | | + | | | | | | n/d | | | | |
| DPF | 323 | | + | | | + | | + | n/d | | | + | |
| DPF | 324 | | | + | + | + | | | n/d | | | + | |
| DPF | 325 | | | | + | + | | + | n/d | | | + | |
| DPF | 330 | | + | | + | | | | n/d | | | | |
| DPF | 331 | | + | | | | | + | n/d | | | + | − |
| DPF | 335 | | + | | | + | | + | n/d | | | + | |
| DPF | 336 | | + | + | | + | | + | n/d | | | + | |

G3794 (Zea mays; SEQ ID NO: 35 and 36) Abiotic Stress Assay Results

A few of the Arabidopsis lines overexpressing G3794 under the regulatory control of the 35S promoter (constitutive) were spindly and small at various stages of development, and most of the plants were similar to wild type in morphology and slightly early developing as compared to wild-type controls. This promoter-gene combination conferred greater tolerance to desiccation and germination in cold conditions than wild type. One overexpressor line performed better than controls in a nitrogen utilization assay although three lines appeared to be more sensitive than the controls to low nitrogen conditions in a root growth analysis.

TABLE 39
35S::G3794 plate assay results

| | | | | | | | | | | | Nitrogen utilization assays | | |
|---|---|---|---|---|---|---|---|---|---|---|---|---|---|
| | | | | | | | Heat and cold assays | | | | Low | Low N | Low N |
| Project | | Hyperosmotic stress assays | | | | | Heat | Cold | Heat | Cold | N | w/gln | root |
| Type | Line | NaCl | Man | Suc | ABA | Dsc | germ | germ | growth | growth | germ | germ | growth |
| DPF | 302 | | | | | + | | | | | | | |
| DPF | 303 | | | | | | | + | | | | | − |
| DPF | 304 | | | + | | | | + | | | | | − |
| DPF | 305 | | | | | + | | + | | | | | |
| DPF | 306 | | | | | + | | + | | | | + | − |
| DPF | 307 | | | | | + | | + | | | | | |
| DPF | 308 | | | | | + | | + | | | | | |
| DPF | 309 | | | | | | | + | | | | | − |
| DPF | 310 | | | | | + | | | | | | | |
| DPF | 311 | | | | | | | + | | | | | − |

Dexamethasone-Inducible G1792 (Arabidopsis thaliana; SEQ ID NO: 1 and 2) Abiotic Stress Assay Results

Dexamethasone-inducible G1792 lines were similar to wild type in morphology and development to wild-type controls. This expression system conferred greater tolerance to desiccation than wild type. Four lines also performed better than controls in the root growth assay under low nitrogen conditions.

TABLE 40
Dexamethasone-inducible G1792 plate assay results

| | | | | | | | | | | | Nitrogen utilization assays | | |
|---|---|---|---|---|---|---|---|---|---|---|---|---|---|
| | | | | | | | Heat and cold assays | | | | Low | Low N | Low N |
| Project | | Hyperosmotic stress assays | | | | | Heat | Cold | Heat | Cold | N | w/gln | root |
| Type | Line | NaCl | Man | Suc | ABA | Dsc | germ | germ | growth | growth | germ | germ | growth |
| TCST | 323 | | | | | | | | | | | | |
| TCST | 334 | | | | | | | | | | | | |
| TCST | 1181 | | | | | | | | | | | | |
| TCST | 1182 | | | | | + | | | | | | | |
| TCST | 1183 | | | | | + | | | | | | | |
| TCST | 1184 | | | | | | | | | | | | + |
| TCST | 1185 | | | | | + | | | | | | | + |
| TCST | 1186 | | + | | | | | | | | | | |
| TCST | 1187 | | | | | | | | | | | | + |
| TCST | 1188 | | | | | + | | | | | | | |
| TCST | 1189 | | | | | + | | | | | | | |
| TCST | 1190 | | + | | | | | | | | | | + |

Utilities for G1792 clade members under constitutive and non-constitutive regulatory control. The results of these studies with the constitutive and non-constitutive regulatory control of many G1792 clade members indicate that the polynucleotide and polypeptide sequences can be used to improve abiotic stress tolerance, and in a number of cases can do so without conferring severe adverse morphological or developmental defects to the plants. These data confirm our conclusions that G1792 and other G1792 clade members may be valuable tools for the purpose of increasing yield and quality of plant products.

### Example IX

### Results Identifying Genes that Confer Significant Disease Tolerance

This example provides experimental evidence for increased disease tolerance controlled by the transcription factor polypeptides and polypeptides of the invention. The transcription factor sequences of the Sequence Listing can be used to prepare transgenic plants with altered traits. From the experimental results of the plate-based and growth assays-presented in the tables of this Example, it may be inferred that a representative number of sequences from diverse plant species imparted increased disease tolerance to a number of pathogens. These comparable effects indicate that sequences found within the G1792 clade of transcription factor polypeptides are functionally related and can be used to confer various types of disease stress tolerance in plants. A number of these genes conferred increased tolerance to multiple pathogens.

As determined from experimental assays, a number of members of the G1792 clade of transcription factor polypeptides from diverse plant species, including G1792 (SEQ ID NO: 2), G1791 (SEQ ID NO: 4), G1795 (SEQ ID NO: 6), G30 (SEQ ID NO: 8), G3381 (SEQ ID NO: 12), G3517 (SEQ ID NO: 20) and G3520 (SEQ ID NO: 26), increase disease tolerance when these sequences are overexpressed.

In initial studies, 35S::G1792 plants were found to be more tolerant to the fungal pathogens Fusarium oxysporum and Botrytis cinerea and showed fewer symptoms after inoculation with a low dose of each pathogen. This result was confirmed using individual T2 lines. The effect of G1792 overexpression in increasing tolerance to pathogens received further, incidental confirmation. T2 plants of two 35S::G1792 lines had been growing in a room that suffered a serious powdery mildew infection. For each line, a pot of six plants was present in a flat containing nine other pots of lines from unrelated genes. In either of the two different flats, the only plants that were free from infection (that is, showing a 100% reduction in symptoms) were those from the 35S::G1792 line. This observation suggested that G1792 overexpression may be used to increase resistance to powdery mildew. Additional experiments confirmed that 35S::G1792 plants showed significantly increased tolerance to Erysiphe; a significant number of these plants had exhibited a 100% reduction in disease symptoms, and appeared to be disease-free. G1792 was ubiquitously expressed, but appeared to be induced by salicylic acid.

We then predicted that other sequences within the G1792 clade may also confer similar functions, including disease tolerance, based on the phylogenetic relatedness and structural similarities of these sequences. A summary of the disease assay results for four Arabidopsis sequences and two non-Arabidopsis sequences in this clade is presented in Table 41. At least seven sequences in the clade derived from diverse species, including three non-Arabidopsis orthologs, G3520 (soybean), G3517 (maize) and G3381 (rice), provided significantly enhanced tolerance to Sclerotinia and/or powdery mildew when overexpressed in Arabidopsis using various regulatory controls. Many of the plants overexpressing G1792 paralogs showed a considerable reduction in disease symptoms, and a number appeared to be 100% free.

TABLE 41
Disease screening of various G1792 paralogs and orthologs (GID; polynucleotide
SEQ ID NO, polypeptide SEQ ID NO) under different expression systems

| | G1792; 1, 2 | | | | G1791; 3, 4 | | | | G1795; 5, 6 | | | | G30; 7, 8 | | | | G3381; 11, 12 | | | | G3520; 25, 26 | | | | G3517; 19, 20 | | | |
|---|---|---|---|---|---|---|---|---|---|---|---|---|---|---|---|---|---|---|---|---|---|---|---|---|---|---|---|---|
| | B | S | F | P | B | S | F | P | B | S | F | P | B | S | F | P | B | S | F | P | B | S | F | P | B | S | F | P |
| 35S | ++ | wt | + | + | | | | | | | | | | | | | | + | | + | | + | | | | + | | + |
| RBCS3 | + | wt | + | | wt | wt | wt | | ++ | ++ | wt | | + | + | wt | | | | | | | | | | | | | |
| LTP1 | wt | wt | | | + | wt | wt | | ++ | + | wt | | wt | wt | wt | | | | | | | | | | | | | |
| CUT1 | | | | | + | + | | | | + | | + | | | | | | | | | | | | | | | | |
| SUC2 | | | | | | | | | | + | | | | | | | | | | | | | | | | | | |
| Dex-ind. | ++ | wt | + | | ++ | ++ | wt | | ++ | ++ | wt | | ++ | ++ | wt | | | | | | | | | | | | | |

Abbreviations:
B Botrytis cinerea
S Sclerotinia sclerotiorum
F Fusarium oxysporum
P Powdery mildew
Scoring:
++ significant improvement in tolerance
+ mild to moderate improvement in tolerance
wt no difference in tolerance from wild-type controls (susceptible)
empty cell: not done

The results of these studies and those of the previous example indicate that constitutive and non-constitutive constitutive regulatory control of a significant number of G1792 clade member polynucleotides can be used to improve disease resistance.

### Example X

### Disease Resistance and Abiotic Stress Tolerance without Severe Developmental or Morphological Defects

As noted below, overexpression of G1792 and its closely-related homologs using non-constitutive regulatory schemes produced plants that were similar in their development and morphology to wild type, but which retained disease resistance and abiotic stress tolerant phenotypes.

SUC2::G1792 lines, including many lines that were positive in abiotic stress assays, were generally very similar in their development and morphology to wild-type controls.

Some STM::G1792 lines were smaller than controls. At least one STM::G1792 overexpressor that was positive in both mannitol and cold germination assays was similar to wild-type controls in its development and morphology. One other line that was positive in abiotic stress assays may have been somewhat delayed in development at a late stage.

A number of RBCS3:G1792 lines were late flowering, slightly small in size and slightly dark in coloration. All other lines were equivalent in morphology to control lines, including lines that were more tolerant to salt or more resistant to disease than wild-type controls.

Overall, LTP1::G1792 lines were not consistently different from control plants in their development and morphology.

At early stages of growth, some of the RSI1::G1792 two-component lines were small in size and/or early developing relative to wild-type controls. At later stages, almost all of the lines were similar in morphology to the control plants. Some lines, including some of those positive in the C/N sensing assay, showed no consistent differences relative to controls at any stage.

RD29A::G1792 lines were generally small through the rosette stage of development but were later similar to controls in their morphology and development.

Dexamethasone-inducible G1792 lines tested in disease assays were generally morphologically and developmentally similar to wild-type control plants.

RBCS3::G1791 and LTP1::G1791 lines were generally similar to control lines in their development and morphology (a few RBCS3::G1791 may have been slightly late in their development).

Dexamethasone-inducible G1791 lines tested in disease assays were generally morphologically and developmentally similar to wild-type control plants.

At early and later stages of growth, both LTP1::G1795 and RBCS3::G1795 overexpressors were similar in morphology to controls, including lines resistant to pathogens. These lines were slightly small relative to controls at the rosette stage of development, had dark green leaves, and all lines flowered late. LTP1::G 1795 lines also tended to be darker than control plants at the rosette stage.

SUC2::G1795 lines were generally smaller than wild-type controls, although at least one line was wild-type in its development and morphology.

Dexamethasone-inducible G1795 lines were generally smaller and dark green than wild-type controls, but the differences from the controls were much less severe than the effects seen in 35S:G1795 plants.

SUC2::G30 lines were generally dark, had shiny, curly leaves, and were small, relative to controls.

LTP1::G30 lines were slightly small and marginally darker green relative to control plants. At the flowering and later stages of growth, the plants were generally similar to wild-type.

Most of the RBCS3::G30 lines were marginally small and somewhat late in their development. All of these lines were at least marginally late flowering, and had dark green/slightly wrinkled leaves. At late stages of development almost all plants showed no consistent differences relative to wild-type controls. LTP1::G30 plants were similar in their development; all were dark in color, late developing and slightly small in size at early stages, slightly smaller than wild type at the rosette stage, and very similar to controls at late stages of development.

A number of RSI1::G30 lines were small, dark green and shiny with upright leaves. However, other lines, including some that were positive in cold tolerance germination assays showed no consistent differences relative to control plants.

RD29A::G30 lines, including lines that were positive in abiotic stress assays, ranged from small to wild-type in their morphology and development.

Dexamethasone-inducible G30 lines were generally smaller than wild-type control plants, but the differences from the controls were much less severe than the effects seen in 35S:G30 plants.

### Example XI

### Identification of Homologous Sequences by Computer Homology Search

This example describes identification of genes that are orthologous to Arabidopsis thaliana G1792 clade member transcription factors from a computer homology search.

Homologous sequences, including those of paralogs and orthologs from Arabidopsis and other plant species, were identified using database sequence search tools, such as the Basic Local Alignment Search Tool (BLAST) (Altschul et al. (1990) supra; and Altschul et al. (1997) Nucleic Acid Res. 25: 3389-3402). The tblastx sequence analysis programs were employed using the BLOSUM-62 scoring matrix (Henikoff and Henikoff (1992) Proc. Natl. Acad. Sci. USA 89: 10915-10919). The entire NCBI GenBank database was filtered for sequences from all plants except Arabidopsis thaliana by selecting all entries in the NCBI GenBank database associated with NCBI taxonomic ID 33090 (Viridiplantae; all plants) and excluding entries associated with taxonomic ID 3701 (Arabidopsis thaliana).

These sequences are compared to sequences representing transcription factor genes presented in the Sequence Listing, using the Washington University TBLASTX algorithm (version 2.0a19MP) at the default settings using gapped alignments with the filter “off”. For each transcription factor gene in the Sequence Listing, individual comparisons were ordered by probability score (P-value), where the score reflects the probability that a particular alignment occurred by chance. For example, a score of 3.6e-59 is 3.6×10-59. In addition to P-values, comparisons were also scored by percentage identity. Percentage identity reflects the degree to which two segments of DNA or protein are identical over a particular length. Examples of sequences so identified are presented in, for example, the Sequence Listing and Table 1. Paralogous or orthologous sequences may be readily identified and available in GenBank by Accession number (Sequence Identifier or Accession Number). The percent sequence identity among these sequences can be as low as 49%, or even lower sequence identity.

Candidate paralogous sequences were identified among Arabidopsis transcription factors through alignment, identity, and phylogenic relationships. G1791, G1795 and G30 (SEQ ID NO: 4, 6, and 8, respectively), paralogs of G1792, may be found in the Sequence Listing.

Candidate orthologous sequences were identified from proprietary unigene sets of plant gene sequences in Zea mays, Glycine max and Oryza sativa based on significant homology to Arabidopsis transcription factors. These candidates were reciprocally compared to the set of Arabidopsis transcription factors. If the candidate showed maximal similarity in the protein domain to the eliciting transcription factor or to a paralog of the eliciting transcription factor, then it was considered to be an ortholog. Identified non-Arabidopsis sequences that were shown in this manner to be orthologous to the Arabidopsis sequences are provided in, for example, Table 1.

### Example XII

### Transformation of Dicots

Crop species overexpressing members of the G1792 clade of transcription factor polypeptides have been shown experimentally to produce plants with increased tolerance to low nitrogen and abiotic stress (including hyperosmotic stress and/or heat and/or cold). This observation indicates that these genes, when overexpressed, will result in larger yields of various plant species, particularly during conditions of abiotic stress or low nitrogen.

Thus, transcription factor sequences listed in the Sequence Listing recombined into pMEN20 or pMEN65 expression vectors may be transformed into a plant for the purpose of modifying plant traits. The cloning vector may be introduced into a variety of cereal plants by means well known in the art such as, for example, direct DNA transfer or Agrobacterium tumefaciens-mediated transformation. It is now routine to produce transgenic plants using most dicot plants (see Weissbach and Weissbach, (1989) supra; Gelvin et al. (1990) supra; Herrera-Estrella et al. (1983) supra; Bevan (1984) supra; and Klee (1985) supra). Methods for analysis of traits are routine in the art and examples are disclosed above.

Methods for transforming cotton may be found in U.S. Pat. Nos. 5,004,863, 5,159,135 and 5,518,908; for transforming brassica species may be found in U.S. Pat. No. 5,463,174; for transforming peanut plants may be found in Cheng et al. (1996) Plant Cell Rep. 15: 653-657, and McKently et al. (1995) Plant Cell Rep. 14: 699-703; and for transforming pea may be found in Grant et al. (1995) Plant Cell Rep. 15: 254-258.

Numerous protocols for the transformation of tomato and soy plants have been previously described, and are well known in the art. Gruber et al. ((1993) in Methods in Plant Molecular Biology and Biotechnology, p. 89-119, Glick and Thompson, eds., CRC Press, Inc., Boca Raton) describe several expression vectors and culture methods that may be used for cell or tissue transformation and subsequent regeneration. For soybean transformation, methods are described by Miki et al. (1993) in Methods in Plant Molecular Biology and Biotechnology, p. 67-88, Glick and Thompson, eds., CRC Press, Inc., Boca Raton; and U.S. Pat. No. 5,563,055, (Townsend and Thomas), issued Oct. 8, 1996.

There are a substantial number of alternatives to Agrobacterium-mediated transformation protocols, other methods for the purpose of transferring exogenous genes into soybeans or tomatoes. One such method is microprojectile-mediated transformation, in which DNA on the surface of microprojectile particles is driven into plant tissues with a biolistic device (see, for example, Sanford et al., (1987) Part. Sci. Technol. 5:27-37; Christou et al. (1992) Plant. J. 2: 275-281; Sanford (1993) Methods Enzymol. 217: 483-509; Klein et al. (1987) Nature 327: 70-73; U.S. Pat. No. 5,015,580 (Christou et al), issued May 14, 1991; and U.S. Pat. No. 5,322,783 (Tomes et al.), issued Jun. 21, 1994.

Alternatively, sonication methods (see, for example, Zhang et al. (1991) Bio/Technology 9: 996-997); direct uptake of DNA into protoplasts using CaCl₂ precipitation, polyvinyl alcohol or poly-L-ornithine (see, for example, Hain et al. (1985) Mol. Gen. Genet. 199: 161-168; Draper et al., Plant Cell Physiol. 23: 451-458 (1982)); liposome or spheroplast fusion (see, for example, Deshayes et al. (1985) EMBO J., 4: 2731-2737; Christou et al. (1987) Proc. Natl. Acad. Sci. USA 84: 3962-3966); and electroporation of protoplasts and whole cells and tissues (see, for example, Donn et al. (1990) in Abstracts of VIIth International Congress on Plant Cell and Tissue Culture IAPTC, A2-38: 53; D'Halluin et al. (1992) Plant Cell 4: 1495-1505; and Spencer et al. (1994) Plant Mol. Biol. 24: 51-61) have been used to introduce foreign DNA and expression vectors into plants.

After a plant or plant cell is transformed (and the latter regenerated into a plant), the transformed plant may be crossed with itself or a plant from the same line, a non-transformed or wild-type plant, or another transformed plant from a different transgenic line of plants. Crossing provides the advantages of producing new and often stable transgenic varieties. Genes and the traits they confer that have been introduced into a tomato or soybean line may be moved into distinct line of plants using traditional backcrossing techniques well known in the art. Transformation of tomato plants may be conducted using the protocols of Koornneef et al (1986) In Tomato Biotechnology: Alan R. Liss, Inc., 169-178, and in U.S. Pat. No. 6,613,962, the latter method described in brief here. Eight day old cotyledon explants are precultured for 24 hours in Petri dishes containing a feeder layer of Petunia hybrida suspension cells plated on MS medium with 2% (w/v) sucrose and 0.8% agar supplemented with 10 μM α-naphthalene acetic acid and 4.4 μM 6-benzylaminopurine. The explants are then infected with a diluted overnight culture of Agrobacterium tumefaciens containing an expression vector comprising a polynucleotide of the invention for 5-10 minutes, blotted dry on sterile filter paper and cocultured for 48 hours on the original feeder layer plates. Culture conditions are as described above. Overnight cultures of Agrobacterium tumefaciens are diluted in liquid MS medium with 2% (wavy) sucrose, pH 5.7) to an OD₆₀₀ of 0.8.

Following cocultivation, the cotyledon explants are transferred to Petri dishes with selective medium comprising MS medium with 4.56 μM zeatin, 67.3 μM vancomycin, 418.9 μM cefotaxime and 171.6 μM kanamycin sulfate, and cultured under the culture conditions described above. The explants are subcultured every three weeks onto fresh medium. Emerging shoots are dissected from the underlying callus and transferred to glass jars with selective medium without zeatin to form roots. The formation of roots in a kanamycin sulfate-containing medium is a positive indication of a successful transformation.

Transformation of soybean plants may be conducted using the methods found in, for example, U.S. Pat. No. 5,563,055. In this method, soybean seed is surface sterilized by exposure to chlorine gas evolved in a glass bell jar. Seeds are germinated by plating on 1/10 strength agar solidified medium without plant growth regulators and culturing at 28° C. with a 16 hour day length. After three or four days, seed may be prepared for cocultivation. The seedcoat is removed and the elongating radicle removed 3-4 mm below the cotyledons.

Overnight cultures of Agrobacterium tumefaciens harboring the expression vector comprising a polynucleotide of the invention are grown to log phase, pooled, and concentrated by centrifugation. Inoculations are conducted in batches such that each plate of seed was treated with a newly resuspended pellet of Agrobacterium. The pellets are resuspended in 20 ml inoculation medium. The inoculum is poured into a Petri dish containing prepared seed and the cotyledonary nodes are macerated with a surgical blade. After 30 minutes the explants are transferred to plates of the same medium that has been solidified. Explants are embedded with the adaxial side up and level with the surface of the medium and cultured at 22° C. for three days under white fluorescent light. These plants may then be regenerated according to methods well established in the art, such as by moving the explants after three days to a liquid counter-selection medium (see U.S. Pat. No. 5,563,055).

The explants may then be picked, embedded and cultured in solidified selection medium. After one month on selective media transformed tissue becomes visible as green sectors of regenerating tissue against a background of bleached, less healthy tissue. Explants with green sectors are transferred to an elongation medium. Culture is continued on this medium with transfers to fresh plates every two weeks. When shoots are 0.5 cm in length they may be excised at the base and placed in a rooting medium.

### Example XIII

### Increased Biotic and Abiotic Stress Tolerance in Monocots

Cereal plants such as, but not limited to, corn, wheat, rice, sorghum, or barley, may be transformed with the present polynucleotide sequences, including monocot or dicot-derived sequences such as those presented in Tables 1 and 5-40, and other clade members that are not listed in the Sequence Listing but which may be identified as such using the methods disclosed herein, cloned into a vector such as pGA643 and containing a kanamycin-resistance marker, and expressed constitutively under, for example, the CaMV 35S or COR15 promoters. pMEN20 or pMEN65 and other expression vectors may also be used for the purpose of modifying plant traits. For example, pMEN020 may be modified to replace the NptII coding region with the BAR gene of Streptomyces hygroscopicus that confers resistance to phosphinothricin. The KpnI and BglII sites of the Bar gene are removed by site-directed mutagenesis with silent codon changes.

The cloning vector may be introduced into a variety of cereal plants by means well known in the art including direct DNA transfer or Agrobacterium tumefaciens-mediated transformation. The latter approach may be accomplished by a variety of means, including, for example, that of U.S. Pat. No. 5,591,616, in which monocotyledon callus is transformed by contacting dedifferentiating tissue with the Agrobacterium containing the cloning vector. The sample tissues are immersed in a suspension of 3×10⁻⁹ cells of Agrobacterium containing the cloning vector for 3-10 minutes. The callus material is cultured on solid medium at 25° C. in the dark for several days. The calli grown on this medium are transferred to Regeneration medium. Transfers are continued every 2-3 weeks (2 or 3 times) until shoots develop. Shoots are then transferred to Shoot-Elongation medium every 2-3 weeks. Healthy looking shoots are transferred to rooting medium and after roots have developed, the plants are placed into moist potting soil.

The transformed plants are then analyzed for the presence of the NPTII gene/kanamycin resistance by ELISA, using the ELISA NPTII kit from 5Prime-3Prime Inc. (Boulder, Colo.).

It is also routine to use other methods to produce transgenic plants of most cereal crops (Vasil (1994) Plant Mol. Biol. 25: 925-937) such as corn, wheat, rice, sorghum (Cassas et al. (1993) Proc. Natl. Acad. Sci. USA 90: 11212-11216, and barley (Wan and Lemeaux (1994) Plant Physiol. 104:37-48). DNA transfer methods such as the microprojectile method can be used for corn (Fromm et al. (1990) Bio/Technol. 8: 833-839); Gordon-Kamm et al. (1990) Plant Cell 2: 603-618; Ishida (1990) Nature Biotechnol. 14:745-750), wheat (Vasil et al. (1992) Bio/Technol. 10:667-674; Vasil et al. (1993) Bio/Technol. 1: 1553-1558; Weeks et al. (1993) Plant Physiol. 102:1077-1084), and rice (Christou (1991) Bio/Technol. 9:957-962; Hiei et al. (1994) Plant J. 6:271-282; Aldemita and Hodges (1996) Planta 199:612-617; and Hiei et al. (1997) Plant Mol. Biol. 35:205-218). For most cereal plants, embryogenic cells derived from immature scutellum tissues are the preferred cellular targets for transformation (Hiei et al. (1997) Plant Mol. Biol. 35:205-218; Vasil (1994) Plant Mol. Biol. 25: 925-937). For transforming corn embryogenic cells derived from immature scutellar tissue using microprojectile bombardment, the A188XB73 genotype is the preferred genotype (Fromm et al. (1990) Bio/Technol. 8: 833-839; Gordon-Kamm et al. (1990) Plant Cell 2: 603-618). After microprojectile bombardment the tissues are selected on phosphinothricin to identify the transgenic embryogenic cells (Gordon-Kamm et al. (1990) Plant Cell 2: 603-618). Transgenic plants are regenerated by standard corn regeneration techniques (Fromm et al. (1990) Bio/Technol. 8: 833-839; Gordon-Kamm et al. (1990) Plant Cell 2: 603-618).

Northern blot analysis, RT-PCR or microarray analysis of the regenerated, transformed plants may be used to show expression of G1792 and related genes that are capable of conferring tolerance to biotic or abiotic stress.

To verify the ability to confer abiotic stress tolerance, mature plants overexpressing a G1792 clade member, or alternatively, seedling progeny of these plants, may be challenged by low nitrogen conditions or another abiotic stress such as heat, cold, or the hyperosmotic stresses of drought, high salt or freezing. Alternatively, these plants may be challenged in an osmotic stress condition that may also measure altered sugar sensing, such as a high sugar condition. In another alternative series of assays, these plants may be challenged with various pathogens and selected for disease resistance. By comparing wild type and transgenic plants similarly treated, the transgenic plants may be shown to have greater tolerance to biotic and or abiotic stress.

By comparing wild type and transgenic plants similarly treated, the transgenic plants may be shown to have greater disease resistance or tolerance to low nitrogen conditions and/or abiotic stress, or also fewer adverse effects from low nitrogen conditions and/or abiotic stresses including hyperosmotic, heat, and cold stresses.

The transgenic plants may also have greater yield relative to a control plant when both are faced with the same low nitrogen or abiotic stress. Since plants overexpressing members of the G1792 clade may be tolerant to one or more abiotic stresses, plants overexpressing a member of the G1792 clade may incur a smaller yield loss and better quality than control plants when the overexpressors and control plants are faced with similar abiotic stress challenges. Better yield or quality may be obtained by, for example, reducing distortions, lesion size or number, defoliation, stunting, necrosis or pathogen susceptibility (e.g., pathogen growth or sporulation) by at least about 5%, or at least 10%, or at least 20% or more, up to 100%, relative to a control plant exposed to the same abiotic stress, or increasing chlorophyll content or photosynthesis by at least about 5%, or at least 10%, or at least 20% or more relative to a control plant subjected to the same abiotic stress. As indicated in Example VIII, a number of plants overexpressing members of the G1792 clade showed significantly better turgor and greater mass (up to and including 100%) and significantly fewer or reduced stress-related symptoms compared to control plants.

After a monocot plant or plant cell is transformed (and the latter regenerated into a plant) and shown to have greater disease resistance or tolerance to low nitrogen and/or abiotic stress, or produce greater yield relative to a control plant under the stress conditions, the transformed monocot plant may be crossed with itself or a plant from the same line, a non-transformed or wild-type monocot plant, or another transformed monocot plant from a different transgenic line of plants.

### Example XIV

### Sequences that Confer Significant Improvements to Non-Arabidopsis Species

The function of specific orthologs of G1792 has been analyzed and may be further characterized by incorporation into crop plants. The ectopic overexpression of these orthologs may be regulated using constitutive, inducible, or tissue specific regulatory elements, as disclosed above. Genes that have been examined and have been shown to modify plant traits (including increasing resistance to various diverse diseases, or tolerance to one or more abiotic stressed or multiple abiotic stresses) encode members of the G1792 clade of transcription factor polypeptides, such as those found in Arabidopsis thaliana (SEQ ID NO: 2, 4, 6 and 8), Glycine max (22, 24, and 26), Medicago truncatula (28), Oryza saliva (SEQ ID NO: 10, 12, 14, 16, and 32), Triticum aestivum (30), and Zea mays (SEQ ID NO: 18, 20, 34 and 36). In addition to these sequences, it is expected that related polynucleotide sequences encoding polypeptides found in the Sequence Listing can also induce increased tolerance to abiotic stresses, when transformed into a considerable variety of plants of different species, and including higher plants. The polynucleotide and polypeptide sequences in the sequence listing may be used to transform any higher plant. For example, sequences derived from monocots (e.g., the rice or corn sequences) may be used to transform both monocot and dicot plants, and those derived from dicots (e.g., the Arabidopsis and soy genes) may be used to transform either group, although it is expected that some of these sequences will function best if the gene is transformed into a plant from the same group as that from which the sequence is derived.

In addition to the constitutive 35S promoter, G1792 clade members may be overexpressed under the regulatory control of inducible or tissue-specific promoters. For example, ARSK1 and RSI1 (root-specific), RBCS3 (photosynthetic tissue-specific), CUT1 and LTP1 (epidermal-specific), SUC2 (vascular-specific) STM (shoot apical meristem-specific), AP1 (floral meristem-specific), AS1 (emergent leaf primordia-specific) and RD29A (stress-inducible) promoters may be used to confer abiotic stress tolerance in plants. Typically, these promoter-gene combinations may be readily achieved via the two-component system, although direct promoter fusions may also be considered. To date, we have found the use of alternative tissue-specific promoters to be a particular valuable approach in dissecting and optimizing gene function. In a number of cases, we have found that a stress-tolerance phenotype could be achieved without undesirable morphological changes (e.g., stunting, low fertility) that may be conferred when using a constitutive promoter.

These experiments demonstrate that a number of G1792 clade members, including G30, G1791, and G1792, soybean G3518 and G3520, rice G3380, G3381, G3383, G3515, and G3737, and corn G3516 and G3517 (SEQ ID NO: 8, 4, 2, 22, 26, 10, 12, 14, 16, 32, 18, and 20, respectively) can be identified and shown to confer increased disease resistance and abiotic stress tolerance in a plant relative to a control plant. It is expected that the same methods may be applied to identify and eventually make use of other members of the clade from a diverse range of species.

All publications and patent applications mentioned in this specification are herein incorporated by reference to the same extent as if each individual publication or patent application was specifically and individually indicated to be incorporated by reference.

The present invention is not limited by the specific embodiments described herein. The invention now being fully described, it will be apparent to one of ordinary skill in the art that many changes and modifications can be made thereto without departing from the spirit or scope of the appended claims. Modifications that become apparent from the foregoing description and accompanying figures fall within the scope of the claims.

What is claimed is:

1. A transgenic plant transformed with an expression vector comprising a polynucleotide encoding a member of the G1792 clade of transcription factor polypeptides comprising:
an AP2 domain, and
an EDLL domain of SEQ ID NO: 63; wherein
the expression vector further comprises a regulatory element operably linked to the polynucleotide;
the transgenic plant is more tolerant to drought, salt, cold, heat, or low nitrogen conditions than a wild type control plant and/or more resistant to a disease pathogen than the wild type control plant; and
the transgenic plant is morphologically and/or developmentally similar to the wild type control plant.

2. The transgenic plant of claim 1, wherein the regulatory element is selected from the group consisting of a vascular tissue-specific promoter, a root tissue-specific promoter, a photosynthetic tissue-specific promoter, an epidermal-tissue specific promoter, a shoot apical meristem-specific promoter, and a stress-inducible promoter.

3. The transgenic plant of claim 1, wherein a GAL4 activation domain is fused to the member of the G1792 clade of transcription factor polypeptides to create a terminal GAL4 activation domain protein fusion.

4. The transgenic plant of claim 1, wherein the transgenic plant is resistant to at least one fungal disease.

5. The transgenic plant of claim 4, wherein the transgenic plant is resistant to Sclerotinia, Fusarium, Botrytis or powdery mildew.

6. The transgenic plant of claim 1, wherein the transgenic plant is resistant to more than one pathogen or disease.

7. The transgenic plant of claim 1, wherein the transgenic plant is more tolerant to more than one abiotic stress.

8. The transgenic plant of claim 1, wherein the AP2 domain and the EDLL domain are at least 70% and 62% identical to the AP2 domain and the EDLL domain of SEQ ID NO: 2, respectively

9. A method for producing an abiotic stress tolerant or disease resistant plant that is morphologically and/or developmentally similar to a wild-type control plant; wherein
the abiotic stress is selected from the group consisting of desiccation, drought, cold, heat, and low nitrogen conditions;
the method steps comprising:
(a) transforming a target plant with an expression vector comprising a polynucleotide encoding a member of the G1792 clade of transcription factor polypeptides comprising an AP2 domain and an EDLL domain of SEQ ID NO: 63; wherein
the expression vector further comprises a regulatory element operably linked to the polynucleotide; and
(b) selecting a transgenic plant that is more tolerant to the abiotic stress or disease than a wild type control plant, and is also morphologically and/or developmentally similar to the wild type control plant.

10. The method of claim 9, wherein the regulatory element is selected from the group consisting of a vascular tissue-specific promoter, a root tissue-specific promoter, a photosynthetic tissue-specific promoter, an epidermal-tissue specific promoter, a shoot apical meristem-specific promoter, and a stress-inducible promoter.

11. The method of claim 9, wherein the transgenic plant is tolerant to at least one abiotic stress and resistant to at least one disease pathogen.

12. A method for increasing the disease resistance of a plant of wild-type morphology and/or development, the method steps comprising:
(a) transforming a target plant with an expression vector comprising a polynucleotide encoding a member of the G1792 clade of transcription factor polypeptides comprising an AP2 domain and an EDLL domain of SEQ ID NO: 63; wherein
the expression vector further comprises a regulatory element operably linked to the polynucleotide; and
(b) selecting a transgenic plant that is more tolerant to the disease than a wild type control plant and is morphologically and/or developmentally similar to a wild type control plant.

13. The method of claim 12, wherein the regulatory element is selected from the group consisting of a vascular tissue-specific promoter, a root tissue-specific promoter, a photosynthetic tissue-specific promoter, an epidermal-tissue specific promoter, a shoot apical meristem-specific promoter, and a stress-inducible promoter.

14. The method of claim 12, wherein a GAL4 activation domain is fused to the member of the G1792 clade of transcription factor polypeptides to create a terminal GAL4 activation domain protein fusion.

15. The method of claim 12, wherein the AP2 domain and the EDLL domain are at least 70% and 62% identical to the AP2 domain and the EDLL domain of SEQ ID NO: 2, respectively

16. A method for increasing the abiotic stress tolerance of a plant of wild-type morphology and/or development, wherein the abiotic stress is selected from the group consisting of drought, salt, cold, heat, and low nitrogen conditions, the method steps comprising:
(a) transforming a target plant with an expression vector comprising a polynucleotide encoding a member of the G1792 clade of transcription factor polypeptides comprising an AP2 domain and an EDLL domain of SEQ ID NO: 63; wherein
the expression vector further comprises a regulatory element operably linked to the polynucleotide; and
(b) selecting a transgenic plant that is more tolerant to the abiotic stress than a wild type control plant and is morphologically and/or developmentally similar to a wild type control plant.

17. The method of claim 16, wherein the regulatory element is selected from the group consisting of a vascular tissue-specific promoter, a root tissue-specific promoter, a photosynthetic tissue-specific promoter, an epidermal-tissue specific promoter, a shoot apical meristem-specific promoter, and a stress-inducible promoter.

18. The method of claim 16, wherein a GAL4 activation domain is fused to the member of the G1792 clade of transcription factor polypeptides to create a terminal GAL4 activation domain protein fusion.

19. The method of claim 16, wherein the AP2 domain and the EDLL domain are at least 70% and 62% identical to the AP2 domain and the EDLL domain of SEQ ID NO: 2, respectively.

20. A seed produced by a transgenic plant made by the method of claim 9, wherein the seed comprises a polynucleotide encoding a member of the G1792 clade of transcription factor polypeptides comprising an AP2 domain and an EDLL domain of SEQ ID NO: 63.

21. A transgenic plant that is morphologically and/or developmentally similar to a wild-type plant of the same species, where the transgenic plant has been transformed with an expression vector comprising a polynucleotide encoding an AP2 domain that is at least 70% identical to the AP2 domain of SEQ ID NO: 2 and an EDLL domain that is 62% identical to the EDLL domain of SEQ ID NO: 2.


SEQUENCE LISTING (79 sequences)

SEQ ID NO: 1 - DNA, 696 bp, Arabidopsis thaliana
  G1792
aatccatagatctcttattaaataacagtgctgaccaagctcttacaaagcaaaccaatctagaacaccaaagttaatggagagctcaaacaggagcagcaacaaccaatcacaagatgacaagcaagctcgtttccggggagttcgaagaaggccttggggaaagtttgcagcagagattcgagacccgtcgagaaacggtgcccgtctttggctcgggacatttgagaccgctgaggaggcagcaagggcttatgaccgagcagcctttaaccttaggggtcatctcgctatactcaacttccctaatgagtattatccacgtatggacgactactcgcttcgccctccttatgcttcttcttcttcgtcgtcgtcatcgggttcaacttctactaatgtgagtcgacaaaaccaaagagaagttttcgagtttgagtatttggacgataaggttcttgaagaacttcttgattcagaagaaaggaagagataatcacgattagttttgttttgatattttatgtggcactgttgtggctacctacgtgcattatgtgcatgtataggtcgcttgattagtactttataacatgcatgccacgaccataaattgtaagagaagacgtactttgcgttttcatgaaatatgaatgttagatggtttgagtacaaaaaaaaaaaaaaaaaaaaaaa

SEQ ID NO: 2 - protein, 139 aa, Arabidopsis thaliana
  G1792 polypeptide
MESSNRSSNNQSQDDKQARFRGVRRRPWGKFAAEIRDPSRNGARLWLGTFETAEEAARAYDRAAFNLRGHLAILNFPNEYYPRMDDYSLRPPYASSSSSSSSGSTSTNVSRQNQREVFEFEYLDDKVLEELLDSEERKR

SEQ ID NO: 3 - DNA, 549 bp, Arabidopsis thaliana
  G1791
atgtacatgcaaaaacaaaaaccttaaaagctttcatggaacgtatagagtcttataacacgaatgagatgaaatacagaggcgtacgaaagcgtccatggggaaaatatgcggcggagattcgcgactcagctagacacggtgctcgtgtttggcttgggacgtttaacacagcggaagacgcggctcgggcttatgatagagcagctttcggcatgagaggccaaagggccattctcaattttcctcacgagtatcaaatgatgaaggacggtccaaatggcagccacgagaatgcagtggcttcctcgtcgtcgggatatagaggaggaggtggtggtgatgatgggagggaagttattgagttcgagtatttggatgatagtttattggaggagcttttagattatggtgagagatctaaccaagacaattgtaacgacgcaaaccgctagatcatcactacttacttacagtgtaatgtttttggagtaaagagtaataatcaatataatatactttagtttaggaaaaaaaaaaaaaaaaaaa

SEQ ID NO: 4 - protein, 139 aa, Arabidopsis thaliana
  G1791 polypeptide
MERIESYNTNEMKYRGVRKRPWGKYAAEIRDSARHGARVWLGTFNTAEDAARAYDRAAFGMRGQRAILNFPHEYQMMKDGPNGSHENAVASSSSGYRGGGGGDDGREVIEFEYLDDSLLEELLDYGERSNQDNCNDANR

SEQ ID NO: 5 - DNA, 450 bp, Arabidopsis thaliana
  G1795
acaaacacgcaaaaagtcattaatatatggatcaaggaggtcgaggtgtcggtgccgagcatggaaagtaccggggagttcggagacgaccttggggaaaatatgcagcagagatacgagattcgaggaagcacggtgaacgtgtgtggcttggaacgttcgatacggcagaggaagcggctagagcctatgaccaagctgcttactccatgagaggccaagcagcaatccttaacttccctcatgagtataacatggggagtggtgtctcttcttccaccgccatggctggatcttcctccgcctccgcctccgcttcttcttcttctaggcaagtttttgaatttgagtacttggatgatagtgttttggaggagctccttgaggaaggagagaaacctaacaagggcaagaagaaatgagcgagatataattcatgattatttctaa

SEQ ID NO: 6 - protein, 131 aa, Arabidopsis thaliana
  G1795 polypeptide
MDQGGRGVGAEHGKYRGVRRRPWGKYAAEIRDSRKHGERVWLGTFDTAEEAARAYDQAAYSMRGQAAILNFPHEYNMGSGVSSSTAMAGSSSASASASSSSRQVFEFEYLDDSVLEELLEEGEKPNKGKKK

SEQ ID NO: 7 - DNA, 553 bp, Arabidopsis thaliana
  G30
ctcttctgacgcacaacagtatatacacatacacagatatatggatcaaggaggtcgtagcagtggtagtggaggaggaggagccgagcaagggaagtaccgtggagtaaggagacgaccttggggtaaatacgccgcggaaataagagattcgaggaagcacggagagcgtgtgtggctagggacattcgacactgcggaagacgcggctcgagcctatgaccgagccgcctattcaatgagaggcaaagctgccattctcaacttccctcacgagtataacatgggaaccggatcctcatccactgcggctaattcttcttcctcgtcgcagcaagtttttgagtttgagtacttggacgatagcgttttggatgaacttcttgaatatggagagaactataacaagactcataatatcaacatgggcaagaggcaataaagggaatacaatcggtattaactgaaagttatgtgaaagaccattttcagttataacaaataaaataaaatcccaagcgtacaaagctgtttctaaaaaaaaaaaaaaaaa

SEQ ID NO: 8 - protein, 133 aa, Arabidopsis thaliana
  G30 polypeptide
MDQGGRSSGSGGGGAEQGKYRGVRRRPWGKYAAEIRDSRKHGERVWLGTFDTAEDAARAYDRAAYSMRGKAAILNFPHEYNMGTGSSSTAANSSSSSQQVFEFEYLDDSVLDELLEYGENYNKTHNINMGKRQ

SEQ ID NO: 9 - DNA, 579 bp, Oryza sativa
  G3380
ggtccgatccgtaacagtagtagctagttaatttgattattgtccgtccgcggccggtcagtggtcgcaatcgatcgatcgatatcatggacggcgacggcggcggcggatgggacgatcagggcaacggcggcggcgagacgaccaagtaccgtggcgtgcgtcgccggccttctggcaagttcgcggcggagatccgtgactccagcaggcagagcgtccgcgtctggctgggaaccttcgacaccgccgaggaggctgcgcgggcttacgaccgcgccgcctacgccatgcgcggccacctcgccgtcctcaacttccctgctgaggcgcgcaactacgtgcggggatcaggctcgtcgtcctcgtcccgacagcatcagcagcggcaggtgatcgagctggagtgcctagacgaccaagtgctgcaagagatgctcaagggtggcgacgatcagtacaggtcagcagctgggagcaagaggaataactactagctatatatgctgctaacctacttacaatcgcgatacatatcgaggtttggggattttcttctcacctgtgtgcagaggctgc

SEQ ID NO: 10 - protein, 136 aa, Oryza sativa
  G3380 polypeptide
MDGDGGGGWDDQGNGGGETTKYRGVRRRPSGKFAAEIRDSSRQSVRVWLGTFDTAEEAARAYDRAAYAMRGHLAVLNFPAEARNYVRGSGSSSSSRQHQQRQVIELECLDDQVLQEMLKGGDDQYRSAAGSKRNNY

SEQ ID NO: 11 - DNA, 514 bp, Oryza sativa
  G3381
atcgatcatctgctacgaactcaccctatatatatatactccatcttaggagctgcttgatcgatcgacatatatataactaatggatcatcatcatcagcagcagcagcaggagggtgagctggtggccaagtacaggggcgtgcggcggcggccgtggggcaaattcgcggcagagatccgcgactcgagccggcacggcgtccgcgtgtggctgggcaccttcgacacagccgaggaggccgctcgcgcctacgaccgctccgcctactccatgcgcggcgccaacgccgtcctcaacttccccgccgacgcccacatctacgcccgtcaactacacaataataacgccgctgctggctcttcatcttcctcttccgccgccgccgcagcagccaggccgccgccgatcgagttcgagtacctcgatgaccacgtcctgcaggagatgctccgagaccacaccaccaacaagtagcttactactccactatatatgctgcctgctgcttgt

SEQ ID NO: 12 - protein, 131 aa, Oryza sativa
  G3381 polypeptide
MDHHHQQQQQEGELVAKYRGVRRRPWGKFAAEIRDSSRHGVRVWLGTFDTAEEAARAYDRSAYSMRGANAVLNFPADAHIYARQLHNNNAAAGSSSSSSAAAAAARPPPIEFEYLDDHVLQEMLRDHTTNK

SEQ ID NO: 13 - DNA, 375 bp, Oryza sativa
  G3383
atggaggacaaccggagcaaggacacggcgaccaagtaccgcggcgtgaggaggcggccgtggggcaagttcgcggcggagatccgcgacccggagcgcggcggggcgcgcgtctggctcggcaccttcgacaccgccgaggaggcggcgcgtgcctacgaccgcgcggcctacgcccagcgcggcgccgccgccgtgctcaacttcccggccgccgccgccgccggcaggggtggaggagccggcggcgccgcttccgggtcgtcgtcgtcgtcgtccgcgcagcgcggcaggggcgacaagatcgagttcgagtacctcgacgacaaggtgctcgacgatctcctcgacgacgagaagtaccgtggtaaatga

SEQ ID NO: 14 - protein, 124 aa, Oryza sativa
  G3383 polypeptide
MEDNRSKDTATKYRGVRRRPWGKFAAEIRDPERGGARVWLGTFDTAEEAARAYDRAAYAQRGAAAVLNFPAAAAAGRGGGAGGAASGSSSSSSAQRGRGDKIEFEYLDDKVLDDLLDDEKYRGK

SEQ ID NO: 15 - DNA, 466 bp, Oryza sativa
  G3515
gtgtgcgagcggttgcgtccgcatggaggacgacaagagtaaggaggggaaatcgtcgtcgtcgtaccgcggcgtgcggaagcggccgtggggcaagttcgcggcggagatccgcgacccggagcgcgggggagcccgcgtgtggctcggcacgttcgacaccgcggaggaggccgcgcgggcgtacgaccgcgccgcattcgccatgaagggcgccacggccatgctcaacttcccgggagatcatcatcacggcgccgcaagcaggatgaccagcaccggctcttcttcgtcctccttcaccacgcctcctccggcgaactcctccgcggcggcgggccgcggcggctccgatcggacgacggacaaggtggagctggagtgcctcgacgacaaggtcctggaggacctcctcgcggagaccaactatcgtgataagaactactagctagctagctactatggc

SEQ ID NO: 16 - protein, 141 aa, Oryza sativa
  G3515 polypeptide
MEDDKSKEGKSSSSYRGVRKRPWGKFAAEIRDPERGGARVWLGTFDTAEEAARAYDRAAFAMKGATAMLNFPGDHHHGAASRMTSTGSSSSSFTTPPPANSSAAAGRGGSDRTTDKVELECLDDKVLEDLLAETNYRDKNY

SEQ ID NO: 17 - DNA, 393 bp, Zea mays
  G3516
atggaggacgacaagaaggagggcaagtaccgcggcgtgcggaagcggccgtggggcaagttcgccgcggagatccgggacccggagcgcggcggctcccgcgtctggctcggcaccttcgacaccgccgaggaggccgccagggcctacgaccgcgccgcattcgccatgaagggcgccacggccgtgctcaacttccccgccagcggaggatcgtcagctggcgcggctcccggcggccggaccagcggcggctcctcctcgtccaccacgtcggctccggccagcagggggagggcccgtgttcccgactcggagaaggtggagctggagtgcctcgacgacagggtcttggaagagctgctcgcggaagacaagtacaacaagaactaa

SEQ ID NO: 18 - protein, 130 aa, Zea mays
  G3516 polypeptide
MEDDKKEGKYRGVRKRPWGKFAAEIRDPERGGSRVWLGTFDTAEEAARAYDRAAFAMKGATAVLNFPASGGSSAGAAPGGRTSGGSSSSTTSAPASRGRARVPDSEKVELECLDDRVLEELLAEDKYNKN

SEQ ID NO: 19 - DNA, 477 bp, Zea mays
  G3517
tacgtccgatccacagccatcatcgccacccgcgcgcttatggatggcgagtggtccaaggacggcggaggcggcgagccgaccaagtaccgcggcgtgcggcgtcggccctggggcaagtacgcggcggagatccgcgactcgagccggcacggcgtccgcatctggctcggcacgttcgacaccgccgaggaggccgccagggcgtacgaccgctccgccaactccatgcgcggcgccaacgccgtgctcaacttcccggaggacgcgcccgcctacgccgccgccgcctcccgtggctccgccggcggatcctcgtccagaccggcgggctccggccgggacgtgatcgagtttgagtacctcgacgacgaggtgctgcaggagatgctcaggagccaggagccgtcggcggcggcggcgcagaagaagaagtagcgcgagcgccacaggtggcgaaacggccgcttttccaaa

SEQ ID NO: 20 - protein, 132 aa, Zea mays
  G3517 polypeptide
MDGEWSKDGGGGEPTKYRGVRRRPWGKYAAEIRDSSRHGVRIWLGTFDTAEEAARAYDRSANSMRGANAVLNFPEDAPAYAAAASRGSAGGSSSRPAGSGRDVIEFEYLDDEVLQEMLRSQEPSAAAAQKKK

SEQ ID NO: 21 - DNA, 717 bp, Glycine max
  G3518
ctaacacacataacaataacttagcaacattttttccttccttctttctttctttctatactttttgttgttaattctaagttctaagagaagaaaaatggagggtggaagatcatcagtttcaaatgggaatgttgaggttcgttatagagggattagaagaaggccatggggaaagtttgcagcagagattcgtgaccctacaaggaaaggaacaaggatatggcttggaacatttgacactgctgaacaagctgcacgagcttatgatgctgctgcttttcattttcgtggccacagagcaattctcaacttcccaaatgagtatcaatctcataatccaaactcttctttgcctatgcctctagctgtgtcagctcctccttcttattcttcttcttcttccacttctaattattccggtgatgataataataaccaccttgtgagaccagctttttctggagaaataatgcaaggtggtgatcatgatgatgatacttttgagttggagtacttcgataataagttgctcgaggaactccttcagatgcaagataacagacacttctaaaagtaaaatataacacaagccagctatgttgtgttagtcactggcatgaaataaaatgcaaagaaatattgttgattttatttaatatattttgtttgatttttttttttttttttgtagctgatcaaagttcttcgaaatga

SEQ ID NO: 22 - protein, 158 aa, Glycine max
  G3518 polypeptide
MEGGRSSVSNGNVEVRYRGIRRRPWGKFAAEIRDPTRKGTRIWLGTFDTAEQAARAYDAAAFHFRGHRAILNFPNEYQSHNPNSSLPMPLAVSAPPSYSSSSSTSNYSGDDNNNHLVRPAFSGEIMQGGDHDDDTFELEYFDNKLLEELLQMQDNRHF

SEQ ID NO: 23 - DNA, 609 bp, Glycine max
  G3519
tttctttctttctatactttttgtggttctgattattaagttctaagagaataacaatggagggtggaagatcatctgtttcaaatgggaattgtgaggttcggtatagagggattagaagaaggccatggggcaagtttgcagcagagattcgtgaccctacgaggaaagggacaaggatatggcttggaacatttgacactgcggaacaagctgctcgagcttatgatgctgctgcttttcattttcgtggtcatagagcaattctcaacttcccaaatgagtaccaatctcataatccaaactcttctttgcctatgcctctaattgtgcctcctccttcttattcttcttctttcacttctaattattctgctgatgataataaccaccttgtgagacctggagaaataatgcaaggtggtgatcttgatgacacttttgagttggagtacttggataataagttgctcgaggaactccttcagatgcaagataacagacacttctaaaagtaaaatataacacaagccagctatgttgtgttagtcactggcatgaaataaaatgcaaagaaatattgttgattttatttaatatattttgttt

SEQ ID NO: 24 - protein, 151 aa, Glycine max
  G3519 polypeptide
MEGGRSSVSNGNCEVRYRGIRRRPWGKFAAEIRDPTRKGTRIWLGTFDTAEQAARAYDAAAFHFRGHRAILNFPNEYQSHNPNSSLPMPLIVPPPSYSSSFTSNYSADDNNHLVRPGEIMQGGDLDDTFELEYLDNKLLEELLQMQDNRHF

SEQ ID NO: 25 - DNA, 440 bp, Glycine max
  G3520
aaggcacacaatggaagaggagtcaaaggagaaaaagaaggacactaaggaggaaccacgttatagaggagtgcggcggcggccgtgggggaagttcgcggccgagattcgggacccggcccggcacggtgcccgagtgtggctggggacatttctcacggcggaggaggctgctagggcttatgaccgagctgcctatgagatgaggggcgctttagccgttctcaattttccaaatgagtatccttcatgctcttctatgaactcatcttcaacattagcaccttcatcttcttcttcaaattcaatgcttaaaagtgatcatggtaaacaagttattgagttcgagtgcttggatgacaaattgttagaggaccttcttgattgtgatgactatgcctacgagaaagacttgcctaagaactgaacggtttgatcaa

SEQ ID NO: 26 - protein, 138 aa, Glycine max
  G3520 polypeptide
MEEESKEKKKDTKEEPRYRGVRRRPWGKFAAEIRDPARHGARVWLGTFLTAEEAARAYDRAAYEMRGALAVLNFPNEYPSCSSMNSSSTLAPSSSSSNSMLKSDHGKQVIEFECLDDKLLEDLLDCDDYAYEKDLPKN

SEQ ID NO: 27 - DNA, 653 bp, Medicago truncatula
  misc_feature (605)..(605): n is a, c, g, or t
  misc_feature (610)..(610): n is a, c, g, or t
  misc_feature (615)..(615): n is a, c, g, or t
  misc_feature (625)..(625): n is a, c, g, or t
  misc_feature (647)..(647): n is a, c, g, or t
  misc_feature (652)..(652): n is a, c, g, or t
  G3735
ctaatccttcatactaaagaaaacatagacttataacaaaaatattattatttacttcgtatatttttgtgtttcaaattaatggagggagatcataaattagtttcaaattcaacaaatggaaatggaaatggaaatggaaattcagatcaaataaagtatagaggaattcgtagaagaccatggggaaaatttgcagcagaaattcgtgacccaacaaggaaagggacaagaatatggcttggaacatttgatactgctgaacaagctgcaagagcttatgatgctgctgcttttcattttcgtggtcatagagctattctcaatttccctaatgaatatcaagctcctaattcatcttcttcattacctatgcctcttactatgcctccaccaccttcttctaatccacctccttcttcttcttcttcttcctctttttcttcttacaccgttgatgatggttttgatgagcttgaattcttggataataagttgcttcaagaacttcttcaagatggaacacaatagttaactattgaagatcaagtggcatgaaatgtattggtggtcatttaattttctcttcattaatttattttggnttggntatgnatctcatttntatgaataaatgagaatggggnattana

SEQ ID NO: 28 - protein, 149 aa, Medicago truncatula
  G3735 polypeptide
MEGDHKLVSNSTNGNGNGNGNSDQIKYRGIRRRPWGKFAAEIRDPTRKGTRIWLGTFDTAEQAARAYDAAAFHFRGHRAILNFPNEYQAPNSSSSLPMPLTMPPPPSSNPPPSSSSSSSFSSYTVDDGFDELEFLDNKLLQELLQDGTQ

SEQ ID NO: 29 - DNA, 859 bp, Triticum aestivum
  G3736
gcacgaggcttcattctccctcgttccatccaagctccaccatccatcactgatttgcacttacctagctactccgcaacccccacttccggcttcttcatttctcactactagtacgtagttgagattatggagggcggagaaggatccggtggcggcggcgagccgaccaagtaccgcggggtgcgccgcaggccgtggggcaagttcgccgcggagatccgggactcgagccggcacggcgtgcgcatgtggctcggcaccttcgacaccgccgaggaggccgcggccgcctacgaccgctccgcctactccatgcgcggccgcaacgccgtgctcaacttccccgaccgggcgcacgtctacgaggccgaggccaggcgccagggccagggctcttcgtcgtcggcgaggcagcagaatcagcagcagcagcaggggcagagcggggtgatcgagttcgagtacctggacgacgacgtgctgcagtccatgctccacgaccacgacaaatccaacaagtagatcgatggatcatccatccatccatccatggatcgatccataatacctactgtatcatcccggcccggccggcaacatcgacctgcgtgcatgcgcgggcgcggatgcaatctacactacctacctatgcattccggccatatattaggtacgtagattatatgtgtacgagagcctacgagctcgatgaagatcgtacgtggtgcattctgatgcatgaggattccatcgacacgaccctctaccatatatttgatgggtcgatcgagtaatttgcagccagtaatccaatcgatgatatggggttttcaaaaaaaaaaaaaaaaaaaaaaaa

SEQ ID NO: 30 - protein, 131 aa, Triticum aestivum
  G3736 polypeptide
MEGGEGSGGGGEPTKYRGVRRRPWGKFAAEIRDSSRHGVRMWLGTFDTAEEAAAAYDRSAYSMRGRNAVLNFPDRAHVYEAEARRQGQGSSSSARQQNQQQQQGQSGVIEFEYLDDDVLQSMLHDHDKSNK

SEQ ID NO: 31 - DNA, 882 bp, Oryza sativa
  G3737
acacatgcatcgatcattcatggatgccgaattgccgcgatccgggcattatttcgcgccaggagacccaagatcatcgtgtcgcccacgctataaatagctagctagcttgcctttatgttgcatatgccaactgctacatgcaggacgtctgaaactatcattagtgacctgcagcgcctgcagtatatatatacaagtagtagtgagcatggaggacgacaagaaggaggcggcgagcaagtaccgcggcgtacggaggcggccgtggggcaaattcgcggcggagatccgcgacccggagcgcggcggctcacgcgtctggcttggcacgttcgacaccgccgaggaggccgcgcgagcgtacgaccgcgccgcattcgccatgaagggcgctatggccgtgctcaacttcccaggcaggacgagcagcaccggctcttcgtcgtcatcgtcatccacgccgccagctccggtgacgacgagccgccactgcgccgacacgacggagaaggtggagcttgtgtaccttgacgacaaggtgctcgacgagctccttgcggaggactacagctaccgcaacaacaacaactactgatccggccgtcgatgaactgagacggatcgacatggggccggtcgtcggtacgctcgctgaaacgagacccggattgctatcaataagcaagcagaagaaaaccgtctcctatatatagcttcttctgttggcacaagcatatatgggcatgcatgacacatgctactgtgaattgacgggtgtgtgctgtgtgcagactactaaaccacgcttgcaagttgcacgtacgacgtggttgtcaagagcatgcagtccacgaagcagagaaaaacacctggtttatc

SEQ ID NO: 32 - protein, 128 aa, Oryza sativa
  G3737 polypeptide
MEDDKKEAASKYRGVRRRPWGKFAAEIRDPERGGSRVWLGTFDTAEEAARAYDRAAFAMKGAMAVLNFPGRTSSTGSSSSSSSTPPAPVTTSRHCADTTEKVELVYLDDKVLDELLAEDYSYRNNNNY

SEQ ID NO: 33 - DNA, 899 bp, Zea mays
  G3739
cgatataattcactcctctcaacgctcgctgcacacacacaccagtgaacctagccagccatttgccgcatcgatcatcagtcgctgtcacgcgcgccaaaccaaaccaaagcccaaacccagctgcaagtgctactgacagcagctagcaaacacacacccgtcgccatcgctatggacggcgactggtccaaggacggcggaggtggagagccgaccaaatatcgcggcgtgcggcggcggccctggggcaagtacgcggccgagatccgcgactcgagccgccacggcgtccgcatctggctgggcaccttcgacaccgccgaggaggccgccagggcgtacgaccggagcgcctactccatgcgcggcgccaacgccgtcctcaacttcccggaggacgcgcacgcctacgccgccgcctgccgcggctccggatcctcctcatcctcgtccaggcataggcagcagcagcagcagggctccggcagggacgtgatcgagctcgagtacctcgacgacgaggtgctgcaggagatgctcaggaaccacgagccgtcgtcgtctgcgaggaagaagatgtaatgcaagacgactggtacacgtggcgaatgcacgttgcacatcagaatgccatgtatgcgtggggggttacgttcaattgtatgcatgcagtgcagtgactaccggccggctctcctggatatgtcggccatctctctctatatattattaaaatgtcagctcccttctctaatttggcgggagttacatcagtggtactatgcagagttgcatacttgcatatatatgcacattattaattaataactcgatctctcgtggacggtggaacagtgataatcatctcattgtcaattaattttgatcaaagaaat

SEQ ID NO: 34 - protein, 136 aa, Zea mays
  G3739 polypeptide
MDGDWSKDGGGGEPTKYRGVRRRPWGKYAAEIRDSSRHGVRIWLGTFDTAEEAARAYDRSAYSMRGANAVLNFPEDAHAYAAACRGSGSSSSSSRHRQQQQQGSGRDVIELEYLDDEVLQEMLRNHEPSSSARKKM

SEQ ID NO: 35 - DNA, 918 bp, Zea mays
  G3794
attacttgtgcacttgggtgcagtgcctgcagtataatcaagttagggtttaaaagaacctcgaccgcgatcgtatatagatccagattatcattagttattagaccactgtgatatcgatggacgacggcggcgagccaaccaagtaccgcggcgtgcggcgccggccgtcggggaagttcgccgccgagatccgcgactccagccggcagagcgtgcgcatgtggctgggcaccttcgacacggccgaggaggccgcaagggcgtacgaccgcgcggcctacgccatgcgcggccaaatcgccgtgctcaacttccccgccgaggcgcgcaactacgtgcgcggcgggtcgtcgtcgtcccgccagcagcagcagggaggaggaggaggaggaggaagtggcggcggcgccggtcagcaggtgatcgagctggagtgcctggacgatcaggtgctgcaggagatgctcaagggcggcgacgggaaaaaatagttgttagcgtatctgatcacaggtgcacgtgttgaaactgattatgaccaggcgatcgatcccatcttgtgcatgcggcctgccaaagttgctgggtcttctcatcgacctatatatatatgcttctcgatccatatatatatcataaatgcatgcagggtgcatgcatgtaccaagtttggaattataatgctcttggtgctgaattgaagtatactagtatatatagtgtgatccatgtattgaaaaggttgttttgcttaatcgcgtcatgattgcacacgtgcttgtttctgcttaaacaacccatatatatagccggctctggcctttgtcaagtctgcaatccttatacatcgttggtaattcatgcatgagttctatgtaactgcaatttagataaattgtagctaatataatagtc

SEQ ID NO: 36 - protein, 124 aa, Zea mays
  G3794 polypeptide
MDDGGEPTKYRGVRRRPSGKFAAEIRDSSRQSVRMWLGTFDTAEEAARAYDRAAYAMRGQIAVLNFPAEARNYVRGGSSSSRQQQQGGGGGGGSGGGAGQQVIELECLDDQVLQEMLKGGDGKK

SEQ ID NO: 37 - DNA, 859 bp, Arabidopsis thaliana
  G1266
caatccactaacgatccctaaccgaaaacagagtagtcaagaaacagagtattttttctacatggatccatttttaattcagtccccattctccggcttctcaccggaatattctatcggatcttctccagattctttctcatcctcttcttctaacaattactctcttcccttcaacgagaacgactcagaggaaatgtttctctacggtctaatcgagcagtccacgcaacaaacctatattgactcggatagtcaagaccttccgatcaaatccgtaagctcaagaaagtcagagaagtcttacagaggcgtaagacgacggccatgggggaaattcgcggcggagataagagattcgactagaaacggtattagggtttggctcgggacgttcgaaagcgcggaagaggcggctttagcctacgatcaagctgctttctcgatgagagggtcctcggcgattctcaatttttcggcggagagagttcaagagtcgctttcggagattaaatatacctacgaggatggttgttctccggttgtggcgttgaagaggaaacactcgatgagacggagaatgaccaataagaagacgaaagatagtgactttgatcaccgctccgtgaagttagataatgtagttgtctttgaggatttgggagaacagtaccttgaggagcttttggggtcttctgaaaatagtgggacttggtgaaagattaggatttgtattagggaccttaagtttgaagtggttgattaattttaaccctaatatgttttttgtttgcttaaatatttgattctattgagaaacatcgaaaacagtttgtatgtacttttgtgatacttggcg

SEQ ID NO: 38 - protein, 218 aa, Arabidopsis thaliana
  G1266 polypeptide
MDPFLIQSPFSGFSPEYSIGSSPDSFSSSSSNNYSLPFNENDSEEMFLYGLIEQSTQQTYIDSDSQDLPIKSVSSRKSEKSYRGVRRRPWGKFAAEIRDSTRNGIRVWLGTFESAEEAALAYDQAAFSMRGSSAILNFSAERVQESLSEIKYTYEDGCSPVVALKRKHSMRRRMTNKKTKDSDFDHRSVKLDNVVVFEDLGEQYLEELLGSSENSGTW

SEQ ID NO: 39 - DNA, 1262 bp, Arabidopsis thaliana
  G45
attaatactctgcatctagtccttttcaagagtacacaatctgcacttttttaatgaaaatagtacacaatctttatacttcaaactgaggtaacattattaaattaatttattgaagttgacttaagatgatctattcacataatggtacgtgtgtgtgtgtatacacagaaaacccctgattttatgtggaacctaaaaccctccatgaaatgcggtcagtaccttagaacacaagtttcaccaactgtacttcccaattatcctgccgcagattcaacaatggcttttggcaatatccaagaactagacggcgagatcctaaagaacgtttgggcgaattacatcggaacaccacaaaccgatacaagatcaattcaagttccagaagtttctagaacttgggaagcgttgcctacccttgatgacataccagaaggttctagagaaatgcttcaaagcctagatatgtcgacggaggaccaggaatggacagagattctcgatgctattgcttctttcccaaacaaaaccaatcatgatccattaaccaaccctaccattgattcatgttctttgtcttctcgggtttcttgcaaaacaagaaaatacaggggagtacggaagcgtccgtgggggaaatttgcagccgaaatcagggattcgacgagaaacggtgttagggtttggctcggaacgttccaaactgcagaggaagcagctatggcttacgataaagccgcggttagaattagaggtactcaaaaagctcacacaaattttcagctcgaaacagttataaaagctatggaaatggattgcaacccaaactactaccggatgaacaactcaaatacgtccgatccattaagaagcagccgcaaaatcggattgagaacaggaaaagaggcggttaaggcttatgatgaagtcgttgatgggatggttgaaaaccattgtgcccttagctattgttcaactaaggagcactcggagactcgtggtttgcgtgggagtgaagaaacttggttcgatttaagaaagagacgaaggagtaatgaagattctatgtgtcaagaagttgaaatgcagaagacggttactggagaagagacagtatgtgatgtgtttggtttgtttgagtttgaggatttgggaagtgattatttggagacgttattatcttctttttgacagaaatacattgaaaactaccgttgctaatttgataggtatacatatatagacatgtatatattgta

SEQ ID NO: 40 - protein, 349 aa, Arabidopsis thaliana
  G45 polypeptide
MVRVCVYTQKTPDFMWNLKPSMKCGQYLRTQVSPTVLPNYPAADSTMAFGNIQELDGEILKNVWANYIGTPQTDTRSIQVPEVSRTWEALPTLDDIPEGSREMLQSLDMSTEDQEWTEILDAIASFPNKTNHDPLTNPTIDSCSLSSRVSCKTRKYRGVRKRPWGKFAAEIRDSTRNGVRVWLGTFQTAEEAAMAYDKAAVRIRGTQKAHTNFQLETVIKAMEMDCNPNYYRMNNSNTSDPLRSSRKIGLRTGKEAVKAYDEVVDGMVENHCALSYCSTKEHSETRGLRGSEETWFDLRKRRRSNEDSMCQEVEMQKTVTGEETVCDVFGLFEFEDLGSDYLETLLSSF

SEQ ID NO: 41 - DNA, 933 bp, Arabidopsis thaliana
  G1752
aaaaaaaaaaaaaaaaaaaaacttatggaatattcccaatcttccatgtattcatctccaagttcttggagctcatcacaagaatcactcttatggaacgagagctgtttcttggatcaatcatctgaacctcaagccttcttttgccctaattatgattactccgatgactttttctcatttgagtcaccggagatgatgattaaggaagaaattcaaaacggcgacgtttctaactccgaagaagaagaaaaggttggaattgatgaagaaagatcatacagaggagtgaggaaaaggccgtgggggaaatttgcagcggagataagagattcaacgaggaatggaattagggtttggctcgggacatttgacaaagccgaggaagccgctcttgcttatgatcaagcggctttcgccacaaaaggatctcttgcaacacttaatttcccggtggaagtggttagagagtcgctaaagaaaatggagaatgtgaatcttcatgatggaggatctccggttatggccttgaagagaaaacattctcttcgaaaccggcctagagggaaaaagcgatcctcttcttcttcttcttcttcttctaattcttcttcttgctcttcttcttcgtctacttcttcaacatcaagaagtagtagtaagcagagtgttgtgaagcaagaaagtggtacacttgtggtttttgaagatttaggtgctgagtatttagaacaacttcttatgagctcatgttgatcttgtaattgatttcagcaaaagccactattaaactttaattttgtgataattaatcttgaaatttgttttgttcattctgcaatttctttggttctcttattttttgtttgttgtatccaaatgaaattattggaagagatggtgatgttaaagtgtatatatataaaaaaaaaa

SEQ ID NO: 42 - protein, 243 aa, Arabidopsis thaliana
  G1752 polypeptide
MEYSQSSMYSSPSSWSSSQESLLWNESCFLDQSSEPQAFFCPNYDYSDDFFSFESPEMMIKEEIQNGDVSNSEEEEKVGIDEERSYRGVRKRPWGKFAAEIRDSTRNGIRVWLGTFDKAEEAALAYDQAAFATKGSLATLNFPVEVVRESLKKMENVNLHDGGSPVMALKRKHSLRNRPRGKKRSSSSSSSSSNSSSCSSSSSTSSTSRSSSKQSVVKQESGTLVVFEDLGAEYLEQLLMSSC

SEQ ID NO: 43 - DNA, 832 bp, Arabidopsis thaliana
  G2512
aacttagtgccacttagacacaataagaaaaccgttaacaagaagaaaaaaaaaagatcgaaaatggaatatcaaactaacttcttaagtggagagttttccccggagaactcttcttcaagctcatggagctcacaagaatcattcttgtgggaagagagtttcttacatcaatcatttgaccaatccttccttttatctagccctactgataactactgtgatgacttctttgcatttgaatcatcaatcataaaagaagaaggaaaagaagccaccgtggcggccgaggaggaggagaagtcatacagaggagtgaggaaacggccgtgggggaaattcgcggccgagataagagactcaacgaggaaagggataagagtgtggcttgggacattcgacaccgcggaggcggcggctctcgcttatgatcaggcggctttcgctttgaaaggcagcctcgcagtactcaatttccccgcggatgtcgttgaagaatctctccggaagatggagaatgtgaatctcaatgatggagagtctccggtgatagccttgaagagaaaacactccatgagaaaccgtcctagaggaaagaagaaatcttcttcttcttcgacgttgacatcttctccttcttcctcctcctcctattcatcttcttcgtcttcttcttctttgtcgtcaagaagtagaaaacagagtgttgttatgacgcaagaaagtaatacaacacttgtggttcttgaggatttaggtgctgaatacttagaagagcttatgagatcatgttcttgataatctctgcttctacaatttttatgtaatttga

SEQ ID NO: 44 - protein, 244 aa, Arabidopsis thaliana
  G2512 polypeptide
MEYQTNFLSGEFSPENSSSSSWSSQESFLWEESFLHQSFDQSFLLSSPTDNYCDDFFAFESSIIKEEGKEATVAAEEEEKSYRGVRKRPWGKFAAEIRDSTRKGIRVWLGTFDTAEAAALAYDQAAFALKGSLAVLNFPADVVEESLRKMENVNLNDGESPVIALKRKHSMRNRPRGKKKSSSSSTLTSSPSSSSSYSSSSSSSSLSSRSRKQSVVMTQESNTTLVVLEDLGAEYLEELMRSCS

SEQ ID NO: 45 - DNA, 913 bp, Arabidopsis thaliana
  G1006
gataaatcaatcaacaaaacaaaaaaaactctatagttagtttctctgaaaatgtacggacagtgcaatatagaatccgactacgctttgttggagtcgataacacgtcacttgctaggaggaggaggagagaacgagctgcgactcaatgagtcaacaccgagttcgtgtttcacagagagttggggaggtttgccattgaaagagaatgattcagaggacatgttggtgtacggactcctcaaagatgccttccattttgacacgtcatcatcggacttgagctgtctttttgattttccggcggttaaagtcgagccaactgagaactttacggcgatggaggagaaaccaaagaaagcgataccggttacggagacggcagtgaaggcgaagcattacagaggagtgaggcagagaccgtgggggaaattcgcggcggagatacgtgatccggcgaagaatggagctagggtttggttagggacgtttgagacggcggaagatgcggctttagcttacgatatagctgcttttaggatgcgtggttcccgcgctttattgaattttccgttgagggttaattccggtgaacctgacccggttcggatcacgtctaagagatcttcttcgtcgtcgtcgtcgtcgtcctcttctacgtcgtcgtctgaaaacgggaagttgaaacgaaggagaaaagcagagaatctgacgtcggaggtggtgcaggtgaagtgtgaggttggtgatgagacacgtgttgatgagttattggtttcataagtttgatcttgtgtgttttgtagttgaatagttttgctataaatgttgaggcaccaagtaaaagtgttcccgtgatgtaaattagttactaaacagagccatatatcttcaatcaaaaaaaaaaaaaaaa

SEQ ID NO: 46 - protein, 243 aa, Arabidopsis thaliana
  G1006 polypeptide
MYGQCNIESDYALLESITRHLLGGGGENELRLNESTPSSCFTESWGGLPLKENDSEDMLVYGLLKDAFHFDTSSSDLSCLFDFPAVKVEPTENFTAMEEKPKKAIPVTETAVKAKHYRGVRQRPWGKFAAEIRDPAKNGARVWLGTFETAEDAALAYDIAAFRMRGSRALLNFPLRVNSGEPDPVRITSKRSSSSSSSSSSSTSSSENGKLKRRRKAENLTSEVVQVKCEVGDETRVDELLVS

SEQ ID NO: 47 - DNA, 964 bp, Arabidopsis thaliana
  G28
gaaatctcaacaagaaccaaaccaaacaacaaaaaaacattcttaataattatctttctgttatgtcgatgacggcggattctcaatctgattatgcttttcttgagtccatacgacgacacttactaggagaatcggagccgatactcagtgagtcgacagcgagttcggttactcaatcttgtgtaaccggtcagagcattaaaccggtgtacggacgaaaccctagctttagcaaactgtatccttgcttcaccgagagctggggagatttgccgttgaaagaaaacgattctgaggatatgttagtttacggtatcctcaacgacgcctttcacggcggttgggagccgtcttcttcgtcttccgacgaagatcgtagctctttcccgagtgttaagatcgagactccggagagtttcgcggcggtggattctgttccggtcaagaaggagaagacgagtcctgtttcggcggcggtgacggcggcgaagggaaagcattatagaggagtgagacaaaggccgtgggggaaatttgcggcggagattagagatccggcgaagaacggagctagggtttggttaggaacgtttgagacggcggaggacgcggcgttggcttacgacagagctgctttcaggatgcgtggttcccgcgctttgttgaattttccgttgagagttaattcaggagaacccgacccggttcgaatcaagtccaagagatcttctttttcttcttctaacgagaacggagctccgaagaagaggagaacggtggccgccggtggtggaatggataagggattgacggtgaagtgcgaggttgttgaagtggcacgtggcgatcgtttattggttttataattttgatttttctttgttggatgattatatgattcttcaaaaaagaagaacgttaataaaaaaattcgtttattattaaaaaaaaaaaaaaaaaa

SEQ ID NO: 48 - protein, 268 aa, Arabidopsis thaliana
  G28 polypeptide
MSMTADSQSDYAFLESIRRHLLGESEPILSESTASSVTQSCVTGQSIKPVYGRNPSFSKLYPCFTESWGDLPLKENDSEDMLVYGILNDAFHGGWEPSSSSSDEDRSSFPSVKIETPESFAAVDSVPVKKEKTSPVSAAVTAAKGKHYRGVRQRPWGKFAAEIRDPAKNGARVWLGTFETAEDAALAYDRAAFRMRGSRALLNFPLRVNSGEPDPVRIKSKRSSFSSSNENGAPKKRRTVAAGGGMDKGLTVKCEVVEVARGDRLLVL

SEQ ID NO: 49 - DNA, 913 bp, Arabidopsis thaliana
  G22
agaaaacatctctcactctctaaaatacacactctcatcaaaaaccttctcttcggttcagaagcattcaagaatccattatgagctcatctgattccgttaataacggcgttaactcacggatgtacttccgtaacccgagtttcagcaacgttatcttaaacgataactggagcgacttgccgttaagtgtcgacgattctcaagacatggctatttacaacactctccgtgatgccgttagctccggctggacaccctccgttcctcccgttacctctccggcggaggaaaataagcctccggcgacgaaggcgagtggctcacacgcgccgaggcagaaggggatgcagtacagaggagtgaggaggaggccgtgggggaaattcgcggcggagattagggatccgaagaagaacggagctagggtttggctcgggacttacgagacgccggaggacgcggcggtggcgtacgaccgagcggcgtttcagctcagaggatcgaaagctaagctgaattttccgcatttgattggttcttgtaagtatgagccggttaggattaggcctcgccgtcgctcgccggaaccgtcagtctccgatcagttaacgtcggagcagaagagggaaagccacgtggatgacggcgagtctagtttggttgtaccggagttggatttcacggtggatcagttttacttcgatggtagtttattaatggaccaatcagaatgttcttattctgataatcggatataattagttttaagattaagcaaaatttgtccaacgagttttgctgtatgaaatatctatcgatgactcaacaggttttgatcatgatcatatgtaatgtgatggaaattaaatattgacgtttgtttttttgttgtaaaaaaaaaaaaaaaaaa

SEQ ID NO: 50 - protein, 226 aa, Arabidopsis thaliana
  G22 polypeptide
MSSSDSVNNGVNSRMYFRNPSFSNVILNDNWSDLPLSVDDSQDMAIYNTLRDAVSSGWTPSVPPVTSPAEENKPPATKASGSHAPRQKGMQYRGVRRRPWGKFAAEIRDPKKNGARVWLGTYETPEDAAVAYDRAAFQLRGSKAKLNFPHLIGSCKYEPVRIRPRRRSPEPSVSDQLTSEQKRESHVDDGESSLVVPELDFTVDQFYFDGSLLMDQSECSYSDNRI

SEQ ID NO: 51 - DNA, 1084 bp, Arabidopsis thaliana
  G26
ttggcttgtacccaaacccatctttgacttcaaaaataaaataaaaataatcataattgacatcatcggataatgcatagcgggaagagacctctatcaccagaatcaatggccggaaatagagaagagaaaaaagagttgtgttgttgctcaactttgtcggaatctgatgtgtctgattttgtctctgaactcactggtcaacccatcccatcatccattgatgatcaatcttcgtcgcttactcttcaagaaaaaagtaactcgaggcaacgaaactacagaggcgtgaggcaaagaccgtggggaaaatgggcggctgagattcgtgacccgaacaaggcagctcgtgtgtggcttgggacgttcgacactgcagaagaagccgccttagcgtatgataaagctgcatttgagtttagaggtcacaaggccaagcttaacttccccgagcatattcgtgtcaaccctactcaactctatccatcgcccgctacttcccatgatcgcattatcgtgacaccacctagtccacctccaccaattgctcctgacatacttcttgatcaatatggccactttcaatctcgaagtagtgattccagtgccaacttgtccatgaatatgctgtcttcttcgtcttcatctttgaatcatcaagggctaagaccaaatttggaggatggtgaaaacgtgaagaacattagtatccacaaacgacgaaaataacatgttaatggcataaatatctcttcgtccaagttatcaaacgcattgacctccggctttgatcattttaggcgcttaatctctttacgacttcattttggtagtctttaaagagtctatggagtggatttagctaggaatcaggccttatggatgaaaaatatataaattttgaacatgactatgcaagaatgggatgaagactacttagcttggaaaacgtcctgataggtcatgacgactatatccacagaagatgaccgacggagacaacaacatgcctcacctgatcgaccgatcaaatgagataatgtgttgaccggaccggtcggatcaggttgggtcgagtatatca

SEQ ID NO: 52 - protein, 218 aa, Arabidopsis thaliana
  G26 polypeptide
MHSGKRPLSPESMAGNREEKKELCCCSTLSESDVSDFVSELTGQPIPSSIDDQSSSLTLQEKSNSRQRNYRGVRQRPWGKWAAEIRDPNKAARVWLGTFDTAEEAALAYDKAAFEFRGHKAKLNFPEHIRVNPTQLYPSPATSHDRIIVTPPSPPPPIAPDILLDQYGHFQSRSSDSSANLSMNMLSSSSSSLNHQGLRPNLEDGENVKNISIHKRRK

SEQ ID NO: 53 - DNA, 1123 bp, Arabidopsis thaliana
  G1751
aaacacaaacaaaactcatattttcaatctccaggtgctttacaccaacagagtcgcaagaaaacaaaaaccaaactcggatttagtttgacagaagaaggaatcgagagtcgggtatgcattatcctaacaacagaaccgaattcgtcggagctccagccccaacccggtatcaaaaggagcagttgtcaccggagcaagagctttcagttattgtctctgctttgcaacacgtgatctcaggggaaaacgaaacggcgccgtgtcagggtttttccagtgacagcacagtgataagcgcgggaatgcctcggttggattcagacacttgtcaagtctgtaggatcgaaggatgtctcggctgtaactactttttcgcgccaaatcagagaattgaaaagaatcatcaacaagaagaagagattactagtagtagtaacagaagaagagagagctctcccgtggcgaagaaagcggaaggtggcgggaaaatcaggaagaggaagaacaagaagaatggttacagaggagttaggcaaagaccttggggaaaatttgcagctgagatcagagatcctaaaagagccacacgtgtttggcttggtactttcgaaaccgccgaagatgcggctcgagcttatgatcgagccgcgattggattccgtgggccaagggctaaactcaacttcccctttgtggattacacgtcttcagtttcatctcctgttgctgctgatgatataggagcaaaggcaagtgcaagcgccagtgtgagcgccacagattcagttgaagcagagcaatggaacggaggaggaggggattgcaatatggaggagtggatgaatatgatgatgatgatggattttgggaatggagattcttcagattcaggaaatacaattgctgatatgttccagtgataaatgagctctttcttgttggcgttttttggagttaagtgcaagaagagattgacactgtggcttgtttaaagtgaacaagaacaagaaagcatgtaattagtagtctcattcttttgtttgtggtcaattctatgtttatctcatataaaatctgagttaaacctatctgaggagagagtaaataaagaggttaagaa

SEQ ID NO: 54 - protein, 268 aa, Arabidopsis thaliana
  G1751 polypeptide
MHYPNNRTEFVGAPAPTRYQKEQLSPEQELSVIVSALQHVISGENETAPCQGFSSDSTVISAGMPRLDSDTCQVCRIEGCLGCNYFFAPNQRIEKNHQQEEEITSSSNRRRESSPVAKKAEGGGKIRKRKNKKNGYRGVRQRPWGKFAAEIRDPKRATRVWLGTFETAEDAARAYDRAAIGFRGPRAKLNFPFVDYTSSVSSPVAADDIGAKASASASVSATDSVEAEQWNGGGGDCNMEEWMNMMMMMDFGNGDSSDSGNTIADMFQ

SEQ ID NO: 55 - DNA, 1200 bp, Arabidopsis thaliana
  G589
aaaaaaacagaagccatgaactcctcgtctcttctaactccttcatcatctccttctccacatcttcaatctcctgcaacattcgaccacgatgatttcctccaccacatcttctcctccactccttggccctcatccgttctcgacgacactcctccaccaacttccgattgtgcccccgtcactggattccaccaccacgacgccgattcaagaaaccagatcactatgattcctttgtcacataaccatcctaatgacgctctcttcaatggcttctccaccggatctctccctttccacctccctcaaggatcgggaggtcaaacgcaaacgcagtcgcaggcgacggcgtcagccaccaccggtggtgcaacggcgcaacctcagacaaagcctaaagtccgagctaggagaggtcaagccactgatcctcacagtatcgccgaacggttacggagagagaggatagcggaaagaatgaaatctcttcaagaacttgtccctaatggtaacaagacagacaaagcatcaatgctcgatgagattatcgattatgtcaagttcttacagctccaagtcaaggtactaagcatgagtagactgggcggtgctgcttctgcttcttctcaaatctctgaggatgccggtggatcccacgaaaacacctcctcctccggcgaggcgaagatgacggagcaccaagttgcaaagctaatggaagaggacatgggatcagccatgcaatatctacaaggcaaaggtctttgcctcatgcccatctcgttagccaccaccatctccaccgccacgtgtccttctcgtagccccttcgttaaagataccggcgttcctttgtctcctaacctatccactacaatagttgctaacggtaatggctcatcgttggtcaccgttaaagacgctccctccgtttccaagccgtgataacggccatttgtccatttcattttcccttttttgggtgggaaagagagaaaaaagtttagaagacaaagacaagtgggataggtggttttggtcaaagtttagaaagaataaggtcgtgttttcggatacgacaccgtatttgcgtacactttggttttctgtctttacctactacaaaccacccataagcacactcatgttatcatgtttttttttttttggtttataaagatataaaaaaaaaaaaaa

SEQ ID NO: 56 - protein, 310 aa, Arabidopsis thaliana
  G589 polypeptide
MNSSSLLTPSSSPSPHLQSPATFDHDDFLHHIFSSTPWPSSVLDDTPPPTSDCAPVTGFHHHDADSRNQITMIPLSHNHPNDALFNGFSTGSLPFHLPQGSGGQTQTQSQATASATTGGATAQPQTKPKVRARRGQATDPHSIAERLRRERIAERMKSLQELVPNGNKTDKASMLDEIIDYVKFLQLQVKVLSMSRLGGAASASSQISEDAGGSHENTSSSGEAKMTEHQVAKLMEEDMGSAMQYLQGKGLCLMPISLATTISTATCPSRSPFVKDTGVPLSPNLSTTIVANGNGSSLVTVKDAPSVSKP

SEQ ID NO: 57 - DNA, 1023 bp, Arabidopsis thaliana
  G6
tatctatccgagaatggccaagatgggcttgaaacccgacccggctactactaaccagacccacaataatgccaaggagattcgttacagaggcgttaggaagcgtccttggggccgttatgccgccgagatccgagatccgggcaagaaaacccgcgtctggcttggcactttcgatacggctgaagaggcggcgcgtgcttacgatacggcggcgcgtgattttcgtggtgctaaggctaagaccaatttcccaacttttctcgagctgagtgaccagaaggtccctaccggtttcgcgcgtagccctagccagagcagcacgctcgactgtgcttctcctccgacgttagttgtgccttcagcgacggctgggaatgttcccccgcagctcgagcttagtctcggcggaggaggcggcggctcgtgttatcagatcccgatgtcgcgtcctgtctactttttggacctgatggggatcggtaacgtaggtcgtggtcagcctcctcctgtgacatcggcgtttagatcgccggtggtgcatgttgcgacgaagatggcttgtggtgcccaaagcgactctgattcgtcatcggtcgttgatttcgaaggtgggatggagaagagatctcagctgttagatctagatcttaatttgcctcctccatcggaacaggcctgagcttttaacggtgtcgtttcaattcgaagcgcatgcgtttcttcttctttttgagctgtgaaaattcgttttctcatagtttttcctctctctctctctcagtctaaatttattaccagtttttagaaagaaaaaacagattaaatctgagagagaaaaatataattttagctgacatggatcgttatgtacatattattacataaccggagatctgaacttttgttgtgtgcttttaattttttgcgacttggtttcaccccatgttgtttctctattttttttactactttttttttttttgttcttccaaattttcaatcaataatttggtaatcttc

SEQ ID NO: 58 - protein, 222 aa, Arabidopsis thaliana
  G6 polypeptide
MAKMGLKPDPATTNQTHNNAKEIRYRGVRKRPWGRYAAEIRDPGKKTRVWLGTFDTAEEAARAYDTAARDFRGAKAKTNFPTFLELSDQKVPTGFARSPSQSSTLDCASPPTLVVPSATAGNVPPQLELSLGGGGGGSCYQIPMSRPVYFLDLMGIGNVGRGQPPPVTSAFRSPVVHVATKMACGAQSDSDSSSVVDFEGGMEKRSQLLDLDLNLPPPSEQA

SEQ ID NO: 59 - DNA, 1059 bp, Arabidopsis thaliana
  G1004
atggcgactcctaacgaagtatctgcactttggttcatcgagaaacatctactcgacgaggcttctcctgtggctacagatccatggatgaagcacgaatcatcatcagcaacagaatctagctctgactcttcttctatcatcttcggatcatcgtcctcttctttcgccccaattgatttctctgaatccgtatgcaaacctgaaatcatcgatctcgatactcccagatctatggaatttctatcgattccatttgaatttgactcagaagtttctgtttctgatttcgattttaaaccttctaatcaaaatcaaaatcagtttgaaccggagcttaaatctcaaattcgtaaaccgccattgaagatttcgcttccagctaaaacagagtggattcaattcgcagctgaaaacaccaaaccggaagttactaaaccggtttcggaagaagagaagaagcattacagaggagtaagacaaagaccgtgggggaaattcgcggcggagattcgtgacccgaataaacgcggatctcgcgtttggcttgggacgtttgatacagcgattgaagcggctagagcttatgacgaagcagcgtttagactacgaggatcgaaagcgattttgaatttccctcttgaagttgggaagtggaaaccacgcgccgatgaaggtgagaagaaacggaagagagacgatgatgagaaagtgactgtggttgagaaagtgttgaagacggaacagagcgttgacgttaacggtggagagacgtttccgtttgtaacgtcgaatttaacggaattatgtgactgggatttaacggggtttcttaactttccgcttctgtcgccgttatctcctcatccaccgtttggttattcccagttgaccgttgtttgattagttttttttgagtttttgaacgatgtgtatgctgacgtggacgtacacgtaggtgcatgcgatgaaaaaaacatctatttgttcatatttttgcgtttttctatttgttcattctttttcacaattcacaatacattatttcagttaatgatc

SEQ ID NO: 60 - protein, 300 aa, Arabidopsis thaliana
  G1004 polypeptide
MATPNEVSALWFIEKHLLDEASPVATDPWMKHESSSATESSSDSSSIIFGSSSSSFAPIDFSESVCKPEIIDLDTPRSMEFLSIPFEFDSEVSVSDFDFKPSNQNQNQFEPELKSQIRKPPLKISLPAKTEWIQFAAENTKPEVTKPVSEEEKKHYRGVRQRPWGKFAAEIRDPNKRGSRVWLGTFDTAIEAARAYDEAAFRLRGSKAILNFPLEVGKWKPRADEGEKKRKRDDDEKVTVVEKVLKTEQSVDVNGGETFPFVTSNLTELCDWDLTGFLNFPLLSPLSPHPPFGYSQLTVV

SEQ ID NO: 61 - DNA, 1281 bp, Arabidopsis thaliana
  G1005
gctttttgtgttgaagagagagtttcctatcttctccattcctcccaccatctccctcatcttcatcttcctctctctttctctctttctcaacaatctctattagatctttctccattaccattacctctggctttctcttaaatccaccatcatgaggagaggaagaggctcttccgccgtcgccggacctaccgtcgttgccgccatcaacggatctgtaaaagaaatcagattcagaggcgtaaggaagagaccttggggacgattcgcagctgagatccgtgatccatggaaaaaagctcgtgtttggttaggtactttcgattccgccgaagaagctgctcgcgcttacgactccgccgctcgtaacctccgtggtcctaaagccaaaactaatttccccatcgattcttcttctcctcctcctcctaatctccgatttaatcagattcgtaatcaaaatcaaaaccaagtcgatccgtttatggaccaccggttattcaccgaccatcaacaacagttcccgattgttaaccggcctactagtagcagcatgagcagcaccgttgaatcgtttagcggacccagacctacgacgatgaaaccggccacgacgaagagatatcctagaactccaccggttgttccggaggattgtcacagcgattgcgattcgtcgtcgtctgtaatcgacgacgacgacgatatcgcatcgtcttcacggcgacggaatccgccgtttcaattcgatcttaattttccaccgttggattgtgttgacttgttcaatggcgctgatgatcttcactgtaccgatctacgtctctaatgaattggtaaaatcaaactcaaaatcacagatccgtgatcggtttgattttaatcgaaaacacacaacaaaatccttttttttttttttttaaattttctgtttcgttgatctcatataatttttactatgcgggagaaatagaaagacaaagaaacgaagaagaagaagaagatggtgatgagcttgagagagcttgagctggttctgtgtttcttctgtgatgatattgtaagagtattattattttactattattactaaatcttcaaaaccaagaagaagaagaccgaacacgatgatctgttgtgtctgtttgttttactgtaagaaaaacgcagatctgggtttcgtttttttcttgagatagatcaaacaacccccatctttgtaacatatacatttggaacactcatgattctaaataaaaaatctagaatctttttttc

SEQ ID NO: 62 - protein, 225 aa, Arabidopsis thaliana
  G1005 polypeptide
MRRGRGSSAVAGPTVVAAINGSVKEIRFRGVRKRPWGRFAAEIRDPWKKARVWLGTFDSAEEAARAYDSAARNLRGPKAKTNFPIDSSSPPPPNLRFNQIRNQNQNQVDPFMDHRLFTDHQQQFPIVNRPTSSSMSSTVESFSGPRPTTMKPATTKRYPRTPPVVPEDCHSDCDSSSSVIDDDDDIASSSRRRNPPFQFDLNFPPLDCVDLFNGADDLHCTDLRL

SEQ ID NO: 63 - protein, 14 aa, Arabidopsis thaliana
  misc_feature (2)..(5): Xaa can be any naturally occurring amino acid
  misc_feature (7)..(9): Xaa can be any naturally occurring amino acid
  misc_feature (11)..(13): Xaa can be any naturally occurring amino acid
  EDLL Domain
EXXXXDXXXLXXXL

SEQ ID NO: 64 - DNA, 333 bp, Artificial sequence
  Artificial sequence
  P5381 LexAOP and polylinker sequence
acatatccatatctaatcttacctcgactgctgtatataaaaccagtggttatatgtccagtactgctgtatataaaaccagtggttatatgtacagtacgtcgatcgatcgacgactgctgtatataaaaccagtggttatatgtacagtactgctgtatataaaaccagtggttatatgtacagtacgtcgaggggatgatcaagacccttcctctatataaggaagttcatttcatttggagaggacacgctgacaagctgactctagcagatctggtaccgtcgacggtgagctccgcggccgctctagacaggcctcgtaccggatcc

SEQ ID NO: 65 - DNA, 406 bp, Artificial sequence
  Artificial sequence
  P21195 GAL4 and polylinker sequence
agatctatgcccaattttaatcaaagtgggaatattgctgatagctcattgtccttcactttcactaacagtagcaacggtccgaacctcataacaactcaaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatataactatctattcgatgatgaagataccccaccaaacccaaaaaaagagggtaccgtcgacggtgagctccgcggccgctctagacaggcctcgtaccggatcc

SEQ ID NO: 66 - DNA, 411 bp, Artificial sequence
  Artificial sequence
  P21378 GAL4 and polylinker
agatctggtaccgtcgacggtgagctccgcggccgccccaattttaatcaaagtgggaatattgctgatagctcattgtccttcactttcactaacagtagcaacggtccgaacctcataacaactcaaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatataactatctattcgatgatgaagataccccaccaaacccaaaaaaagagtagtaagctctagacaggcctcgtaccggatcc

SEQ ID NO: 67 - DNA, 3523 bp, Artificial sequence
  Artificial sequence
  misc_feature (1)..(4): n is a, c, g, or t
  P5375 pMEN48 insert
nnnnaagctttgagctccgcggccgcaagacccttcctctatataaggaagttcatttcatttggagaggacacgctcgagtataagagctcatttttacaacaattaccaacaacaacaaacaacaaacaacattacaattacatttacaattaccatggaagcgttaacggccaggcaacaagaggtgtttgatctcatccgtgatcacatcagccagacaggtatgccgccgacgcgtgcggaaatcgcgcagcgtttggggttccgttccccaaacgcggctgaagaacatctgaaggcgctggcacgcaaaggcgttattgaaattgtttccggcgcatcacgcgggattcgtctgttgcaggaagaggaagaagggttgccgctggtaggtcgtgtggctgccggtgaaccacttctggcgcaacagcatattgaaggtcattatcaggtcgatccttccttattcaagccgaatgctgatttcctgctgcgcgtcagcgggatgtcgatgaaagatatcggcattatggatggtgacttgctggcagtgcataaaactcaggatgtacgtaacggtcaggtcgttgtcgcacgtattgatgacgaagttaccgttaagcgcctgaaaaaacagggcaataaagtcgaactgttgccagaaaatagcgagtttaaaccaattgtcgtagatcttcgtcagcagagcttcaccattgaagggctggcggttggggttattcgcaacggcgactggctggaattccccaattttaatcaaagtgggaatattgctgatagctcattgtccttcactttcactaacagtagcaacggtccgaacctcataacaactcaaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatataactatctattcgatgatgaagataccccaccaaacccaaaaaaagagtagctagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaatctagaacatatccatatctaatcttacctcgactgctgtatataaaaccagtggttatatgtccagtactgctgtatataaaaccagtggttatatgtacagtacgtcgatcgatcgacgactgctgtatataaaaccagtggttatatgtacagtactgctgtatataaaaccagtggttatatgtacagtacgtcgaggggatgatcaagacccttcctctatataaggaagttcatttcatttggagaggacacgctcgagtataagagctcatttttacaacaattaccaacaacaacaaacaacaaacaacattacaattacatttacaattaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggagggatcctctagctagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaatcgacctgcagccactcgaagcggccggccgccactcgaga

SEQ ID NO: 68 - DNA, 3158 bp, Artificial sequence
  Artificial sequence
  misc_feature (7)..(10): n is a, c, g, or t
  misc_feature (17)..(52): n is a, c, g, or t
  pMEN065 overexpression vector
aagcttnnnnctgcagnnnnnnnnnnnnnnnnnnnnnnnnnnnnnnnnnnnntcggattccattgcccagctatctgtcactttattgtgaagatagtgaaaaagaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatggtccgattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgacaagctgactctagcagatctggtaccgtcgacggtgagctccgcggccgctctagacaggcctcgtaccggatcctctagctagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaatcgacctgcagccactcgaagcggccggccgccactcgagatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgggatctctgcggaacaggcggtcgaaggtgccgatatcattacgacagcaacggccgacaagcacaacgccacgatcctgagcgacaatatgatcgggcccggcgtccacatcaacggcgtcggcggcgactgcccaggcaagaccgagatgcaccgcgatatcttgctgcgttcggatattttcgtggagttcccgccacagacccggatgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggctcgaga

SEQ ID NO: 69 - DNA, 574 bp, Cauliflower mosaic virus
  CaMV 35S promoter
gcggattccattgcccagctatctgtcactttattgtgaagatagtgaaaaagaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatggtccgattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctga

SEQ ID NO: 70 - DNA, 1183 bp, Arabidopsis thaliana
  LTP1 (Lipid Transfer Protein 1) promoter
gatatgaccaaaatgattaacttgcattacagttgggaagtatcaagtaaacaacattttgtttttgtttgatatcgggaatctcaaaaccaaagtccacactagtttttggactatataatgataaaagtcagatatctactaatactagttgatcagtatattcgaaaacatgactttccaaatgtaagttatttactttttttttgctattataattaagatcaataaaaatgtctaagttttaaatctttatcattatatccaaacaatcataatcttattgttaatctctcatcaacacacagtttttaaaataaattaattaccctttgcatgataccgaagagaaacgaattcgttcaaataattttataacaggaaataaaatagataaccgaaataaacgatagaatgatttcttagtactaactcttaacaacagttttatttaaatgacttttgtaaaaaaaacaaagttaacttatacacgtacacgtgtcgaaaatattattgacaatggatagcatgattcttattagagtcatgtaaaagataaacacatgcaaatatatatatgaataatatgttgttaagataaactagacgattagaatatatagcacatctatagtttgtaaaataactatttctcaactagacttaagtcttcgaaatacataaataaacaaaactataaaaattcagaaaaaaacatgagagtacgttagtaaaatgtatttttttggtaaaataatcacttttcatcaggtcttttgtaaagcagttttcatgttagataaacgagattttaattttttttaaaaaaagaagtaaactaactatgttcctatctacacacctataattttgaacaattacaaaacaacaatgaaatgcaaagaagacgtagggcactgtcacactacaatacgattaataaatgtattttggtcgaattaataactttccatacgataaagttgaattaacatgtcaaacaaaagagatgagtggtcctatacatagttaggaattaggaacctctaaattaaatgagtacaaccaccaactactccttccctctataatctatcgcattcacaccacataacatatacgtacctactctatataacactcactccccaaactctcttcatcatccatcactacacacatc

SEQ ID NO: 71 - DNA, 1009 bp, Lycopersicon esculentum
  RBCS3 (Ribulose 1,5-bisphosphate carboxylase, small subunit 3) promoter
aaatggagtaatatggataatcaacgcaactatatagagaaaaaataatagcgctaccatatacgaaaaatagtaaaaaattataataatgattcagaataaattattaataactaaaaagcgtaaagaaataaattagagaataagtgatacaaaattggatgttaatggatacttcttataattgcttaaaaggaatacaagatgggaaataatgtgttattattattgatgtataaagaatttgtacaatttttgtatcaataaagttccaaaaataatctttaaaaaataaaagtacccttttatgaactttttatcaaataaatgaaatccaatattagcaaaacattgatattattactaaatatttgttaaattaaaaaatatgtcattttattttttaacagatattttttaaagtaaatgttataaattacgaaaaagggattaatgagtatcaaaacagcctaaatgggaggagacaataacagaaatttgctgtagtaaggtggcttaagtcatcatttaatttgatattataaaaattctaattagtttatagtctttcttttcctcttttgtttgtcttgtatgctaaaaaaggtatattatatctataaattatgtagcataatgaccacatctggcatcatctttacacaattcacctaaatatctcaagcgaagttttgccaaaactgaagaaaagatttgaacaacctatcaagtaacaaaaatcccaaacaatatagtcatctatattaaatcttttcaattgaagaaattgtcaaagacacatacctctatgagttttttcatcaatttttttttcttttttaaactgtatttttaaaaaaatattgaataaaacatgtcctattcattagtttgggaactttaagataaggagtgtgtaatttcagaggctattaattttgaaatgtcaagagccacataatccaatggttatggttgctcttagatgaggttattgctttaggtgaaa

SEQ ID NO: 72 - DNA, 4361 bp, Arabidopsis thaliana
  STM (Shoot Meristemless) promoter
agaatgtagcaatacaaatatatgacggtaccgttatccatcaccattatatgtatatatgtataatttgataaatattcactttgtgtttcgtcgtttgcttaataaacagctcatttccatggtattgagtcttctatatgcgagagaatcagattcccgctgggataacaaaagaacaaggtactgaaaaaaatagacaaaacttttttttaaattatataagctataaaagaaaagagtatagagagagattagccctactgtttaagagggagagagtagggtcattagggctttagagagagaagacattcggactgtccccacttgcttttctgtagaataacattatttaaatcttatttttaattaaatattacaactaaaagaagaaaccaacttttaaaataaatgcagattatatgctctgacttggactaaataaaacttgcaagtaacagtttcaagtccttttgttttagaactttttctttcgtagaagtgataaatgattgccctagacctgatagattctctaaaattctacgtattacagcataagttacctcctttatttgactattagaccatccatattggtgggcttttagcaaatgttcttaacaataattttataatttattttaatgttaagaggtttgataattttttttttttaagagtgtattttgtttattaaaatgtgttttgtttcttatataagaaccaaatcttaactattttaccaattaaacattaaatttaaattttaatatctctaagaattatattaagagccaatatagatgcttttaaaaccattggttgaataaataaatctaaccttcttaattatttctgtgtgaatattttctaaattttcattttaatttagcacaatataatccatgttctaaaaagaacaattaacataatatttacaaacctaaaaagattataaaacacaattttattttttacagcttataatgttttaaagttcaggtttattttttaaaagttcaggtttattacattaggtttgacttgtaatcatcatttatcacaacgatcaaactattattacaatcacaatagtagacaaaatttaggatatatatatatatatataattatgtataaactatgaacatttaaagtgagatttttcaaaataatatataaattcaaatagaaatagactatttggttcttaaatgagagacccccgaaaaaatctttttttttttctcatcaagctgtttacatttttagatataaaatcatattctttatagtttagaatatgaattaaatagttttatatgttattaacttatcataagatatgcgtgaggttggccaaaaactcatcaattaaccaaataagaaaagtaaaattgtattttgctttgctaaaaatgtaaatatttcattgaaaaatgaaaaaggtttaggtaatacaattaagtaaatcctacaattttggttccatggcaaaagaataaaattgtattgctttggtaaaagttgatccaactaatatattcagtagaaactgcaaaactgaagaaataagtttgtttagtagaattgctttcggttatgtaatgaatatacatccaaaatggctttttagtaatgatgtcttttcatactctttccaatccctactactttcagattatttgtcctactattatagagatatacgttcgttttcaataatatgaaaagtgatatatatttaaatagtgtgatatatatataagttttgcaagtgcatcacttcccaaaatcgcataaatcattaatcatattgtcgaaaacagtataataacttcttaaacgaaaacgcagcgcaattaaaaataacaactagagataattgacaaaacattgattaatatttacctataagttaattattgtatttaaaatttatttaaagttcataaggaaaacatatgcaaaaatatttatatctaatattttgctatgttatccttttttttttttacgttatcctaattttgtttatcctaatttgttgtggttaaaatcttattattgataaaaagagaacttttttttttgtcatcataaaaaagagaacttattacttcgattttaaaattctatgagcgtaggagacaaagaaaaaaaaaataaaaaaaaaaagaagagaaaaatcacttcttttcttctttttagtccagatccaacatattttggataactaaatgaagattttttaaaaaaatatattttagggtatatataaatcataatttgaagcaaatgaaataaaatccagtttggtaatatataaatatgatttgatgggttccttgtaatctctctctatctattagtttctcagttatcttttctttgccagaaatggcagtgaaggcagtggctgaggagagagttttttttcttctttcatggggaaagtaaaactttgccttgaagatttctctcttcaatatttttctaagacttttgatttcaacgaatcactgtccttaacctaaaagcaagaaaaattagctttatactggtctttacttttttttaacatatttatttttatatagtttacttataaacatagacatacgagtatgggaatatatagtatatccaacttctaaataatatttcgaatagtgataacaaaattagcaatacatacggctagtgaaatgttgatcgaataaacggcactgatgtaatgtacttatcaattttgataattttaattgtattgtttttctttttttcccacagtattgaactagacaattaaatttaaagtaaaattatacatttctttcgttgtgtattaaagtaacatgcataatatcattttccttcgtacaatcctccaaattgacaattgatgaattactttgtcaatcgtaaatgaatttttctcaagtctgtatactattttcagggataaacaggtacaggtgtcccatgcttattctcttgatagtaacatgtgtcctatgttgagtcaattctacgttcgaagaagtgctaacaattgttaatagcctcgtatattattctaattaaaatgcctcgatagatttggttagtggtctgaatgtgattggttattttttcaagtggcaagaggtctaccatctaatattacaatcaatcgaccaaaaaggtcgagaacatgataatggtggcaaatacaaatggttcattgttgtctaatataacaagccatcagttgtcactttttaaaaacaatacagaatacaagatactttttttttaaggtaaaatgtgtgtttaatattttcgtttatataacaaataaacagttacatgttttactctatgattatatttatgacatttttcttcttcttaacaacatttttttcccataagaacatttacaatagtattaaaactttgattgcaatcaaatgttagatcacttattataaaattactaagactgctatcttttcctattgacaaaagcgaatccaatatatgttactgaaacaaatgcgtaaattatactatatggagatctatcggttaattattgagagaatctaagaaagtttttgagtacaacagtcctaataatatcttcacataccatataatatacatatatacatatacacaaatgtactttttaaaccaacatcagcatacgtatatcccatcaggaaacttagacttttgggaattcatggtatgaaaaccaaaaccaaatgacaacattcgatttgatactcccgacccatggtaaagaaataacaaattccaatatatctttcactggactttccgaggcacattccggttttctccatttcaagaaattgtcaaaaataaattgagatccggtttattacctcaaaaaagaagaagagaaattacaacattaatttccgaaaaggcataaatgagaaatcatatttcagcagaagaacacaaaagagttaagaacccacagatcacacaacctctgtccatgtctgctttttacacttttttaaaataagtttctcctaaaaagttatttcctatttataataatttccttagatttatcttcctggtctctcttctgctgcttccctctcccccataactatcactatttagaattttcaatgtggaaaaggaagctgattgttgaagcataaatcccgggagaccacttttgcattttcaaataattaaattaaaccatagatacacacacacagttacttactcttttagggtttcccaataaatttatagtactttaatgtgtttcatgatattgatgataaatgctagctgtatttacaatgggggctcct

SEQ ID NO: 73 - DNA, 1510 bp, Arabidopsis thaliana
  RD29A (Desiccation-responsive 29a) promoter
ggttgctatggtagggactatggggttttcggattccggtggaagtgagtggggaggcagtggcggaggtaagggagttcaagattctggaactgaagatttggggttttgcttttgaatgtttgcgtttttgtatgatgcctctgtttgtgaactttgatgtattttatctttgtgtgaaaaagagattgggttaataaaatatttgcttttttggataagaaactcttttagcggcccattaataaaggttacaaatgcaaaatcatgttagcgtcagatatttaattattcgaagatgattgtgatagatttaaaattatcctagtcaaaaagaaagagtaggttgagcagaaacagtgacatctgttgtttgtaccatacaaattagtttagattattggttaacatgttaaatggctatgcatgtgacatttagaccttatcggaattaatttgtagaattattaattaagatgttgattagttcaaacaaaaattttatattaaaaaatgtaaacgaatattttgtatgttcagtgaaagtaaaacaaattaaattaacaagaaacttatagaagaaaatttttactatttaagagaaagaaaaaaatctatcatttaatctgagtcctaaaaactgttatacttaacagttaacgcatgatttgatggaggagccatagatgcaattcaatcaaactgaaatttctgcaagaatctcaaacacggagatctcaaagtttgaaagaaaatttatttcttcgactcaaaacaaacttacgaaatttaggtagaacttatatacattatattgtaattttttgtaacaaaatgtttttattattattatagaattttactggttaaattaaaaatgaatagaaaaggtgaattaagaggagagaggaggtaaacattttcttctattttttcatattttcaggataaattattgtaaaagtttacaagatttccatttgactagtgtaaatgaggaatattctctagtaagatcattatttcatctacttcttttatcttctaccagtagaggaataaacaatatttagctcctttgtaaatacaaattaattttccttcttgacatcattcaattttaattttacgtataaaataaaagatcatacctattagaacgattaaggagaaatacaattcgaatgagaaggatgtgccgtttgttataataaacagccacacgacgtaaacgtaaaatgaccacatgatgggccaatagacatggaccgactactaataatagtaagttacattttaggatggaataaatatcataccgacatcagttttgaaagaaaagggaaaaaaagaaaaaataaataaaagatatactaccgacatgagttccaaaaagcaaaaaaaaagatcaagccgacacagacacgcgtagagagcaaaatgactttgacgtcacaccacgaaaacagacgcttcatacgtgtccctttatctctctcagtctctctat

SEQ ID NO: 74 - DNA, 2244 bp, Arabidopsis thaliana
  SUC2 (Sucrose-proton Symporter) promoter
aactaggggtgcataatgatggaacaaagcacaaatcttttaacgcaaactaactacaaccttcttttggggtccccatccccgaccctaatgttttggaattaataaaactacaatcacttaccaaaaaataaaagttcaaggccactataatttctcatatgaacctacatttataaataaaatctggtttcatattaatttcacacaccaagttactttctattattaactgttataatggaccatgaaatcatttgcatatgaactgcaatgatacataatccactttgttttgtgggagacatttaccagatttcggtaaattggtattcccccttttatgtgattggtcattgatcattgttagtggccagacatttgaactcccgtttttttgtctataagaattcggaaacatatagtatcctttgaaaacggagaaacaaataacaatgtggacaaactagatataatttcaacacaagactatgggaatgattttacccactaattataatccgatcacaaggtttcaacgaactagttttccagatatcaaccaaatttactttggaattaaactaacttaaaactaattggttgttcgtaaatggtgctttttttttttgcggatgttagtaaagggttttatgtattttatattattagttatctgttttcagtgttatgttgtctcatccataaagtttatatgttttttctttgctctataacttatatatatatatgagtttacagttatatttatacatttcagatacttgatcggcattttttttggtaaaaaatatatgcatgaaaaactcaagtgtttcttttttaaggaatttttaaatggtgattatatgaatataatcatatgtatatccgtatatatatgtagccagatagttaattatttgggggatatttgaattattaatgttataatattctttcttttgactcgtctggttaaattaaagaacaaaaaaaacacatacttttactgttttaaaaggttaaattaacataatttattgattacaagtgtcaagtccatgacattgcatgtaggttcgagacttcagagataacggaagagatcgataattgtgatcgtaacatccagatatgtatgtttaattttcatttagatgtggatcagagaagataagtcaaactgtcttcataatttaagacaacctcttttaatattttcccaaaacatgttttatgtaactactttgcttatgtgattgcctgaggatactattattctctgtctttattctcttcacaccacatttaaatagtttaagagcatagaaattaattattttcaaaaaggtgattatatgcatgcaaaatagcacaccatttatgtttatattttcaaattatttaatacatttcaatatttcataagtgtgattttttttttttttgtcaatttcataagtgtgatttgtcatttgtattaaacaattgtatcgcgcagtacaaataaacagtgggagaggtgaaaatgcagttataaaactgtccaataatttactaacacatttaaatatctaaaaagagtgtttcaaaaaaaattcttttgaaataagaaaagtgatagatatttttacgctttcgtctgaaaataaaacaataatagtttattagaaaaatgttatcaccgaaaattattctagtgccactcgctcggatcgaaattcgaaagttatattctttctctttacctaatataaaaatcacaagaaaaatcaatccgaatatatctatcaacatagtatatgcccttacatattgtttctgacttttctctatccgaatttctcgcttcatggtttttttttaacatattctcatttaattttcattactattatataactaaaagatggaaataaaataaagtgtctttgagaatcgaacgtccatatcagtaagatagtttgtgtgaaggtaaaatctaaaagatttaagttccaaaaacagaaaataatatattacgctaaaaaagaagaaaataattaaatacaaaacagaaaaaaataatatacgacagacacgtgtcacgaagataccctacgctatagacacagctctgttttctcttttctatgcctcaaggctctcttaacttcactgtctcctcttcggataatcctatccttctcttcctataaatacctctccactcttcctcttcctccaccactacaaccacca

SEQ ID NO: 75 - DNA, 2365 bp, Arabidopsis thaliana
  ARSK1 (Root-specific Kinase 1) promoter
ggcgagtgatggtatatttattggttgggcttaaatatatttcagatgcaaaaccatattgaatcaataaattataaatacatagcttccctaaccacttaaaccaccagctacaaaaccaataaacccgatcaatcattatgttttcataggatttcctgaacatacattaaattatttttcattttcttggtgctcttttctgtcttattcacgttttaatggacataatcggtttcatattgtaaatctctttaacctaacgaacaatttaatgaccctagtaataggataagaaggtcgtgaaaaatgaacgagaaaaaacccaccaaaacactatataagaaagaccgaaaaagtaaaaagggtgagccataaaccaaaaaccttaccagatgttgtcaaagaacaaaaatcatcatccatgattaacctacgcttcactactaagacaaggcgattgtgtcccggttgaaaaggttgtaaaacagtttgaggatgctacaaaagtggatgttaagtatgaagcggctaaggttttggatttggtctaggagcacattggttaagcaatatcttcggtggagattgagtttttagagatagtagatactaattcatctatggagacatgcaaattcatcaaaatgcttggatgaattagaaaaactaggtggagaatacagtaaaaaaattcaaaaagtgcatattgtttggacaacattaatatgtacaaatagtttacatttaaatgtattattttactaattaagtacatataaagttgctaaactaaactaatataatttttgcataagtaaatttatcgttaaaagttttctttctagccactaaacaacaatacaaaatcgcccaagtcacccattaattaatttagaagtgaaaaacaaaatcttaattatatggacgatcttgtctaccatatttcaagggctacaggcctacagccgccgaataaatcttaccagccttaaaccagaacaacggcaaataagttcatgtggcggctggtgatgattcacaatttccccgacagttctatgataatgaaactatataattattgtacgtacatacatgcatgcgacgaacaacacttcaatttaattgttagtattaaattacatttatagtgaagtatgttgggacgattagacggatacaatgcacttatgttctccggaaaatgaatcatttgtgttcagagcatgactccaagagtcaaaaaagttattaaatttatttgaatttaaaacttaaaaatagtgtaatttttaaccacccgctgccgcaaacgttggcggaagaatacgcggtgttaaacaatttttgtgatcgttgtcaaacatttgtaaccgcaatctctactgcacaatctgttacgtttacaatttacaagttagtatagaagaacgttcgtacctgaagaccaaccgacctttagttattgaataaatgattatttagttaagagtaacaaaatcaatggttcaaatttgtttctcttccttacttcttaaattttaatcatggaagaaacaaagtcaacggacatccaattatggcctaatcatctcattctcctttcaacaaggcgaatcaaatcttctttatacgtaatatttatttgccagcctgaaatgtataccaaatcatttttaaattaattgcctaaattattagaacaaaaactattagtaaataactaattagtcttatgaaactagaaatcgagatagtggaatatagagagacaccattaaattcacaaaatcatttttaaattacctaaattattacaacaaaaactattagacagaactaagtctataatgaaacgagagatcgtatttggaatgtagagcgagagacaattttcaattcattgaatatataagcaaaattatatagcccgtagactttggtgagatgaagtctaagtacaaacaactgaatgaatttataatcaataatattgattatattgtgattagaaaaagaaaacaacttgcgttatttttcaatattattgtgaggattaatgtgaacatggaatcgtgtttctcctgaaaaaaatatcagcatagagcttagaacaatataaatatatccaccaaaaataacttcaacatttttatacaactaatacaaaaaaaaaaaagcaaactttttgtatatataaataaatttgaaaactcaaaggtcggtcagtacgaataagacacaacaactactataaattagaggactttgaagacaagtaggttaactagaacatccttaatttctaaacctacgcactctacaaaagattcatcaaaaggagtaaaagactaactttctc

SEQ ID NO: 76 - DNA, 1176 bp, Arabidopsis thaliana
  CUT1 (Cuticular Wax Condensing Enzyme1) promoter
tgtgaattatattttactcttcgatatcggttgttgacgattaaccatgcaaaaaagaaacattaattgcgaatgtaaataacaaaacatgtaactcttgtagatatacatgtatcgacatttaaacccgaatatatatgtatacctataatttctctgattttcacgctacctgccacgtacatgggtgataggtccaaactcacaagtaaaagtttacgtacagtgaattcgtctttttgggtataaacgtacatttaatttacacgtaagaaaggattaccaattctttcatttatggtaccagacagagttaaggcaaacaagagaaacatatagagttttgatatgttttcttggataaatattaaattgatgcaatatttagggatggacacaaggtaatatatgccttttaaggtatatgtgctatatgaatcgtttcgcatgggtactaaaattatttgtccttactttatataaacaaattccaacaaaatcaagtttttgctaaaactagtttatttgcgggttatttaattacctatcatattacttgtaatatcattcgtatgttaacgggtaaaccaaaccaaaccggatattgaactattaaaaatcttgtaaatttgacacaaactaatgaatatctaaattatgttactgctatgataacgaccatttttgtttttgagaaccataatataaattacaggtacgtgacaagtactaagtatttatatccacctttagtcacagtaccaatattgcgcctaccgggcaacgtgaacgtgatcatcaaatcaaagtagttaccaaacgctttgatctcgataaaactaaaagctgacacgtcttgctgtttcttaatttatttctcttacaacgacaattttgagaaatatgaaatttttatatcgaaagggaacagtccttatcatttgctcccatcacttgcttttgtctagttacaactggaaatcgaagagaagtattacaaaaacatttttctcgtcatttataaaaaaatgacaaaaaattaaatagagagcaaagcaagagcgttgggtgacgttggtctcttcattaactcctctcatctaccccttcctctgttcgcctttatatccttcaccttccctctctcatcttcattaactcatcttcaaaaatacc

SEQ ID NO: 77 - DNA, 922 bp, Lycopersion esculentum
  RSI1 (Root System Inducible 1) promoter
caatcaactaaatggacttttcttgtgcattggtcccatttttacgccctaatattcgcttacttgcttttttgtattttatttattttagttttaattttatctacctccaaattgatagaaataattacacttatagtccttttgaaaaattataattatagcattcaagtaaataaaaatacgtatttttagtcactttgtaatgtataattttgagttgaaaatgtatcaaaagtaaatttatattcttaagatatggataaagtttacatatacattatccgtttcataccctatttatagtattacattgcataagttattgtagatcttgatcgaaagtatgtgatattaatactatttttagaattatgttattctcagttatggagtgatatttaaaatcaatatagtatatcgataatcagatagtttaattcttattttctccatccaatttatataatgatattataatcaattttacgaatgagatggatattttgaaatttttagtttaaaataaattttaaattttttgtgggtctataaattatctaattaagaggtaaaatagaaagtttgaaattaattattacttactaaatatataaatatgtcattttttcttaaactgatttagaagaaaagagtgtcatatacatggacagaacgaatataatttgataattaaatttgtaaagattcatagttaatagggatcaaaattgcacgtatccattactataaggtcatatttgcttcataaaaatcatcaggatcaaaaatcagaatttatattatatttgagggactaaaaatgctaatatcacaaattaaaattagtctataaatattcacactttactcttctaattccatcaaatatttccatttatcttctcttcttcttaaatat

SEQ ID NO: 78 - DNA, 3446 bp, Arabidopsis thaliana
  AP1 (floral meristem-specific) promoter
cacggaccttggatctgaagttatgaacaataacatatttggcaaaacaaagaaaaaagaaacaacaatactaacatattttggtaaaagaacattgagaagtctcaaaaattaacttcttcttattttgtttcctaataagaccgtttgcttcatttcaagttcttaggaaataatttcatgtaacgtgtatgtagatatgtttatgtacagataaagagagatctgaaaatgatatatagagcttttgtggtgataagtgcaacaagcaggatatatatatcgaacgtggtggttagaagatagcgtcaaaatagatgctagctgctgcgtatacatcatattcatatcatatgtacttctcttttgtgatttctcatgtgattgaacatactacataaatcttgatagatttataaaaatgcaacaaattgttgtttatataagaaaaataaaacactgatatgatatttcattagttattatcaaatttgcaatataatgtttaacatccaagatttgttttacataatcgttacggttactaaagtttaatttatgatgttttaaaacaaattgagactaaatttctaaaagaaacatatacgtacatgtgtgtagctgcgtatatatatagaatggtggggctaaaagctaatgatgtgtacattaattggacatttgatgtggctggattggacccaacttgctctttgatagagacctaactaagacaattttgctcttcattcatttctcccgtatacataattgaattaactgtacataatgtttcacaacaagcgatctagctatatatttcaaaataacagagactgatattttaatctggtcttctaagctctaacgtcaaattaaaaaaaaaatccgatcttctaattaattagaagaaatcaattatagaacctctctctttaatttcatttatttaaaactgcttggaaatttaattattcactaaagactcactattctccttaatttatgataatttgtagatcatatgttcagtttttatttatttgccattcgaatgttgagttttaattaaaccaatatgttaatattcgaattaaaaaaacttacctataattcacttatttaaaaacataaaataataataattgcatcaccgtgatacaaagcaacctcacaagtcacaactctcgtgactacaaagatcactcattaaacaaaccttcctgccttctttttttctacttgggcacctcgaccgatcgaagactattcttgggatctgcttcaaaaacgactatatgttctaaatccacttcgtatgatgacgaacatttggtttactactgaagatagagattacgtccttctaattagaagtaattaattattttagtatttggaagctaatggtggagatgtaaccgtatcttagtggatcgagatattgtatataaaatatgtatgctacatcgaataataaactgaaagagagtaaaaagggatatttaatgggaagaaaagaagggtggagatgtaacaaaggcgaagataatggatattcttgggatgttgtcttcaaggccacgagcttagattcttttagttttgctcaatttgttaagtttctacttttccttttgttgcttactacttttgctcatgatctccatatacatatcatacatatatatagtatactatctttagactgatttctctatacactatcttttaacttatgtatcgtttcaaaactcaggacgtacatgtttaaatttggttatataaccacgaccatttcaagtatatatgtcataccataccagatttaatataacttctatgaagaaaatacataaagttggattaaaatgcaagtgacatctttttagcataggttcatttggcatagaagaaatatataactaaaaatgaactttaacttaaatagattttactatattacaattttttctttttacatggtctaatttatttttctaaaattagtataattgttgttttgatgaaacaataataccgtaagcaatagttgctaaaagatgtccaaatatttataaattacaaagtaaatcaaataaggaagaagacacgtggaaaacaccaaataagagaagaaatggaaaaaacagaaagaaattttttaacaagaaaaatcaattagtcctcaaacctgagatatttaaagtaatcaactaaaacaggaacacttgactaacaaagaaatttgaaacgtggtccaactttcacttaattatattgttttctctaaggcttatgcaatatatgccttaagcaaatgccgaatctgttttttttttttttgttattggatattgactgaaaataaggggttttttcacacttgaagatctcaaaagagaaaactattacaacggaaattcattgtaaaagaagtgattaagcaaattgagcaaaggtttttatgtggtttatttcattatatgattgacatcaaattgtatatatatggttgttttatttaacaatatatatggatataacgtacaaactaaatatgtttgattgacgaaaaaaaatatatgtatgtttgattaacaacatagcacatattcaactgatttttgtcctgatcatctacaacttaataagaacacacaacattgaacaaatctttgacaaaatactatttttgggtttgaaattttgaatacttacaattattcttctcgatcttcctctctttccttaaatcctgcgtacaaatccgtcgacgcaatacattacacagttgtcaattggttctcagctctaccaaaaacatctattgccaaaagaaaggtctatttgtacttcactgttacagctgagaacattaaatataataagcaaatttgataaaacaaagggttctcaccttattccaaaagaatagtgtaaaatagggtaatagagaaatgttaataaaaggaaattaaaaatagatattttggttggttcagattttgtttcgtagatctacagggaaatctccgccgtcaatgcaaagcgaaggtgacacttggggaaggaccagtggtccgtacaatgttacttacccatttctcttcacgagacgtcgataatcaaattgtttattttcatatttttaagtccgcagttttattaaaaaatcatggacccgacattagtacgagatataccaatgagaagtcgacacgcaaatcctaaagaaaccactgtggtttttgcaaacaagagaaaccagctttagcttttccctaaaaccactcttacccaaatctctccataaataaagatcccgagactcaaacacaagtctttttataaaggaaagaaagaaaaactttcctaattggttcataccaaagtctgagctcttctttatatctctcttgtagtttcttattgggggtctttgt

SEQ ID NO: 79 - DNA, 4801 bp, Arabidopsis thaliana
  AS1 (emergent leaf primordia-specific) promoter
ggaccgtgtaatgggccattgggccaagttttcttgatataaaatctgaaatactactaaattacaatttttcttaaactcgatttcataattcatgtgggactcagttctccgcgtcttatgacttaagagttaagagtaaagacaattgattgtagtttgcattattaaggttgtgattttaaaggctatattggcccaggcaaagtggttatgaaagttaaaaggtattattaaatgtcgttatggactagctaaagaaaagagatggatatagaaacggatttgccagtttgtgaggttacgtactcgttactttctattgcatttttgtgtgtcattgtgcttgtgatttctttagtatatgtttttctttttgtcaaactctttagtacatgttatgctttattttcttgtttagcattgttattgttattttgatccatgttcttttacttaatgtgtagagtgttcacgtacgactctttatgatcgctatactaatatactatgaaactcgaatgagaacatgcatgtcataaatcaataaaacataacatacgacacttaacctaaatcatacattcattgattcatactatcatgatcctcatcacattagtatcatttgtctttatttattacttagctacttcgttatcttattatatctttacctgttctgctggtcatttgccataaacaccaagtacaagcaactctttagtccaatatcagaccaaattaacaaacatttccccaatccaaaacggaaatttaattataattagcatttaaataggttcgattacaaaaaaaaatcaacaaaggaacaagtcaatttcataatggtttgtcaattgtcacacaacgaaatggctagccggatcaagcatgcatgatccaaatttcaacatttccatgataacctgaattataacgtctacataaaccatatttaaataaataggatggtcgaaagatatcattaaaagaacgattcaatattctttattgttcaattgatacacatgttattctccttaaccagttatgaacatgtcctacaagtttcttgacccaaactcataatttcatataccataatcccaagttaagttttttttttttggggatcaaaatctcaagttaagttaagttcaattatttagctgtaatgctcggaaaaaagatcggatgaatatccaattggttcaatatataccccaatccggccaatctccctatctttatagcttaattattagagaatggtcaattcacgccatcagaaccagtttcatatcttcatgaaccaaaacgcctacaaccctattattcaagaaatcactataattgtccaagtaaaaccattaattaaccgagtcgatttttctatggtcctataggcatgttgttactcaaactactgattaattaataagaagttgtagtttgaaaaagaatctagctgaaaaatactcctactctaagaatttaagttagaataaaacatattaatacaaatataaaaatttagttattaaaaaagcgctactaccaagacgtcctaaagaaaaactagctttgtcttctaaaagaaaacctagcttaactacccaaaaaaatctagttttacaaacactaaagacaaattttatttttcaacaaatttaccaattaaagaaaattccatgtaggaatgtatccaaattgaaaatatccctacatattttgtaggaaaaaaggtttttataaatattaaaaaaacgagaaaaagaaaagagaaaagagaaaaaaaaaagccggagagaatggagcacatgaggtaaaaggcaagagatggcagagagaagatcagagaagggatctgcctcaatttgacaactcatatgtcatgtcatttccctcactactattattttcctatttcaaaaacacctttctctgataccatcaccttttaccttctctttttttttactgtctttgctctgtttcacattcccttctatatatacagtatagtatattttatccttcttttattgttttgcttactaaaagtttttttcctccggaatcaaaattctaaaatgtatatcatgttaggtcgcgagggccatgcaatattatgaactatgcatgatgattaatgtctgtggatccatcacaaatattattgaaggttgatcagagactatggaccaaaatggtccgaatcgcctgataataaaaaactattcatttttattttttattttttttattaaacatgtgattaatgatagatcttacgattcgcaactgggaaacatgcactaactcaaacttaaaacacacaatactaaaagttctattaaattttgaatgtaaagagaaatatattaggcaatcaaacggtcaagtaaatcatacacatcgataatttatttttttatccttcaaagcaggcccatccaaggcccaccactattctcatatcaacatacttttcttgttttggttaaatcaacctaccatgttggctgttctctccgctcctctgtgtaagatcacaccaacaccactgcataatttcttgtattattttgagacttgagagtaaactgattgacaaaaaaaaaaaaaaaaaaaaaaaattgagagtaaactagtttcttgaatattgattttttcagcttaatttgttggggaaagatattactactattgctgtaaaaaaaaaaaaaaaaaaaaaagatattattactatatttgtagtgattttattttgaaaattctcttcacttttttgtagttaacattctaattttgtgaaaagaacttttaatgtcaggtcatgtctcttaaaaagtttgcatgatgaaatgatttacaaattacaatagaaaatggaaaccattgcaaactaaatttttatcaaaaaaaatcgaaaataaaatgtattgacttagtaatgctgtgtctgctacgattaactattacacataatgcaacactgaattatccaaatacattattagaataatagtattacagtatcactattacaacaacaatgtcaacaataatcttattataataatatataaatagaccttagtgacatcatattatatagaaaacatgtggttgcctaatttgtataagctagatacttgggggtgatgagtgactagttgatgcaatgataaaagagtgaaagttttgtctgcctgattatagacgtcggagaaatactaaaatacgctatgaagattttggcgcatggtagcagaaaaaaaaaacggagggtgtgagtgagtagtggtagtcggatgtgatggaacaaagaaaagtatttttggtagggttatgggagagagaaggggaccattattacacacttacatgctttccccaaaagataccattcccattttctgacacgtgtccccctcatccccaattactcatacgtcaaatccaatttttagcctaaaagttttttttatttgtttagccaaatctattttactaattaaagttttcaaatggcaaatagaaagatcttctaaggttttataaaattacttgattatttctagttttgctcattttttaaataaaatttctcttttttttcttgcaacattattgattttttttttgatagggagtaacattagtgatgttctatctcttctcattgcaaaaactttattttctcatctctatttgatcatcattgcgaaatcttccattttcaacaaatacttttccatgttaatatgctgtttcaaaatataagtgtttggaaaataaatcaacaagtttaaatgttaactatttttatgctattataattatttttcttatgggtaagtggaaattaatgttactcaaattggacataaaattctattgtttgagtgaaggagtttataaatggagcattattttcttgaatggttagtttttcttctatcattttgacaagtaaatgacttttcagccactaaagtacaacactttttcatttaaatttaaagcatcccctacattagattgtcattttatttctcataatgttatagaaaaatgaattttgagatcccaatgtagtaaatatatataaaaaaaggtttaatattgtcaatgacaaacaacgaacttatggaatttcaacttttcacctccacgcgcctctgtcagagttttttttttccccacttgtgatgtaaaaaggggaaaacgtctgtgtctcagtcggtaaactttttctctctttttttttttaaagattttattttaattatgccgtctctgtggtctaatcgtgtacgtcgtctggttttaaaagcctctctcactttggtcttttcgttttctctcttccattttctccaactatataaaaaaaaaaaagtgagagagagagcaaatctgtgtgatggaagttgctcttgagtttgggattatttatcttttcaatatcatttggtaagcatttttattttgttttatagtaataattttaactctcttatcttcttaataagtctttgcttaatagtgttttggggtcagcattaatttcccctgtttggtttccagaatataggttgtatagtgtgataataacaaattattccaagttttgcttcaaacattgtcaaagtttttgtcattttcatttcttgaaacggaaatttttcagactttgtaatttctaattcgaaaattcgacagatcttgtagatttgtttcgatcttttagagttttgaattggagagatttatgaaacgggttgatttt